(19) United States
(12) Patent Application Publication
(10) Pub. No.: US 2026/0369382 A1
(43) Pub. Date: Nov. 19, 2026

(54) Denoising Diffusion Models for Digital Oral Care

(71) Applicant: Solventum Intellectual Properties Company, Maplewood, MN (US)

(72) Inventors: Jonathan D. Gandrud, Woodbury, MN (US); Lois F. Duerst, Hudson, WI (US); Michael B. Starr, Toomevara (IE); Mariah Sonja Pereira Penha, Dublin (IE); Donna J. Stenberg, Stillwater, MN (US)

(21) Appl. No.: 19/139,600

(22) PCT Filed: Dec. 14, 2023
(86) PCT No.: PCT/IB2023/062713

§ 371 Date: Jun. 16, 2025

Related U.S. Application Data
(60) Provisional application No. 63/432,627, filed on Dec. 14, 2022.
(60) Provisional application No. 63/522,774, filed on Jun. 23, 2023.

(51) Int. Cl. G16H 20/40; G06T 5/70; G06T 7/00; G06T 17/00; G16H 30/40

(52) U.S. Cl. CPC G16H 20/40; G06T 5/70; G06T 7/0012; G06T 17/00; G16H 30/40; G06T 2207/20081; G06T 2207/20084; G06T 2207/30036

(57) ABSTRACT

Systems and techniques are disclosed for automatically generating a data structure for oral care treatment using trained machine learning models. The method involves receiving one or more attributes that describe the intended output from the trained machine learning model. One or more noisy representations of the intended output are generated by one or more computer processors. These noisy representations are then denoised using a trained machine learning model, also executed by the computer processors. The denoised representations of the intended output are generated, providing more accurate and reliable data. Based on the generated denoised representations, one or more aspects of digital oral care treatments are automatically defined by the computer processors. These systems and techniques enable the creation of a comprehensive data structure for oral care treatment, enhancing treatment planning and decision-making processes in the field of oral healthcare.

### RELATED DOCUMENTS

The entire disclosure of PCT Application No. PCT/IB2022/057373 is incorporated herein by reference. The entire disclosures of each of PCT Applications with Publication Nos. WO2022123402A1, WO2021245480A1, and WO2020026117A1 are incorporated herein by reference. The entire disclosure of each of the following Provisional U.S. Patent Applications is incorporated herein by reference: 63/432,627; 63/366,492; 63/366,495; 63/352,850; 63/366,490; 63/366,494; 63/370,160; 63/366,507; 63/352,877; 63/366,514; 63/366,498; 63/366,514; 63/264,914; and 63/432,627.

### TECHNICAL FIELD

This disclosure relates to configurations and training of denoising diffusion models (e.g., such as neural networks which have been trained for that purpose) to improve the accuracy and data precision of 3D oral care representations to be used dental or orthodontic treatments.

### SUMMARY

The present disclosure describes systems and techniques for training and using one or more denoising diffusion probabilistic models (e.g., neural networks such as U-Nets or autoencoders) to generate 3D oral care representations. Denoising diffusion-based techniques are described for the placement of oral care articles in relation to one or more 3D representations of teeth. Oral care articles which are to be placed may include: a 3D representation of a tooth (e.g., for the generation of an orthodontic setup), a dental restoration appliance component, oral care hardware (e.g., a lingual bracket, a labial bracket, an orthodontic attachment, a bite ramp, etc.), and the like. Furthermore, denoising diffusion-based techniques are described for the generation of the geometry and/or structure of oral care articles based, at least in part, on one or more 3D representations of teeth. Oral care articles which may be generated include: a dental restoration tooth design, a crown, a veneer, an archform, a clear tray aligner (CTA) trimline, and an appliance component (e.g., such as generated components for use in creating a dental restoration appliance) and the like. Of the neural networks which may be trained to perform denoising diffusion, U-Nets are an example of models that may enable improvements to data precision. Denoising diffusion models may be trained to automatically generate (or modify) 3D oral care representations, such as 3D point clouds (or other 3D representations described herein). In some instances, a denoising diffusion model may be trained to generate 3D polylines (e.g., for archforms and CTA trimlines), or sets of control points (e.g., control points through which a spline can be fitted for an archform). A denoising diffusion model may be trained to predict an archform (e.g., taking dental arch and tooth data as input). An archform may take the form of a surface, a 3D mesh, a 3D polyline or a set of control points (e.g., used to define a spline). Such an archform may, in some instances, be given as an input to a setups prediction machine learning model, such as a setups prediction neural network. A denoising diffusion model may be trained to label aspects of a 3D representation (or to generate one or more object masks which identify one or more objects within the input 3D representation), for use in segmentation or mesh cleanup of 3D oral care representations.

Techniques of this disclosure include methods of a generating a data structure for oral care treatment. Such methods may receive as input one or more attributes describing an intended output from a trained machine learning model. Such methods may generate one or more noisy representations of an intended output, and subsequently denoise the one or more noisy representations. The output of the method may include one or more generated denoised representations, which may be used to define one or more aspects of one or more digital oral care treatments. A training dataset may be generated or refined by successively modifying one or more 3D oral care representations. An untrained (or partially trained) machine learning model may be trained, at least in part, using the refined training dataset. The resulting trained machine learning model may be deployed for clinical treatment of patients. Through the course of generating the training dataset, one or more representations of the training dataset may be modified, such as through the addition of noise. In some implementations, one or more aspects of one or more representations of the training dataset may be encoded into a latent form before generating the one or more noisy representations. The one or more attributes may include at least one of a real value, a categorical value, or a natural language text value. The one or more attributes may include at least one of an oral care metric or an oral care parameter. The trained machine learning model may include at least one neural network. At least one neural network may include at least one encoder-decoder structure. An encoder-decoder structure may comprise at least one encoder or at least one decoder. Non-limiting examples of an encoder-decoder structure include a U-Net, a transformer, a pyramid encoder-decoder or an autoencoder, among others. The denoising diffusion techniques may receive one or more 3D representations of the patient's dentition (e.g., which may include at least one tooth). The one or more 3D representations of the patient's dentition may be encoded into a latent form. The one more denoised representations (aka generated 3D oral care representations) may include at least one of: one or more 3D oral care representations as defined herein, at least one label for a mesh element, at least one transform to place at least one tooth of a patient's dentition into a setup pose, an appliance component, a trimline, an archform, a coordinate system, or a tooth restoration design (among others). A label on a mesh element may be used to segment one or more 3D oral care representations (e.g., to segment at least one representation of a patient's dentition). A label may be used for the cleanup of a 3D oral care representation (e.g., the label may be used to modify one or more 3D oral care representations-such as a representation of a patient's dentition). A transform predicted using the techniques described herein may place a tooth in a setup pose. In some implementations, a setup may correspond to a final setup. In some implementations, a setup may correspond to an intermediate stage. In some instances, an appliance component (e.g., that is placed or generated using a denoising diffusion model) may be used to generate one or more dental restoration appliances.

The denoising diffusion techniques described herein may, in some instances, be practiced in combination. For example, denoising diffusion mesh cleanup may be performed on a dental arch from an intraoral scanner, after which denoising diffusion segmentation may be performed on that cleaned-up 3D representation of the patient's dentition. In some instances, one or more of the segmented teeth may be identified for dental restorative treatment and subsequently provided to a denoising diffusion model for geometry generation (e.g., Geometry Generation Diffusion Model GGDM), to generate a dental restoration tooth design. In some implementations, the segmented teeth of the dental arch may be provided to a Diffusion Setups model for setups prediction. Other combinations of the techniques described herein should also be considered within the scope of this disclosure.

Techniques of this disclosure may generate data structures for oral care treatment using denoising diffusion probabilistic models. The methods may receive oral care arguments (or oral care attributes) describing the intended output from a trained denoising diffusion model. The methods may execute a forward pass to generate a series of increasingly noisy versions of each training example. In deployment, a denoising ML model (e.g., a U-Net) may be trained to iteratively denoise an initially noisy data structure. After many iterations of denoising, the data structure may evolve into a 3D oral care representation which is suitable for use in treating the patient. Stated another way, one or more denoised representations are generated, and based on these representations, one or more aspects of digital oral care treatments are automatically defined (e.g., a restoration design for a tooth is generated, or an orthodontic setup is generated, among examples).

The method may access a training dataset of 3D oral care representations and generate a refined training dataset by modifying representations of the training dataset (e.g., iteratively adding noise). An untrained machine learning model may be trained using the refined training dataset, and the trained machine learning model is outputted based on the training.

The modifying of the representations of the training dataset involves adding noise to aspects of the representations. Furthermore, the method encodes aspects of the representations into a latent form before generating the noisy representations.

The attributes describing the intended output can include real values, categorical values, or natural language text values. Additionally, the attributes can include oral care metrics or oral care parameters.

The trained machine learning model can include neural networks such as U-Nets, transformers, or autoencoder (or the like). The method can receive 3D representations of a patient's dentition, and the denoised representations can include 3D oral care representations. These representations can be encoded into a latent form (e.g., when Stable Diffusion is used).

The methods may also be trained to label mesh elements and apply those mesh element labels to 3D representations of oral care data, to segment those 3D representations based on the labels. The 3D oral care representations can include representations of a patient's dentition, among others described herein.

Furthermore, the denoised representations can include transforms (e.g., 4×4 transformation matrices, among others), which may be used to place a patient's tooth into setup poses (e.g., for final setups or intermediate stages). In some implementations, the transforms may place other 3D representations of oral care data into poses which are suitable for oral care treatment. Other 3D representations of oral care data include fixture model components, tooth restoration designs, appliance components, fixture model components, or archforms (or the like). Additionally, the denoised representations can include coordinate systems, or other 3D oral care representations described herein.

In summary, the disclosed methods utilize denoising diffusion probabilistic models to generate data structures for use in oral care treatment, allowing for automatic definition of various aspects of digital oral care treatments based on trained models and denoised representations.

Stated another way, methods of this disclosure may generate data structures for use in digital oral care treatment. The methods may define one or more attributes (or oral care arguments) describing an intended output from a trained machine learning model. The methods may generate training datasets by generating one or more noisy representations of the intended output (e.g., a series of increasingly noisy representations), and/or training machine learning models using the one or more noisy representations of the intended output. The methods may use a trained model (e.g., a hierarchical neural network feature extraction model-such as a U-Net) to iteratively denoise a representation (e.g., a representation which initially has a random, Gaussian or other default value or distribution). The methods may generate (or modify) one or more denoised representations of the intended output, after one or more iterations of denoising. The methods may generate one or more oral care appliances using the denoised representation, or otherwise use the denoised representation for one or more aspects of one or more digital oral care treatments. A refined dataset may be generated by successively modifying one or more representations of the training dataset. An initially untrained machine learning model may be trained using the refined training dataset, and outputted for use in oral care appliance generation. The refined training dataset may be generated by adding noise to aspects of the one or more representations of the training dataset. In some implementations, one or more aspects of one or more representations of the training dataset may be encoded into a latent form before generating the one or more noisy representations. Stated another way, each example of the training dataset may first undergo latent encoding (e.g., using an encoder) before the refined dataset (of iteratively noisier examples) is generated. The one or more oral care attributes (or oral care arguments) may describe the shape, structure, layout, or other properties of an intended output. Oral care arguments may include oral care metrics, and/or oral care parameters. Other oral care attributes may include doctor preferences. The one or more oral care attributes (or oral care arguments) may include at least one of a real value, a categorical value, or a natural language text value. The trained machine learning model may include at least one neural network, such as a hierarchical neural network feature extraction model (e.g., a U-Net), an autoencoder, or a transformer (e.g., 3D SWIN transformer). One or more 3D representations of the patient's dentition (e.g., one or more teeth of the patient) may be provided to the methods. The methods may denoise a 3D oral care representation (e.g., a 3D oral care representation which is to undergo modification). In some implementations, the 3D oral care representation (e.g., 3D representations of the patient's dentition) may be encoded into a latent form. In some instances, the one or more denoised representations includes at least one or more labels for a mesh element (e.g., for use in mesh segmentation or mesh cleanup). The methods may segment a 3D representation (e.g., a mesh, point cloud, voxelized representation, etc.) using the one or more labels. One or more 3D representations of oral care data may be segmented or undergo mesh cleanup (e.g., 3D representations of a patient's dentition). Mesh cleanup may involve using the one or more labels to modify the one or more 3D oral care representations (e.g., to remove mesh elements which have a particular label value, or to modify mesh elements which have a particular label value). The methods may, in some implementations, denoise one or more representations of transforms (e.g., transforms which may place teeth into setups poses-such as final setups or intermediate stages, place appliance components for appliance generation, or place fixture model components for fixture model generation). The methods may, in some implementations, denoise one or more 3D representations of a tooth restoration design, to modify the shape and/or structure of a pre-restoration tooth and make that tooth suitable for use in digital oral care treatment (e.g., to use in the generation of a dental restoration appliance). The methods may, in some implementations, denoise one or more 3D representations of appliance components, and make the one or more appliance components suitable for use in digital oral care treatment (e.g., to use in the generation of a dental restoration appliance). The methods may, in some implementations, denoise one or more 3D representations of fixture model components, and make the one or more fixture model components suitable for use in digital oral care treatment (e.g., to use in the generation of a digital fixture model). The digital fixture model may be 3D printed, resulting in a physical fixture model. Orthodontic aligner trays may be thermoformed using the physical fixture model and used in orthodontic treatment of the patient. The methods may, in some implementations, denoise one or more 3D representations of clear tray aligner trimlines, and make the one or more clear tray aligner trimlines suitable for use in digital oral care treatment (e.g., to use in trimming an aligner tray off of a physical fixture model). The methods may, in some implementations, denoise one or more 3D representations of an archform, and make the one or more archforms suitable for use in digital oral care treatment (e.g., to use in the generation of an oral care appliance). The methods may, in some implementations, denoise one or more 3D representations of a coordinate system, and make the one or more coordinate systems suitable for use in digital oral care treatment (e.g., to use in the generation of an oral care appliance).

### BRIEF DESCRIPTION OF DRAWINGS

FIG. 1 shows a method of using a denoising diffusion probabilistic model to generate (or modify) a 3D oral care representation.

FIG. 2 shows a method of training a denoising diffusion probabilistic model to generate (or modify) a 3D oral care representation.

FIG. 3 shows a method of generating orthodontic setups transforms using denoising diffusion probabilistic models.

FIG. 4 shows a method of using a hierarchical neural network feature extraction module to perform mesh element labeling (e.g., for segmentation or mesh cleanup) of a 3D representation, according to the denoising diffusion probabilistic models of this disclosure.

FIG. 5 shows a U-Net structure, which may be used to extract hierarchical features from a 3D representation.

FIG. 6 shows a pyramid encoder-decoder structure, which may be used to extract hierarchical features from a 3D representation.

### DETAILED DESCRIPTION

Diffusion models may be applied to 2D image generation, or the generation of 3D representations, among others. In some instances, such implementations may take input from natural language text (e.g., natural language text, real values, categorical values, reference images or reference 3D representations which describe an intended outcome). The techniques described herein expand diffusion models into the digital oral care space. The techniques described herein use a diffusion model to generate 3D oral care representations (e.g., as 3D point clouds, 3D meshes, 3D voxelized representations, 3D surfaces and the like) based on inputted arguments (e.g., which may include natural language text, integer arguments, real-valued arguments, categorical arguments, and the like). Such arguments may contain one or more attributes describing an intended output from a trained machine learning model. Techniques described herein may, in some instances, take as input representations of the patient's dentition (e.g., 3D point clouds, 2D views or 2D digital photographs), which are to be used as guides for the generation of artifacts which are to be used in digital oral care. Techniques described herein may, in some instances, take as input representations of appliances, appliance components, trimlines, archforms or other 3D oral care representations (e.g., in the form of 3D point clouds, 2D views or 2D digital photographs) which are to be modified or used as templates or references for artifacts which are to be generated for use in digital oral care. In some instances, these inputs (e.g., teeth, gums, appliances, appliance components, archforms, trimlines for the refinement of manufactured trays-such as printed or thermoformed trays, segmentation masks on mesh elements, or other 3D oral care representations) may be encoded into a latent (e.g., information-rich reduced dimensionality) form by autoencoders or by other encoder-decoder neural networks. In some instances, a trimline may be generated which is used to cut a thermoformed tray off of a fixture model (e.g., a fixture model used in the manufacture of either an indirect bonding tray or an orthodontic aligner). In some instances, techniques of this disclosure may realize improved data precision through the use of such latent representations. These techniques are valuable to 3M's digital oral care platforms, particularly to custom smile design in the form tooth restoration design generation, appliance component generation, aligner trimline generation, setups prediction and the like.

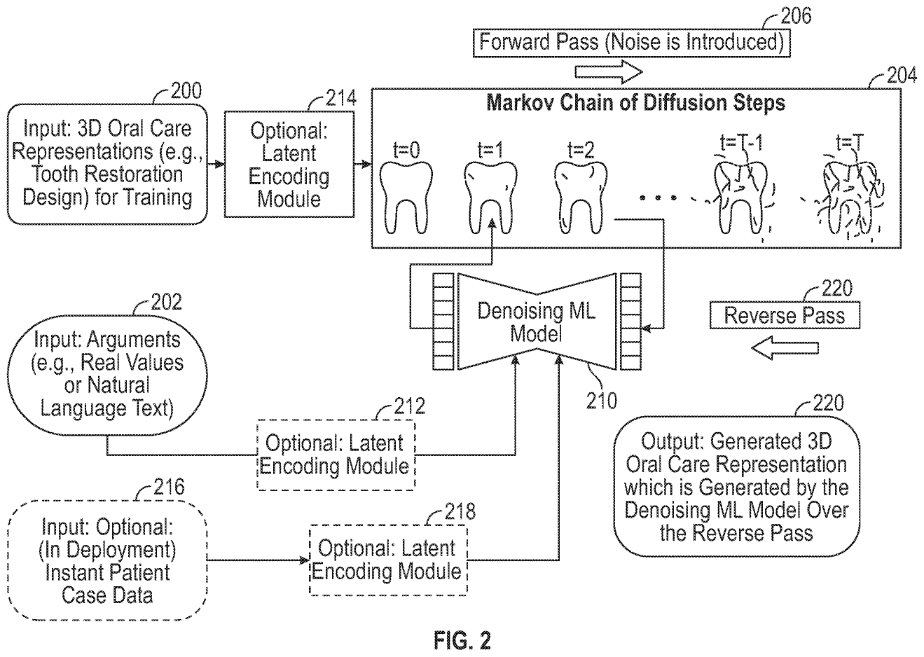

A denoising diffusion model (e.g., shown in FIG. 2) may involve a forward pass 206 over the input data 200 (e.g., a 3D point cloud of a tooth, a transform, or another 3D oral care representation described herein) which may generate a Markov chain of steps 204 which may introduce successively more noise (e.g., Gaussian noise) to the input data 200. The forward pass 206 may generate training data. The Markov chain may generate a set of successively noisier training data examples. Input oral care arguments (or attributes) 202 may influence the functioning of the denoising diffusion model, causing the denoising diffusion model to generate output to the specification of the clinician (e.g., enabling the customization of the output that is generated by the denoising diffusion model). Oral care arguments may include oral care parameters, oral care metrics, among other examples. In some implementations, such as with stable diffusion, an optional latent encoding module 214 may encode 3D oral care representations 200 into latent form. A latent encoding module may be trained to encode data into a reduced dimensionality latent form. Examples of data which may be encoded include a transform (e.g., a tooth transform, etc.), a point cloud or mesh describing a tooth, a set of mesh element labels. Such data may be encoded into a latent vector or latent capsule. Likewise, an optional latent encoding module 212 may, in some implementations, encode one or more of oral care arguments 202 into latent form.

The Markov chain may generate a succession of increasingly noisy versions of the input data 200, which may then be used to train, at least in part, a denoising ML module 210 (e.g., which may function as a part of the reverse pass 208). The denoising ML model which is trained for use in reverse pass may comprise one or more neural networks 210 (e.g., U-Net, VAE, 3D SWIN transformer, pyramid encoder-decoder, or the like) and may be trained to de-noise a highly noisy version of the data (e.g., starting with a completely randomized version of the input data structure and de-nosing that data structure until the data structure takes on aspects of a suitable generated output). The reverse pass 208 may be used during model deployment (e.g., after deployment of the trained denoising ML model 210). For example, the reverse pass 208 may start with a point cloud (or other raw data structure—such as a transforms or mesh element labels) that has a Gaussian distribution, and over successive applications of the reverse pass denoising ML model 210 gradually shape that point cloud (or other data structure) into a suitable example of the target 3D oral care representation (e.g., a tooth design which is suitable for use in creating a dental restoration appliance). In some implementations, a loss function (e.g., cross-entropy or MSE, among others) may be computed to quantify the differences between a generated 3D oral care representation and a corresponding ground truth (or reference) 3D oral care representation. In some implementations, the loss function may be used to train, at least in part, the denoising ML model 210. In some implementations, such as when an orthodontic setup or a tooth restoration design is generated, oral care metrics may be computed on the generated 3D oral care representation 220. The oral care metrics may be used, at least in part, to assess the quality of fitness for use of the generated 3D oral care representation (e.g., to measure whether the generated 3D oral care representation meets the specification of the oral care arguments 202 and is ready for use in generating an oral care appliance).

Stated another way, in some instances, the forward pass 206 of the denoising diffusion model may generate a training dataset of increasingly noisy examples of the input data. Noise may be introduced to disfigure the input data (e.g., an image, a 3D point cloud, a transform, or latent representations of one or more of these inputs, etc.) and those noisy examples may be used, at least in part, to train a denoising diffusion machine learning model 210 to reverse of this noise-introducing process (e.g., in model deployment). The reverse pass 208 may be trained to reconstruct the pristine input data by removing the noise from a noisy example of that input data. After the denoising diffusion model (e.g., a U-Net) is trained, the model is capable to generate new 3D oral care representations by passing a noisy data example (e.g., a randomly generated noisy example) through the denoising diffusion process (aka the reverse process).

Techniques of this disclosure may require a training dataset of hundreds or thousands of cohort patient cases, to ensure that the neural network is able to encode the distribution of patient cases which are likely to be encountered in clinical treatment. A cohort patient case may include a set of tooth crown meshes, a set of tooth root meshes, or a data file containing attributes of the case (e.g., a JSON file). A typical example of a cohort patient case may contain up to 32 crown meshes (e.g., which may each contain tens of thousands of vertices or tens of thousands of faces), up to 32 root meshes (e.g., which may each contain tens of thousands of vertices or tens of thousands of faces), multiple gingiva mesh (e.g., which may each contain tens of thousands of vertices or tens of thousands of faces) or one or more JSON files which may each contain tens of thousands of values (e.g., objects, arrays, strings, real values, Boolean values or Null values).

In some implementations, the denoising ML model 210 of the reverse pass 208 may modify an existing 3D oral care representation (e.g., modify a pre-restoration tooth design). The existing 3D oral care representation (e.g., an example of optional instant patient case data 216) can be provided to the input of the reverse pass, and then undergo a succession of denoising steps by the denoising ML model 210, until modifications are complete (e.g., as measured by oral care metrics, loss functions, or a threshold number of iterations has expired, etc.). Optional instant patient case data 216 may include data pertaining to the dentition of a patient. The instant patient case data may, in some implementations, be introduced to customize the functioning of the denoising diffusion ML model to the anatomy of the patient. In some implementations, the instant patient case data may be encoded 218 into a latent or embedded form. In some implementations, the instant patient case data may be provided to the denoising diffusion ML model 210. In other implementations, the instant data 216 may comprise an appliance component or a fixture model component which requires modification.

In some implementations, which data structure that undergoes iterative denoising by the denoising ML model 210 may be initialized, at least in part, according to a stochastic process (e.g., using random noise or random configurations), or by aspects of the instant patient case data 216, or by a combination of the two. The instant patient case data 216 may include 3D oral care representations described herein, including tooth transforms (e.g., maloccluded transforms for one or more teeth during setups prediction), tooth meshes with transforms already applied, tooth meshes without transforms applied, one or more 3D representations of pre-restoration tooth designs, pre-segmentation dental arch mesh(es), pre-cleanup dental arch mesh(es), one or more mesh element labels (e.g., for segmentation or mesh cleanup), one or more segmented teeth for use in coordinate system prediction, one or more coordinate systems (e.g., each of which may be described by a transform), one or more 3D representations of appliance components (e.g., parting surfaces, etc.), one or more fixture model components (e.g., digital pontic teeth or interproximal webbing, etc.), or the like.

The denoising diffusion ML model 210 may be trained to generate one or more generated 3D oral care representations 220 (e.g., as defined herein) for the pat′ent's treatment. A generated 3D oral care representation 220 (aka a denoised representation) may be generated over one or more iterations of denoising by the denoising diffusion ML model 210. Generated 3D oral care representations 220 may include setups transforms for one or more teeth, transforms for the placement of one or more appliance components (e.g., for the generation of a dental restoration appliance), one or more 3D representations of post-restoration tooth designs, one or more generated (or modified) appliance components (e.g., a parting surface or gingival ribbon for use in generating a dental restoration appliance), one or more generated (or modified) fixture model components (e.g., one or more trimlines, etc.), post-segmentation dental arch mesh(es), post-cleanup dental arch mesh(es), one or more mesh element labels for use in segmentation or mesh cleanup, one or more object masks (e.g., masks to be applied to mesh elements) for use in segmentation or mesh cleanup, one or more coordinate axes for one or more predicted coordinate systems, one or more archforms, or other of the 3D oral care representations described herein.

The machine learning techniques described herein may receive a variety of input data, as described herein, including tooth meshes for one or both dental arches of the patient. The tooth data, appliance components, fixture model components, to name a few, may be provided in the form of 3D representations, such as meshes, point clouds or voxelized geometries. These data may be preprocessed, for example, by arranging the constituent mesh elements into lists and computing an optional mesh element feature vector for each mesh element. Such vectors may impart valuable information about the shape and/or structure of an oral care mesh to the machine learning models described herein. Additional inputs may be received as the input to the machine learning models described herein, such as one or more oral care metrics. Oral care metrics may be used for measuring one or more physical aspects of an oral care mesh (e.g., physical relationships within a tooth or between different teeth). In some instances, an oral care metric may be computed for either or both of a malocclusion oral care mesh example and/or a ground truth oral care mesh example which is then used in the training of the machine learning models described herein. The metric value may be received as the input to the machine learning models described herein, as a way of training that model or those models to encode a distribution of such a metric over the several examples of the training dataset. During training, the network may then receive metric value(s) as input, to assist in training the network to link that inputted metric value to the physical aspects of the ground truth oral care mesh which is used in loss calculation. Such a loss calculation may quantify the difference between a prediction and a ground truth example (e.g., between a predicted oral care mesh and a ground truth oral care mesh). By providing the network with metric value(s), the neural network techniques of this disclosure may, through the course of loss calculation and subsequent backpropagation, learn train the neural network to encode a distribution of a given metric. In deployment, one or more oral care arguments may be defined to specify one or more aspects of an intended 3D oral care representation (e.g., a 3D mesh, a polyline, a 3D point cloud or a voxelized geometry), which is to be generated using the machine learning models described herein (e.g., denoising diffusion models) that have been trained for that purpose. In some implementations, oral care arguments may be defined to specify one or more aspects of a customized vector, matrix or any other numerical representation (e.g., to describe 3D oral care representations such as control points for a spline, an archform, a transform to place a tooth or appliance component relative to another 3D oral care representation, or a coordinate system), which is to be generated using the machine learning models described herein (e.g., denoising diffusion models) that have been trained for that purpose. A customized vector, matrix or other numerical representation may describe a 3D oral care representation which conforms to the intended outcome of the treatment of the patient. Oral care arguments may include oral care metrics or oral care parameters, among others. Oral care arguments may specify one or more aspects of an oral care procedure-such as orthodontic setups prediction or restoration design generation, among others. In some implementations, one or more oral care parameters may be defined which correspond to respective oral care metrics. Oral care arguments can be provided as the input to the machine learning models described herein and be taken as an instruction to that module to generate an oral care mesh with the specified customization, to place an oral care mesh for the generation of an orthodontic setup (or appliance), to segment an oral care mesh, or to clean up an oral care mesh, to name a few examples. This interplay between oral care metrics and oral care parameters may also apply to the training and deployment of other predictive models in oral care as well.

The predictive models of the present disclosure may, in some implementations, may produce more accurate results by the incorporation of one or more of the following inputs: archform information V, interproximal reduction (IPR) information U, tooth dimension information P, tooth gap information Q, latent capsule representations of oral care meshes T, latent vector representations of oral care meshes A, procedure parameters K (which may describe a clinician's intended treatment of the patient), doctor preferences L (which may describe the typical procedure parameters chosen by a doctor), flags regarding tooth status M (such as for fixed or pinned teeth), tooth position information N, tooth orientation information O, tooth name/dental notation R, oral care metrics S (comprising of at least one of oral care metrics and restoration design metrics).

Systems of this disclosure may, in some instances, be deployed at a clinical context (such as a dental or orthodontic office) for use by clinicians (e.g., doctors, dentists, orthodontists, nurses, hygienists, oral care technicians). Such systems which are deployed at a clinical context may enable clinicians to process oral care data (such as dental scans) in the clinic environment, or in some instances, in a “chairside” context (where the patient is present in the clinical environment). A non-limiting list of examples of techniques may include: segmentation (e.g., such as diffusion segmentation), mesh cleanup, coordinate system prediction, CTA trimline generation, restoration design generation (e.g., such as using diffusion models for 3D point cloud generation), appliance component generation or placement or assembly (e.g., such as using diffusion models for 3D point cloud generation), generation of other oral care meshes, the validation of oral care meshes, setups prediction (e.g., such as diffusion setups), removal of hardware from tooth meshes, hardware placement on teeth, imputation of missing values, clustering on oral care data, oral care mesh classification, setups comparison, metrics calculation, or metrics visualization. The execution of these techniques may, in some instances, enable patient data to be processed, analyzed and used in appliance creation by the clinician before the patient leaves the clinical environment (which may facilitate treatment planning because feedback may be received from the patient during the treatment planning process).

Some existing techniques may use diffusion models for dental crown restoration. Such techniques may convert a 3D point cloud of a tooth to a 2D depth map, use a diffusion model to process the depth map, and then attempt to convert the resulting modified depth map back into a 3D point cloud of the tooth. Error may be introduced by attempting to convert between the 3D representations and 2D representations in this fashion. Stated another way, when data are converted between representations of different dimensionality (e.g., from 2D to 3D, or from 3D to 2D), noise may be introduced. The denoising diffusion probabilistic models of this disclosure can be trained to directly generate a 3D tooth restoration design (or other 3D representations described herein, such as fixture model components or appliance components) without the intervening step of depth map generation. For example, the denoising ML model 210 of this disclosure may be directly trained on a series of increasingly noisy point clouds 204 of tooth restoration designs (e.g., either pre-restoration or post-restoration tooth designs). The fully trained denoising ML model 210 may then be used to denoise a noisy (or randomly initialized) 3D point cloud (or other 3D representation) directly to generate a tooth restoration design, without the need to first convert the 3D tooth data into a 2D depth map or any other 2D representation, and then back into a 3D representation. Not only do the techniques of this disclosure generate more accurate tooth shapes and/or structures (e.g., less-noisy tooth surfaces), but the techniques of this disclosure also enable reduced resource usage. The techniques of this disclosure avoid the computational cost (e.g., compute cycles) and storage (e.g., computer memory) requirements of converting between representations of different dimensionalities (e.g., as pertain to the depth map approach). Stated another way, the techniques of this disclosure have a lower resource footprint than techniques that convert between representations of different dimensionalities as pre-processing steps.

Systems of this disclosure may automate operations in digital orthodontics (e.g., setups prediction, hardware placement, setups comparison), in digital dentistry (e.g., restoration design generation) or in combinations thereof. Some techniques may apply to either or both of digital orthodontics and digital dentistry. A non-limiting list of examples is as follows: segmentation, mesh cleanup, coordinate system prediction, oral care mesh validation, imputation of oral care parameters, oral care mesh generation or modification (e.g., using autoencoders, transformers, continuous normalizing flows or denoising diffusion models), metrics visualization, appliance component placement or appliance component generation or the like. In some instances, systems of this disclosure may enable a clinician or technician to process oral care data (such as scanned dental arches). In addition to segmentation, mesh cleanup, coordinate system prediction or validation operations, the systems of this disclosure may enable orthodontic treatment planning, which may involve setups prediction as at least one operation. Systems of this disclosure may also enable restoration design generation (e.g., using a denoising diffusion model), where one or more restored tooth designs are generated and processed in the course of creating oral care appliances. Systems of this disclosure may enable either or both of orthodontic or dental treatment planning, or may enable automation steps in the generation of either or both of orthodontic or dental appliances. Some appliances may enable both of dental and orthodontic treatment, while other appliances may enable one or the other.

This disclosure pertains to digital oral care, which encompasses the fields of digital dentistry and digital orthodontics. This disclosure generally describes methods of processing three-dimensional (3D) representations of oral care data. It should be understood, without loss of generality, that there are various types of 3D representations. One type of 3D representation is a 3D geometry. A 3D representation may include, be, or be part of one or more of a 3D polygon mesh, a 3D point cloud (e.g., such as derived from a 3D mesh), a 3D voxelized representation (e.g., a collection of voxels—for sparse processing), or 3D representations which are described by mathematical equations. Although the term “mesh” is used frequently throughout this disclosure, the term should be understood, in some implementations, to be interchangeable with other types of 3D representations. A 3D representation may describe elements of the 3D geometry and/or 3D structure of an object.

Dental arches S1, S2, S3 and S4 all contain the exact same tooth meshes, but those tooth meshes are transformed differently, according to the following description. A first arch S1 includes a set of tooth meshes arranged (e.g., using transforms) in their positions in the mouth, where the teeth are in the mal positions and orientations. A second arch S2 includes the same set of tooth meshes from S1 arranged (e.g., using transforms) in their positions in the mouth, where the teeth are in the ground truth setup positions and orientations. A third arch S3 includes the same meshes as S1 and S2, which are arranged (e.g., using transforms) in their positions in the mouth, where the teeth are in the predicted final setup poses (e.g., as predicted by one or more of the techniques of this disclosure). S4 is a counterpart to S3, where the teeth are in the poses corresponding to one of the several intermediate stages of orthodontic treatment with clear tray aligners.

It should be understood, without the loss of generality, that the techniques of this disclosure which apply to final setups are also applicable to intermediate staging in orthodontic treatment, particularly geometric deep learning (GDL) Setups, reinforcement learning (RL) Setups, variational autoencoder (VAE) Setups, Capsule Setups, multilayer perceptron (MLP) Setups, Diffusion Setups, pose transfer (PT) Setups, Similarity Setups, force directed graphs (FDG) Setups, Transformer Setups, Setups Comparison, or Setups Classification. The Metrics Visualization aspects of this disclosure may also be configured to visualize data from both final setups and intermediate stages. MLP Setups, VAE Setups and Capsule Setups each fall within the scope of Autoencoder Setups. Some implementations of MLP Setups may fall within the Scope of Transformer Setups. Representation Setups refers to any of MLP Setups, VAE Setups, Capsule Setups and any other setups prediction machine learning model which uses an autoencoder to create the representation for at least one tooth.

Each of the setups prediction techniques of this disclosure is applicable to the fabrication of clear tray aligners and/or indirect bonding trays. The setups predictions techniques may also be applicable to other products that involve final teeth poses, also. A pose may comprise a position (or location) and a rotation (or orientation).

A 3D mesh is a data structure which may describe the geometry or shape of an object related to oral care, including but not limited to a tooth, a hardware element, or a patient's gum tissue. A 3D mesh may include one or more mesh elements such as one or more of vertices, edges, faces and combinations thereof. In some implementations, mesh elements may include voxels, such as in the context of sparse mesh processing operations. Various spatial and structural features may be computed for these mesh elements and be provided to the predictive models of this disclosure, with the predictive models of this disclosure providing the technical advantage of improving data precision in the form of the models of this disclosure outputting more accurate predictions.

A patient's dentition may include one or more 3D representations of the patient's teeth (e.g., and/or associated transforms), gums and/or other oral anatomy. An orthodontic metric (OM) may, in some implementations, quantify the relative positions and/or orientations of at least one 3D representation of a tooth relative to at least one other 3D representation of a tooth. A restoration design metric (RDM) may, in some implementations, quantify at least one aspect of the structure and/or shape of a 3D representation of a tooth. An orthodontic landmark (OL) may, in some implementations, locate one or more points or other structural regions of interest on a 3D representation of a tooth. An OL may, in some implementations, be used in the generation of an orthodontic or dental appliance, such as a clear tray aligner or a dental restoration appliance. A mesh element may, in some implementations, comprise at least one constituent element of a 3D representation of oral care data. For example, in the case of a tooth that is represented by a 3D mesh, mesh elements may include at least: vertices, edges, faces and voxels. A mesh element feature may, in some implementations, quantify some aspect of a 3D representation in proximity to or in relation with one or more mesh elements, as described elsewhere in this disclosure. Orthodontic procedure parameters (OPP) may, in some implementations, specify at least one value which defines at least one aspect of planned orthodontic treatment for the patient (e.g., specifying desired target attributes of a final setup in final setups prediction). Orthodontic Doctor preferences (ODP) may, in some implementations, specify at least one typical value for an OPP, which may, in some instances, be derived from past cases which have been treated by one or more oral care practitioners. Restoration Design Parameters (RDP) may, in some implementations, specify at least one value which defines at least one aspect of planned dental restoration treatment for the patient (e.g., specifying desired target attributes of a tooth which is to undergo treatment with a dental restoration appliance). Doctor Restoration Design Preferences (DRDP) may, in some implementations, specify at least one typical value for an RDP, which may, in some instances, be derived from past cases which have been treated by one or more oral care practitioners. 3D oral care representations may include, but are not limited to: 1) a set of mesh element labels which may be applied to the 3D mesh elements of teeth/gums/hardware/appliance meshes (or point clouds) in the course of mesh segmentation or mesh cleanup; 2) 3D representation(s) for one or more teeth/gums/hardware/appliances for which shapes have been modified (e.g., trimmed, distorted, or filled-in) in the course of mesh segmentation or mesh cleanup; 3) one or more coordinate systems (e.g., describing one, two, three or more coordinate axes) for a single tooth or a group of teeth (such as a full arch—as with the LDE coordinate system); 4) 3D representation(s) for one or more teeth for which shapes have been modified or otherwise made suitable for use in dental restoration; 5) 3D representation(s) for one or more dental restoration appliance components; 6) one or more transforms to be applied to one or more of: dental restoration appliance library component placement relative to one or more teeth, a tooth to be placed for an orthodontic setup (either final setup or intermediate stage), a hardware element to be placed relative to one or more teeth or the like; 7) an orthodontic setup; 8) a 3D representation of a hardware element (such as facial bracket, lingual bracket, orthodontic attachment, button, hook, bite ramp, etc.) to be placed relative to one or more teeth, etc.; 8) a 3D representation of a bonding pad for a hardware element (which may be generated for a specific tooth by outlining a perimeter on the tooth, specifying a thickness to form a shell, and then subtracting-out the tooth via a Boolean operation); 9) 3D representation of a clear tray aligner (CTA); 10) the location or shape of a CTA trimline (e.g., described as either a mesh or polyline); 11) archform that describes the contours or layout of an arch of teeth (e.g., described as a 3D polyline or as a 3D mesh or surface), which may follow the incisal edges one or more teeth, which may follow the facial surfaces of one or more teeth, which may in some implementations correspond to the maloccluded arch and in other implementations correspond to the final setup arch (the effects of malocclusion on the shape of the archform may be diminished by smoothing or averaging of the shape of the archform), which may be described by one or more control points and/or a spline; 12) 3D representation of a fixture models (e.g., depictions of teeth and gums for use in thermoforming clear tray aligners, or depictions of teeth/gums/hardware for use in thermoforming indirect bonding trays); 13) one or more latent space vectors (or latent capsules) produced by the 3D encoder stage of a 3D autoencoder which has been trained on the reconstruction of oral care meshes (e.g., a variational autoencoder which has been trained for tooth reconstruction); 14) one or more oral care metrics values (e.g., such as orthodontic metrics or restoration design generation metrics) for one or more teeth; 15) one or more landmarks (e.g., 3D points) which describe the shapes and/or geometrical attributes of one or more teeth, other dentition structures or hardware structures (e.g., to be used in orthodontic setups creation or restoration appliance component generation or placement); 16) 3D representation created by scanning (e.g., optically scanning, CT scanning or MRI scanning) a 3D printed part corresponding to one or more teeth/gums/hardware/appliances (e.g., a scanned fixture model); 17) 3D printed aligners (including optionally local thickness, reinforcing rib geometry, flap positioning, or the like) 18) 3D representation of the patient's dentition that was captured chairside by a clinician or medical practitioner (e.g., in a context where the 3D representation is validated chairside, before the patient leaves the clinic, so that errors can be detected and re-scans performed as necessary); 19) dental restoration tooth design (e.g., for veneers, crowns, bridges or dental restoration appliances); 20) 3D representations of one or more teeth for use in digital oral care treatment; 21) other 3D printed parts pertaining to oral care procedures or other fields; 22) IPR cut surfaces; 23) one or more orthodontic setups transforms associated with one or more IPR cut surfaces; 24) a (digital) pontic tooth design which may fill at least a portion of the space between teeth to allow room in an orthodontic setup for an erupting tooth to later emerge from the gums; or 25) a component of a fixture model (e.g., comprising fixture model components such as interproximal webbing, block-out, bite locks, bite ramps, interproximal reinforcement, gingival ridges, torque points, power ridges, pontic tooth or dimples, among others).

The techniques of this disclosure may be advantageously combined. For example, the Setups Comparison tool may be used to compare the output of the GDL Setups model against ground truth data, compare the output of the RL Setups model against ground truth data, compare the output of the VAE Setups model against ground truth data and compare the output of the MLP Setups model against ground truth data. With each of these setups prediction models compared against ground truth data, it may be possible to determine which model gives the best performance on a certain dataset or within a given problem domain. Furthermore, the Metrics Visualization tool can enable a global view of the final setups and intermediate stages produced by one or more of the setups prediction models, with the advantage of enabling the selection of the best setups prediction model. The Metrics Visualization tool, furthermore, enables the computation of metrics which have a global scope over a set of intermediate stages. These global metrics may, in some implementations, be consumed as inputs to the neural networks for predicting setups (e.g., GDL Setups, RL Setups, VAE Setups, Capsule Setups, MLP Setups, Diffusion Setups, PT Setups, Similarity Setups, among others). The global metrics may also be provided to FDG Setups. The local metrics from this disclosure (i.e., a local metric is a metric which may be computed for one stage or setup of treatment, rather than over several stages or setups) may, in some implementations, be consumed by the neural networks herein for predicting setups, with the advantage of improving predictive results. The metrics described in this disclosure may, in some implementations, be visualized using the Metric Visualization tool.

The VAE and MAE models for mesh element labelling and mesh in-filling can be advantageously combined with the setups prediction neural networks, for the purpose of mesh cleanup ahead of or during the prediction process. In some implementations, the VAE for mesh element labelling may be used to flag mesh elements for further processing, such as metrics calculation, removal, or modification. In some instances, such flagged mesh elements may be provided as inputs to a setups prediction neural network, to inform that neural network about important mesh features, attributes or geometries, with the advantage of improving the performance of the resulting setups prediction model. In some implementations, mesh in-filling may cause the geometry of a tooth to become more nearly complete, enabling the better functioning of a setups prediction model (i.e., improved correctness of prediction on account of better-formed geometry). In some instances, a neural network to classify a setup (i.e., the Setups Classifier) may aid in the functioning of a setups prediction neural network, because the setups classifier tells that setups prediction neural network when the predicted setup is acceptable for use and can be provided to a method for aligner tray generation. A Setups Classifier (e.g., GDL Setups, RL Setups, VAE Setups, Capsule Setups, MLP Setups, Diffusion Setups, PT Setups, Similarity Setups and FDG Setups, among others) may aid in the generation of final setups and also in the generation of intermediate stages. Furthermore, a Setups Classifier neural network may be combined with the Metrics Visualization tool. In other implementations, a Setups Classification neural network may be combined with the Setups Comparison tool (e.g., the Setup Comparison tool may output an indication of how a setup produced in part by the Setups Classifier compares to a setup produced by another setups prediction method). In some implementations, the VAE for mesh element labelling may identify one or more mesh elements for use in a metrics calculation. The resulting metrics outputs may be visualized by the Metrics Visualization tool.

In some examples, the Setups Classifier neural network may aid in the setups prediction technique described in U.S. Patent Application No. US20210259808A1 (which is incorporated herein by reference in its entirety) or the setups prediction technique described in PCT Application with Publication No. WO2021245480A1 (which is incorporated herein by reference in its entirety) or in PCT Application No. PCT/IB2022/057373 (which is incorporated herein by reference in its entirety). The Setups Classifier would help one or more of those techniques to know when the predicted final setup is most nearly correct. In some instances, the Setups Classifier neural network may output an indication of how far away from final setup a given setup is (i.e., a progress indicator).

In some implementations, the latent space embedding vector(s) from the reconstruction VAE can be concatenated with the inputs to the setups prediction neural network described in WO2021245480A1. The latent space vectors can also be incorporated as inputs to the other setups prediction models: GDL Setups, RL Setups, VAE Setups, Capsule Setups, MLP Setups and Diffusion Setups, among others. The advantage is to impart the reconstruction characteristics (e.g., latent vector dimensions of a tooth mesh) to that neural network, hence improving the generated setups prediction.

In some examples, the various setups prediction neural networks of this disclosure may work together to produce the setups required for orthodontic treatment. For example, the GDL Setups model may produce a final setup, and the RL Setups model may use that final setup as input to produce a series of intermediate stages setups. Alternatively, the VAE Setups model (or the MLP Setups model) may create a final setup which may be used by an RL Setups model to produce a series of intermediate stages setups. In some implementations, a setup prediction may be produced by one setups prediction neural network, and then taken as input to another setups prediction neural network for further improvements and adjustments to be made. In some implementations, such improvements may be performed in iterative fashion.

In some implementations, a setups validation model, such as the model disclosed in U.S. Provisional Application No. 63/366,495, may be involved in this iterative setups prediction loop. First a setup may be generated (e.g., using a model trained for setups prediction, such as GDL Setups, RL Setups, VAE Setups, Capsule Setups, MLP Setups, Diffusion Setups, PT Setups, Similarity Setups and FDG Setups, among others), then the setup undergoes validation. If the setup passes validation, the setup may be outputted for use. If the setup fails validation, the setup may be sent back to one or more of the setups prediction models for corrections, improvements and/or adjustments. In some instances, the setups validation model may output an indication of what is wrong with the setup, enabling the setups generation model to make an improved version upon the next iteration. The process iterates until done.

Generally speaking, in some implementations, two or more of the following techniques of the present disclosure may be combined in the course of orthodontic and/or dental treatment: GDL Setups, Setups Classification, Reinforcement Learning (RL) Setups, Setups Comparison, Autoencoder Setups (VAE Setups or Capsule Setups), VAE Mesh Element Labeling, Masked Autoencoder (MAE) Mesh In-filling, Multi-Layer Perceptron (MLP) Setups, Metrics Visualization, Imputation of Missing Oral Care Parameters Values, Tooth Classification Using Latent Vector, FDG Setups, Pose Transfer Setups, Restoration Design Metrics Calculation, Neural Network Techniques for Dental Restoration and/or Orthodontics (e.g., 3D Oral Care Representation Generation or Modification Using Transformers), Landmark-based (LB) Setups, Diffusion Setups, Imputation of Tooth Movement Procedures, Capsule Autoencoder Segmentation, Diffusion Segmentation, Similarity Setups, Validation of Oral Care Representations (e.g., using autoencoders), Coordinate System Prediction, Restoration Design Generation, or Geometry Generation (or Modification) Using Denoising Diffusion Models.

Oral care parameters may include one or more values that specify orthodontic procedure parameters, or restoration design parameters (RDP), as described herein. Oral care parameters may define one or more intended aspects of a 3D oral care representation and may be provided to an ML model to promote that ML model to generate output which may be used in the generation of oral care appliances that are suitable for the treatment of a patient. Other types of values include doctor preferences and restoration design preferences, as described herein. Doctor preferences and restoration design preferences may define the typical treatment choices or practices of a particular clinician. Restoration design preferences are subjective to a particular clinician, and so differ from restoration design parameters. In some implementations, doctor preferences or restoration design preferences may be computed by unsupervised means, such as clustering, which may determine the typical values that a clinician uses in patient treatment. Those typical values may be stored in a datastore and recalled to be provided to an automated ML model as default values (e.g., default values which may be modified before execution of the model).

For example, one clinician may prefer one value for a restoration design parameter (RDP), while another clinician may prefer a different value for that RDP, when faced with a similar diagnosis or treatment protocol. One example of such an RDP is dental restoration style. Procedure parameters and/or doctor preferences may, in some implementations, be provided to a setups prediction model for orthodontic treatment, for the purpose of improving the customization of the resulting orthodontic appliance. Restoration design parameters and doctor restoration preferences may in some implementations be used to design tooth geometry for use in the creation of a dental restoration appliance, for the purpose of improving the customization of that appliance. In addition to oral care parameters, doctor preferences, and doctor restoration preferences, some implementations of ML prediction models of this disclosure, in orthodontic treatment, may also take as input a setup (e.g., an arrangement of teeth). In some such implementations, an ML prediction model of this disclosure may take as input a final setup (i.e., final arrangement of teeth), such as in the case of a prediction model trained to generate intermediate stages. For simplicity, these preferences are referred to as doctor restoration preferences, but it is intended to be used in a non-limiting sense. Specifically, it should be appreciated that these preferences may be specified by any treating or otherwise appropriate medical professional and are not intended to be limited to doctor preferences per se (i.e., preferences from someone in possession of an M. D. or equivalent degree).

An oral care professional or clinician, such as a dentist or orthodontist, may specify information about patient treatment in the form of a patient-specific set of procedure parameters. In some instances, an oral care professional may specify a set of general preferences (aka doctor preferences) for use over a broad range of cases, to use as default values in the set of procedure parameters specification process. Oral care parameters may in some implementations be incorporated into the techniques described in this disclosure, such as one or more of GDL Setups, VAE Setups, RL Setups, Setups Comparison, Setups Classification, VAE Mesh Element Labelling, MAE Mesh In-Filling, Validation Using Autoencoders, Imputation of Missing Procedure Parameters Values, Metrics Visualization, or FDG Setups. One or more of these models may take as input one or more procedure parameters vector K and/or one or more doctor preference vectors L. In some implementations, one or more of these models may introduce to one or more of a neural network's hidden layers one or more procedure parameters vector K and/or one or more doctor preferences vectors L. In some implementations, one or more of these models may introduce either or both of K and L to a mathematical calculation, such as a force calculation, for the purpose of improving that calculation and the ultimate customization of the resulting appliance to the patient.

Some implementations of a neural network for predicting a setup (such as GDL Setup, VAE Setup or RL Setup) may incorporate information from an oral care professional (aka doctor). This information may influence the arrangement of teeth in the final setup, bringing the positions and orientations of the teeth into conformance with a specification set by the doctor, within tolerances. In some implementations of the GDL Setup model, oral care parameters may be fed directly into the generator network as a separate input alongside the mesh data. In some implementations of GDL Setups, oral care parameters may be incorporated into the feature vector which is computed for each mesh element before the mesh elements are provided to the generator for processing. Some implementations of a setups prediction model (e.g., VAE setups or diffusion setups) may incorporate oral care parameters into the setups predictions. In some implementations, the procedure parameters K and/or the doctor preference information L may be concatenated with the latent space vector C. A doctor's preferences (e.g., in an orthodontic context) and/or doctor's restoration preferences may be indicated in a treatment form, or they could be based upon characteristics in treatment plans such as final setup characteristics (e.g., amount of bite correction or midline correction in planned final setups), intermediate staging characteristics (e.g., treatment duration, tooth movement protocols, or overcorrection strategies), or outcomes (e.g., number of revisions/refinements).

Orthodontic procedure parameters may specify one or more of the following (with possible values shown in { }). Non-limiting categorical values for some example OPP are described below. In some implementations, a real value may be specified for one or more of these OPP. For example, the Overbite OPP may specify a quantity of overbite (e.g., in millimeters) which is desired in a setup, and may be received as input of a setups prediction model to provide that setups prediction model information about the amount of overbite which is desired in the setup. Some implementations may specify a numerical value for the Overjet OPP, or other OPP. In some implementations, one or more OPP may be defined which correspond to one or more orthodontic metrics (OM). In some instances, a numerical value may be specified for such an OPP, for the purpose of controlling the output of a setups prediction model.

```
Teeth To Move: {AnteriorsOnly, AnteriorsAndBicuspids, FullArch}
Tooth Movement Restrictions: for each tooth, indicate if tooth is {DoNotMove, Missing, ToBeExtracted,
Primary/Erupting, Clear}
Overbite: {ShowResultingOverbiteAfterAlignment, MaintainInitialOverbite, CorrectOpenBite,
    CorrectDeepBite}
Overjet: {ShowResultingOverjetAfterAlignment, MaintainInitialOverjet, ImproveResultingOverjet}
Anterior/Posterior (AP) Relationship
  Maintain: {Right, Left, Both}
  Improve canine relationship only: {Right, Left, Both}
  Improve canine and/or molar relationship up to 4mm: {Right, Left, Both}
  Correct to Class I (canine and molar): {Right, Left, Both}
Crossbite (if present)
  Anterior: {DoNotCorrect, Correct, N/A}
  Posterior: {DoNotCorrect, Correct, N/A}
Correction to Class I (canine and molar): {Right, Left, Both}
  Correct with Posterior IPR: {yes, no}
      Class II/III correction simulation (elastics required): {yes, no}
      Sequential Distalization (elastic recommended): {yes, no}
      Include cuts for elastics?: {yes, no}
      Preferred cuts for elastics: {UseButtonCutoutsOnMolarsAndHooksOnCanines,
        UseButtonCutoutsOnly, UseHooksOnly}
      Stage to start cuts for elastics: [integer]
LevelingOfUpperAnteriors: {Laterals0.5mmShorterThanCentral, LevelIncisalEdges,
LevelGingivalMargins, AsIndicated}
Spacing: {CloseAllSpaces, LeaveSpecificSpaces}
Preferred Midline Position: {SetTheUpperMidlineToIdeal, MatchTheUpperAndLowerToEachOther}
Resolve Upper Crowding by Expand: {Primarily, AsNeeded, None}
Resolve Upper Crowding by Procline: {Primarily, AsNeeded, None}
Resolve Upper Crowding by IPR - Anterior: {Primarily, AsNeeded, None}
Resolve Upper Crowding by IPR - Posterior Right: {Primarily, AsNeeded, None}
Resolve Upper Crowding by IPR - Posterior Left: {Primarily, AsNeeded, None}
Resolve Lower Crowding by Expand: {Primarily, AsNeeded, None}
Resolve Lower Crowding by Procline: {Primarily, AsNeeded, None}
Resolve Lower Crowding by IPR - Anterior: {Primarily, AsNeeded, None}
Resolve Lower Crowding by IPR - Posterior Right: {Primarily, AsNeeded, None}
Resolve Lower Crowding by IPR - Posterior Left: {Primarily, AsNeeded, None}
Finishing Arch Form: {Patient'sNatural, AsIndicated}
  [doctor can specify an archform - selected from a set of options or custom-designed]
```

Other orthodontic procedure parameters may be defined, such as those which may be used to place standardized brackets at prescribed occlusal heights on the teeth. In some implementations, one or more orthodontic procedure parameters may be defined to specify at least one of the 2^{nd} and 3^{rd} order rotation angles to be applied to a tooth (i.e., angulation and torque, respectively), which may enable a target setup arrangement where crown landmarks lie within a threshold distance of a common occlusal plane, for example. In some implementations, one or more orthodontic procedure parameters may be defined to specify the position in global coordinates where at least one landmark (e.g., a centroid) of a tooth crown (or root) is to be placed in a setup arrangement of teeth. Generally, an oral care parameter may be defined which corresponds to an oral care metric. For example, an orthodontic procedure parameter may be defined which corresponds to an orthodontic metric (e.g., to specify at the input of a setups prediction model an amount of a certain metric which is desired to appear in a predicted setup).

Doctor preferences may differ from orthodontic procedure parameters in that doctor preferences pertain to an oral care provider and may comprise of the means, modes, medians, minimums, or maximums (or some other statistic) of past settings associated with an oral care provider's treatment decisions on past orthodontic cases. Procedure parameters, on the other hand, may pertain to a specific patient, and describe the needs of a particular patient's treatment. Doctor preferences may pertain to a doctor and the doctor's past treatment practices, whereas procedure parameters may pertain to the treatment of a particular patient. Doctor preferences (or “treatment preferences”) may specify one or more of the following (with some non-limiting possible values shown in { }). Other possible values are found elsewhere in this disclosure.

Doctor preferences may specify one or more of the following.

```
Deep Bite Cases (Amount of Bite Correction) - Final Overbite: [real value in millimeters, e.g., 0.5 mm]
  Option - Intrude Upper Anteriors: {yes, no}
  Option - Include lower canines in vertical overcorrection: {yes, no}
Midline Correction in Planned Final Setup: {MaintainInitialMidline, ImproveMidlineWithIPR,
AsIndicated}
Deep Bite Cases - Reverse Curve of Speed: {yes, no}
Anterior Open Bite Cases - Final Overbite: [real value in millimeters, e.g., 2 mm]
Is Arch Expansion a Priority for Your Cases?: {Yes, No}
  If yes, specify acceptable expansion per quadrant in mm.
  When expanding upper molars, apply buccal root torque: {yes, no}
Is IPR Acceptable of First Tx Design: {yes, no}
Maximum IPR per contact:
  Upper Anterior: [specify in mm]
  Lower Anterior: [specify in mm]
  Upper and Lower Anterior: [specify in mm]
Is Asymmetric IPR Acceptable?: {yes, no}
Final Tooth Position (Overcorrection Strategy): {Ideal, Overcorrected}
Root Movement: {MoveRootsAsNeededToAchieveTreatmentGoals, LimitPosteriorRootMovement,
LimitAllRootMovement}
Final Occlusal Contacts: {AllContactsBalancedWhenPossible, NoOcclusalContactOnUpperIncisors,
FinishWithHeavyPosteriorContacts, Other}
Is Asymmetric AP Shift Acceptable for Class Correction?: {yes, no, other}
Treatment Duration: [count of stages]
Tooth Movement Protocol: {protocol_A, protocol_B, protocol_C}
```

Existing techniques have attempted to move the teeth towards an archform V after a setup prediction has already been rendered by other workflow components, which introduces error to the resulting setup and diminishes performance in view of the purpose of the workflow components which made the setups prediction. The present disclosure provides several improvements over these existing techniques by enabling archform information to be introduced directly into the setups prediction neural network as an input to that neural network, with the technical improvement of providing setups predictions that more accurately meets the orthodontic treatment needs of the patient (thereby improving data precision). Archform information V may be provided as an input to any of the GDL Setups, RL Setups, VAE Setups, Capsule Setups, MLP Setups and Diffusion Setups prediction neural networks. In some implementations, archform information V may be provided directly to one or more internal neural network layers in one or more of those setups applications.

The additional procedure parameters may include text descriptions of the patient's medical condition and of the intended treatment. Such text descriptions may be analyzed via natural language processing operations, including tokenization, stop word removal, stemming, n-gram formation, text data vectorization, bag of words analysis, term frequency inverse document frequency (TF-IDF) analysis, sentiment analysis, naive Bayes classification, and/or logistic regression classification. The outputs of such analysis techniques may be used as input to one or more of the neural networks of this disclosure with the advantage of customizing and improving the predicted outputs (e.g., the predicted setups or predicted mesh geometries).

These additional orthodontic parameters and doctor preferences may also be incorporated into the neural networks of this disclosure, with the data precision and efficiency-related advantages of improving the customization of those neural networks and enabling those neural networks to predict output examples which better match the treatment needs of the individual patient.

In some implementations, a dataset used for training one or more of the neural network models of this disclosure may be filtered conditionally on one or more of the orthodontic procedure parameters described in this section. In some instances, patient cases which exhibit outlier values for one or more of these procedure parameters may be omitted from a dataset (alternatively used to form a dataset) for training one or more of the neural networks of this disclosure.

One or more procedure parameters and/or doctor preferences may be provided to a neural network during training. In this manner the neural network may be conditioned on the one or more procedure parameters and/or doctor preferences. Examples of such neural networks include a conditional generative adversarial network (cGAN) and/or a conditional variational autoencoder (cVAE), either of which may be used for the various neural network-based applications of this disclosure.

In some instances, tooth shape-based inputs may be provided to a neural network for setups predictions. In other instances, non-shape-based inputs can be used, such as a tooth name or designation, as it pertains to dental notation. In some implementations, a vector R of flags may be provided to the neural network, where a ‘1’ value indicates that the tooth is present and a ‘0’ value indicates that the tooth is absent from the patient case (though other values are possible). The vector R may comprise a 1-hot vector, where each element in the vector corresponds to a tooth type, name or designation. Identifying information about a tooth (e.g., the tooth's name) can be provided to the predictive neural networks of this disclosure, with the advantage of enabling the neural network to become trained to handle different teeth in tooth-specific ways. For example, the setups prediction model may learn to make setups transformations predictions for a specific tooth designation (e.g., upper right central incisor, or lower left cuspid, etc.). In the case of the mesh cleanup autoencoders (either for labelling mesh element or for in-filling missing mesh data), the autoencoder may be trained to provide specialized treatment to a tooth according to that tooth's designation, in this manner. In the case of a setups classification neural network, a listing of tooth name(s) present in the patient's arch may better enable the neural network to output an accurate determination of setup classification, because tooth designation is a valuable input to training such a neural network. Tooth designation/name may be defined, for example, according to the Universal Numbering System, Palmer System, or the FDI World Dental Federation notation (ISO 3950).

In one example, where all except the (up to four) wisdom teeth are present in the case, a vector R may be defined as an optional input to the setups prediction neural networks of this disclosure, where there is a 0 in the vector element corresponding to each of the wisdom teeth, and a 1 in the elements corresponding to the following teeth: UR7, UR6, UR5, UR4, UR3, UR2, UR1, UL1, UL2, UL3, UL4, UL5, UL6, UL7, LL7, LL6, LL5, LL4, LL3, LL2, LL1, LR1, LR2, LR3, LR4, LR5, LR6, LR7

In some instances, the position of the tooth tip may be provided to a neural network for setups predictions. In other instances, one or more vectors S of the orthodontic metrics described elsewhere in this disclosure may be provided to a neural network for setups predictions. The advantage is an improved capacity for the network to become trained to understand the state of a maloccluded setup and therefore be able to predict a more accurate final setup or intermediate stage.

In some implementations, the neural networks may take as input one or more indications of interproximal reduction (IPR) U, which may indicate the amount of enamel that is to be removed from a tooth during the course orthodontic treatment (either mesially or distally). In some implementations, IPR information (e.g., quantity of IPR that is to be performed on one or more teeth, as measured in millimeters, or one or more binary flags to indicate whether or not IPR is to be performed on each tooth identified by flagging) may be concatenated with a latent vector A which is produced by a VAE or a latent capsule T autoencoder. The vector(s) and/or capsule(s) resulting from such a concatenation may be provided to one or more of the neural networks of the present disclosure, with the technical improvement or added advantage of enabling that predictive neural network to account for IPR. IPR is especially relevant to setups prediction methods, which may determine the positions and poses of teeth at the end of treatment or during one or more stages during treatment. It is important to account for the amount of enamel that is to be removed ahead of predicted tooth movements.

In some implementations, one or more procedure parameters K and/or doctor preferences vectors L may be introduced to a setups prediction model. In some implementations, one or more optional vectors or values of tooth position N (e.g., XYZ coordinates, in either tooth local or global coordinates), tooth orientation O (e.g., pose, such as in transformation matrices or quaternions, Euler angles or other forms described herein), dimensions of teeth P (e.g., length, width, height, circumference, diameter, diagonal measure, volume-any of which dimensions may be normalized in comparison to another tooth or teeth), distance between adjacent teeth Q. These “dimensions of teeth P” may in some instances be used to describe the intended dimensions of a tooth for dental restoration design generation.

In some implementations, tooth dimensions P (e.g., length, width, height, or circumference) may be measured inside a plane, such as the plane that intersects the centroid of the tooth, or the plane that intersects a center point that is located midway between the centroid and either the incisal-most extent or the gingival-most extent of the tooth. The tooth dimension of height may be measured as the distance from gums to incisal edge. The tooth dimension of width may be measured as the distance from the mesial extent to the distal extent of the tooth. In some implementations, the circularity or roundness of the tooth cross-section may be measured and included in the vector P. Circularity or roundness may be defined as the ratio of the radii of inscribed and circumscribed circles.

The distance Q between adjacent teeth can be implemented in different ways (and computed using different distance definitions, such as Euclidean or geodesic). In some implementations, a distance Q1 may be measured as an averaged distance between the mesh elements of two adjacent teeth. In some implementations, a distance Q2 may be measured as the distance between the centers or centroids of two adjacent teeth. In some implementations, a distance Q3 may be measured between the mesh elements of closest approach between two adjacent teeth. In some implementations, a distance Q4 may be measured between the cusp tips of two adjacent teeth. Teeth may, in some implementations, be considered adjacent within an arch. Teeth may, in some implementations, also be considered adjacent between opposing arches. In some implementations, any of Q1, Q2, Q3 and Q4 may be divided by a term for the purpose of normalizing the resulting value of Q. In some implementations, the normalizing term may involve one or more of: the volume of a tooth, the count of mesh elements in a tooth, the surface area of a tooth, the cross-sectional area of a tooth (e.g., as projected into the XY plane), or some other term related to tooth size.

Other information about the patient's dentition or treatment needs (or related parameters) may be concatenated with the other input vectors to one or more of MLP, GAN, generator, encoder structure, decoder structure, transformer, VAE, conditional VAE, regularized VAE, 3D U-Net, capsule autoencoder, diffusion model, and/or any of the neural networks models listed elsewhere in this disclosure.

The vector M may contain flags which apply to one or more teeth. In some implementations, M contains at least one flag for each tooth to indicate whether the tooth is pinned. In some implementations, M contains at least one flag for each tooth to indicate whether the tooth is fixed. In some implementations, M contains at least one flag for each tooth to indicate whether the tooth is pontic. Other and additional flags are possible for teeth, as are combinations of fixed, pinned and pontic flags. A flag that is set to a value that indicates that a tooth should be fixed is a signal to the network that the tooth should not move over the course of treatment. In some implementations, the neural network loss function may be designed to be penalized for any movement in the indicated teeth (and in some particular cases, may be heavily penalized). A flag to indicate that a tooth is pontic informs the network that the tooth gap is to be maintained, although that gap is allowed to move. In some cases, M may contain a flag indicating that a tooth is missing. In some implementations, the presence of one or more fixed teeth in an arch may aid in setups prediction, because the one or more fixed teeth may provide an anchor for the poses of the other teeth in the arch (i.e., may provide a fixed reference for the pose transformations of one or more of the other teeth in the arch). In some implementations, one or more teeth may be intentionally fixed, so as to provide an anchor against which the other teeth may be positioned. In some implementations, a 3D representation (such as a mesh) which corresponds to the gums may be introduced, to provide a reference point against which teeth can be moved.

Without the loss of generality, one or more of the optional input vectors K, L, M, N, O, P, Q, R, S, U and V described elsewhere in this disclosure may also be provided to the input or into an intermediate layer of one or more of the predictive models of this disclosure. In particular, these optional vectors may be provided to the MLP Setups, GDL Setups, RL Setups, VAE Setups, Capsule Setups and/or Diffusion Setups, with the advantage of enabling the respective model to generate setups which better meet the orthodontic treatment needs of the patient. In some implementations, such inputs may be provided, for example, by being concatenated with one or more latent vectors A which are also provided to one or more of the predictive models of this disclosure. In some implementations, such inputs may be introduced, for example, by being concatenated with one or more latent capsules T which are also provided to one or more of the predictive models of this disclosure.

In some implementations, one or more of K, L, M, N, O, P, Q, R, S, U and V may be introduced to the neural network (e.g., MLP or Transformer) directly in a hidden layer of the network. In some instances, one or more of K, L, M, N, O, P, Q, R, S, U and V may be introduced directly into the internal processing of an encoder structure.

In some implementations, a setups prediction model (such as GDL Setups, RL Setups, VAE Setups, Capsule Setups, MLP Setups, PT Setups, Similarity Setups and Diffusion Setups) may take as input one or more latent vectors A which correspond to one or more input oral care meshes (e.g., such as tooth meshes). In some implementations, a setups prediction model (such as GDL Setups, RL Setups, VAE Setups, Capsule Setups, MLP Setups and Diffusion Setups) may take as input one or more latent capsules T which correspond to one or more input oral care meshes (e.g., such as tooth meshes). In some implementations, a setups prediction method may take as input both of A and T.

Various loss calculation techniques are generally applicable to the techniques of this disclosure (e.g., GDL Setups, RL Setups, VAE Setups, Capsule Setups, MLP Setups, Diffusion Setups, PT Setups, Similarity Setups, Setups Classification, Tooth Classification, VAE Mesh Element Labelling, MAE Mesh In-Filling and the imputation of procedure parameters).

These losses include L1 loss, L2 loss, mean squared error (MSE) loss, cross entropy loss, among others. Losses may be computed and used in the training of neural networks, such as multi-layer perceptron's (MLP), U-Net structures, generators and discriminators (e.g., for GANs), autoencoders, variational autoencoders, regularized autoencoders, masked autoencoders, transformer structures, or the like. Some implementations may use either triplet loss or contrastive loss, for example, in the learning of sequences.

Losses may also be used to train encoder structures and decoder structures. A KL-Divergence loss may be used, at least in part, to train one or more of the neural networks of the present disclosure, such as a mesh reconstruction autoencoder or the generator of GDL Setups, which the advantage of imparting Gaussian behavior to the optimization space. This Gaussian behavior may enable a reconstruction autoencoder to produce a better reconstruction (e.g., when a latent vector representation is modified and that modified latent vector is reconstructed using a decoder, the resulting reconstruction is more likely to be a valid instance of the inputted representation). There are other techniques for computing losses which may be described elsewhere in this disclosure. Such losses may be based on quantifying the difference between two or more 3D representations.

MSE loss calculation may involve the calculation of an average squared distance between two sets, vectors or datasets. MSE may be generally minimized. MSE may be applicable to a regression problem, where the prediction generated by the neural network or other machine learning model may be a real number. In some implementations, a neural network may be equipped with one or more linear activation units on the output to generate an MSE prediction. Mean absolute error (MAE) loss and mean absolute percentage error (MAPE) loss can also be used in accordance with the techniques of this disclosure.

Cross entropy may, in some implementations, be used to quantify the difference between two or more distributions. Cross entropy loss may, in some implementations, be used to train the neural networks of the present disclosure. Cross entropy loss may, in some implementations, involve comparing a predicted probability to a ground truth probability. Other names of cross entropy loss include “logarithmic loss,” “logistic loss,” and “log loss”. A small cross entropy loss may indicate a better (e.g., more accurate) model. Cross entropy loss may be logarithmic. Cross entropy loss may, in some implementations, be applied to binary classification problems. In some implementations, a neural network may be equipped with a sigmoid activation unit at the output to generate a probability prediction. In the case of multi-class classifications, cross entropy may also be used. In such a case, a neural network trained to make multi-class predictions may, in some implementations, be equipped with one or more softmax activation functions at the output (e.g., where there is one output node for class that is to be predicted). Other loss calculation techniques which may be applied in the training of the neural networks of this disclosure include one or more of: Huber loss, Hinge loss, Categorical hinge loss, cosine similarity, Poisson loss, Logcosh loss, or mean squared logarithmic error loss (MSLE). Other loss calculation methods are described herein and may be applied to the training of any of the neural networks described in the present disclosure.

One or more of the neural networks of the present disclosure may, in some implementations, be trained, at least in part by a loss which is based on at least one of: a Point-wise Mesh Euclidean Distance (PMD) and an Earth Mover's Distance (EMD). Some implementations may incorporate a Hausdorff Distance (HD) calculation into the loss calculation. Computing the Hausdorff distance between two or more 3D representations (such as 3D meshes) may provide one or more technical improvements, in that the HD not only accounts for the distances between two meshes, but also accounts for the way that those meshes are oriented, and the relationship between the mesh shapes in those orientations (or positions or poses). Hausdorff distance may improve the comparison of two or more tooth meshes, such as two or more instances of a tooth mesh which are in different poses (e.g., such as the comparison of predicted setup to ground truth setup which may be performed in the course of computing a loss value for training a setups prediction neural network).

Reconstruction loss may compare a predicted output to a ground truth (or reference) output. Systems of this disclosure may compute reconstruction loss as a combination of L1 loss and MSE loss, as shown in the following line of pseudocode: reconstruction loss=0.5*L1 (all_points_target, all_points_predicted)+0.5*MSE (all_points_target, all_points_predicted). In the above example, all_points_target is a 3D representation (e.g., a 3D mesh or point cloud) corresponding to ground truth data (e.g., a ground truth tooth restoration design, or a ground truth example of some other 3D oral care representation). In the above example, all_points_predicted is a 3D representation (e.g., a 3D mesh or point cloud) corresponding to generated or predicted data (e.g., a generated tooth restoration design, or a generated example of some other kind of 3D oral care representation). Other implementations of reconstruction loss may additionally (or alternatively) involve L2 loss, mean absolute error (MAE) loss or Huber loss terms.

Reconstruction error may compare reconstructed output data (e.g., as generated by a reconstruction autoencoder, such as a tooth design which has been generated for use in generating a dental restoration appliance) to the original input data (e.g., the data which were provided to the input of the reconstruction autoencoder, such as a pre-restoration tooth). Systems of this disclosure may compute reconstruction error as a combination of L1 loss and MSE loss, as shown in the following line of pseudocode: reconstruction_error=0.5*L1 (all_points_input, all_points_reconstructed)+0.5*MSE (all_points_input, all_points_reconstructed). In the above example, all_points_input is a 3D representation (e.g., a 3D mesh or point cloud) corresponding to input data (e.g., the pre-restoration tooth design, which was provided to a reconstruction autoencoder, or another 3D oral care representation which is provided to the input of an ML model). In the above example, all_points_reconstructed is a 3D representation (e.g., 3D mesh or point cloud) corresponding to reconstructed (or generated) data (e.g., a reconstructed tooth restoration design, or another example of a generated 3D oral care representation).

In other words, reconstruction loss is concerned with computing a difference between a predicted output and a reference output, whereas reconstruction error is concerned with computing a difference between a reconstructed output and an original input from which the reconstructed data are derived.

The techniques of this disclosure may include operations such as 3D convolution, 3D pooling, 3D unconvolution and 3D unpooling. 3D convolution may aid segmentation processing, for example in down sampling a 3D mesh. 3D un-convolution undoes 3D convolution, for example, in a U-Net. 3D pooling may aid the segmentation processing, for example in summarized neural network feature maps. 3D un-pooling undoes 3D pooling, for example in a U-Net. These operations may be implemented by way of one or more layers in the predictive or generative neural networks described herein. These operations may be applied directly on mesh elements, such as mesh edges or mesh faces. These operations provide for technical improvements over other approaches because the operations are invariant to mesh rotation, scale, and translation changes. In general, these operations depend on edge (or face) connectivity, therefore these operations remain invariant to mesh changes in 3D space as long as edge (or face) connectivity is preserved. That is, the operations may be applied to an oral care mesh and produce the same output regardless of the orientation, position or scale of that oral care mesh which may lead to data precision improvement. MeshCNN is a general-purpose deep neural network library for 3D triangular meshes, which can be used for tasks such as 3D shape classification or mesh element labelling (e.g., for segmentation or mesh cleanup). MeshCNN implements these operations on mesh edges. Other toolkits and implementations may operate on edges or faces.

In some implementations of the techniques of this disclosure, neural networks may be trained to operate on 2D representations (such as images). In some implementations of the techniques of this disclosure, neural networks may be trained to operate on 3D representations (such as meshes or point clouds). An intraoral scanner may capture 2D images of the patient's dentition from various views. An intraoral scanner may also (or alternatively) capture 3D mesh or 3D point cloud data which describes the patient's dentition. According to various techniques, autoencoders (or other neural networks described herein) may be trained to operate on either or both of 2D representations and 3D representations.

A 2D autoencoder (comprising a 2D encoder and a 2D decoder) may be trained on 2D image data to encode an input 2D image into a latent form (such as a latent vector or a latent capsule) using the 2D encoder, and then reconstruct a facsimile of the input 2D image using the 2D decoder. In the case of a handheld mobile app which has been developed for such analysis (e.g., for the analysis of dental anatomy), 2D images may be readily captured using one or more of the onboard cameras. In other examples, 2D images may be captured using an intraoral scanner which is configured for such a function. Among the operations which may be used in the implementation a 2D autoencoder (or other 2D neural network) for 2D image analysis are 2D convolution, 2D pooling and 2D reconstruction error calculation.

2D image convolution may involve the “sliding” of a kernel across a 2D image and the calculation of elementwise multiplications and the summing of those elementwise multiplications into an output pixel. The output pixel that results from each new position of the kernel is saved into an output 2D feature matrix. In some implementations, neighboring elements (e.g., pixels) may be in well-defined locations (e.g., above, below, left and right) in a rectilinear grid.

A 2D pooling layer may be used to down sample a feature map and summarize the presence of certain features in that feature map.

2D reconstruction error may be computed between the pixels of the input and reconstructed images. The mapping between pixels may be well understood (e.g., the upper pixel [23,134] of the input image is directly compared to pixel [23,134] of the reconstructed image, assuming both images have the same dimensions).

Among the advantages provided by the 2D autoencoder-based techniques of this disclosure is the ease of capturing 2D image data with a handheld device. In some instances, where outside data sources provide the data for analysis, there may be instances where only 2D image data are available. When only 2D image data are available, then analysis using a 2D autoencoder is warranted.

Modern mobile devices (such as commercially available smartphones) may also have the capability of generating 3D data (e.g., using multiple cameras and stereophotogrammetry, or one camera which is moved around the subject to capture multiple images from different views, or both), which in some implementations, may be arranged into 3D representations such as 3D meshes, 3D point clouds and/or 3D voxelized representations. The analysis of a 3D representation of the subject may in some instances provide technical improvements over 2D analysis of the same subject. For example, a 3D representation may describe the geometry and/or structure of the subject with less ambiguity than a 2D representation (which may contain shadows and other artifacts which complicate the depiction of depth from the subject and texture of the subject). In some implementations, 3D processing may enable technical improvements because of the inverse optics problem which may, in some instances, affect 2D representations. The inverse optics problem refers to the phenomenon where, in some instances, the size of a subject, the orientation of the subject and the distance between the subject and the imaging device may be conflated in a 2D image of that subject. Any given projection of the subject on the imaging sensor could map to an infinite count of {size, orientation, distance} pairings. 3D representations enable the technical improvement in that 3D representations remove the ambiguities introduced by the inverse optics problem.

A device that is configured with the dedicated purpose of 3D scanning, such as a 3D intraoral scanner (or a CT scanner or MRI scanner), may generate 3D representations of the subject (e.g., the patient's dentition) which have significantly higher fidelity and precision than is possible with a handheld device. When such high-fidelity 3D data are available (e.g., in the application of oral care mesh classification or other 3D techniques described herein), the use of a 3D autoencoder is offers technical improvements (such as increased data precision), to extract the best possible signal out of those 3D data (i.e., to get the signal out of the 3D crown meshes used in tooth classification or setups classification).

A 3D autoencoder (comprising a 3D encoder and a 3D decoder) may be trained on 3D data representations to encode an input 3D representation into a latent form (such as a latent vector or a latent capsule) using the 3D encoder, and then reconstruct a facsimile of the input 3D representation using the 3D decoder. Among the operations which may be used to implement a 3D autoencoder for the analysis of a 3D representation (e.g., 3D mesh or 3D point cloud) are 3D convolution, 3D pooling and 3D reconstruction error calculation.

For each mesh element, a 3D convolution may be performed to aggregate local features from nearby mesh elements. Processing may be performed above and beyond the techniques for 2D convolution, to account for the differing count and locations of neighboring mesh elements (relative to a particular mesh element). A particular 3D mesh element may have a variable count of neighbors and those neighbors may not be found in expected locations (as opposed to a pixel in 2D convolution which may have a fixed count of neighboring pixels which may be found in known or expected locations). In some instances, the order of neighboring mesh elements may be relevant to 3D convolution.

A 3D pooling operation may enable the combining of features from a 3D mesh (or other 3D representation) at multiple scales. 3D pooling may iteratively reduce a 3D mesh into mesh elements which are most highly relevant to a given application (e.g., for which a neural network has been trained). Similarly to 3D convolution, 3D pooling may benefit from special processing beyond that entailed in 2D convolution, to account for the differing count and locations of neighboring mesh elements (relative to a particular mesh element). In some instances, the order of neighboring mesh elements may be less relevant to 3D pooling than to 3D convolution.

3D reconstruction error may be computed using one or more of the techniques described herein, such as computing Euclidean distances between corresponding mesh elements, between the two meshes. Other techniques are possible in accordance with aspects of this disclosure. 3D reconstruction error may generally be computed on 3D mesh elements, rather than the 2D pixels of 2D reconstruction error. 3D reconstruction error may enable technical improvements over 2D reconstruction error, because a 3D representation may, in some instances, have less ambiguity than a 2D representation (i.e., have less ambiguity in form, shape and/or structure). Additional processing may, in some implementations, be entailed for 3D reconstruction which is above and beyond that of 2D reconstruction, because of the complexity of mapping between the input and reconstructed mesh elements (i.e., the input and reconstructed meshes may have different mesh element counts, and there may be a less clear mapping between mesh elements than there is for the mapping between pixels in 2D reconstruction). The technical improvements of 3D reconstruction error calculation include data precision improvement.

A 3D representation may be produced using a 3D scanner, such as an intraoral scanner, a computerized tomography (CT) scanner, ultrasound scanner, a magnetic resonance imaging (MRI) machine or a mobile device which is enabled to perform stereophotogrammetry. A 3D representation may describe the shape and/or structure of a subject. A 3D representation may include one or more 3D mesh, 3D point cloud, and/or a 3D voxelized representation, among others. A 3D mesh includes edges, vertices, or faces. Though interrelated in some instances, these three types of data are distinct. The vertices are the points in 3D space that define the boundaries of the mesh. These points would alternatively be described as a point cloud but for the additional information about how the points are connected to each other, as described by the edges. An edge is described by two points and can also be referred to as a line segment. A face is described by a number of edges and vertices. For instance, in the case of a triangle mesh, a face comprises three vertices, where the vertices are interconnected to form three contiguous edges. Some meshes may contain degenerate elements, such as non-manifold mesh elements, which may be removed, to the benefit of later processing. Other mesh pre-processing operations are possible in accordance with aspects of this disclosure. 3D meshes are commonly formed using triangles, but may in other implementations be formed using quadrilaterals, pentagons, or some other n-sided polygon. In some implementations, a 3D mesh may be converted to one or more voxelized geometries (i.e., comprising voxels), such as in the case that sparse processing is performed. The techniques of this disclosure which operate on 3D meshes may receive as input one or more tooth meshes (e.g., arranged in one or more dental arches). Each of these meshes may undergo pre-processing before being input to the predictive architecture (e.g., including at least one of an encoder, decoder, pyramid encoder-decoder and U-Net). This pre-processing may include the conversion of the mesh into lists of mesh elements, such as vertices, edges, faces or in the case of sparse processing-voxels. For the chosen mesh element type or types, (e.g., vertices), feature vectors may be generated. In some examples, one feature vector is generated per vertex of the mesh. Each feature vector may contain a combination of spatial and/or structural features, as specified in the following table:

TABLE 1

| Element | Spatial Features | Structural Features |
|---|---|---|
| Edges | XYZ position of an edge | Edge curvature (depends on a |
| | midpoint, XYZ positions of | connectivity neighborhood, |
| | the edge vertices, or the | average curvature of two |
| | normal vector at an edge | vertices), dihedral angles, edge |
| | midpoint (average of the | length, density measure such as |
| | normal vectors of two | a count of incident edges (i.e., a |
| | vertices). | count of the other neighboring |
| | | edges which share the vertices |
| | | of that edge). |
| Faces | XYZ position of a face | Face curvature (average |
| | centroid, surface normal | curvature of the vertices of the |
| | vector. | face), face area, density measure |
| | | such as count of adjacent faces |
| | | (i.e., which share at least one |
| | | edge with the face). |
| Vertices | XYZ position, normal vector | Vertex curvature, density |
| | (weighted average of the | measure such as the count of |
| | normal vectors of the | vertices within a radius of the |
| | connecting faces for the | vertex, density measure such as |
| | vertex). | the count of incident edges. |
| Voxels | XYZ centroid. | Volume, [height × depth × |
| | | width] dimensions, density |
| | | measure such as a count of |
| | | contained vertices, density |
| | | measure such as count of |
| | | intersected faces, density |
| | | measure such as count of |
| | | intersected edges. |

Table 1 discloses non-limiting examples of mesh element features. In some implementations, color (or other visual cues/identifiers) may be considered as a mesh element feature in addition to the spatial or structural mesh element features described in Table 1. As used herein (e.g., in Table 1), a point differs from a vertex in that a point is part of a 3D point cloud, whereas a vertex is part of a 3D mesh and may have incident faces or edges. A dihedral angle (which may be expressed in either radians or degrees) may be computed as the angle (e.g., a signed angle) between two connected faces (e.g., two faces which are connected along an edge). A sign on a dihedral angle may reveal information about the convexity or concavity of a mesh surface. For example, a positively signed angle may, in some implementations, indicate a convex surface. Furthermore, a negatively signed angle may, in some implementations, indicate a concave surface. To calculate the principal curvature of a mesh vertex, directional curvatures may first be calculated to each adjacent vertex around the vertex. These directional curvatures may be sorted in circular order (e.g., 0, 49, 127, 210, 305 degrees) in proximity to the vertex normal vector and may comprise a subsampled version of the complete curvature tensor. Circular order means: sorted in by angle around an axis. The sorted directional curvatures may contribute to a linear system of equations amenable to a closed form solution which may estimate the two principal curvatures and directions, which may characterize the complete curvature tensor. Consistent with Table 1, a voxel may also have features which are computed as the aggregates of the other mesh elements (e.g., vertices, edges and faces) which either intersect the voxel or, in some implementations, are predominantly or fully contained within the voxel. Rotating the mesh may not change structural features but may change spatial features. And, as described elsewhere in this disclosure, the term “mesh” should be considered in a non-limiting sense to be inclusive of 3D mesh, 3D point cloud and 3D voxelized representation. In some implementations, apart from mesh element features, there are alternative methods of describing the geometry of a mesh, such as 3D keypoints and 3D descriptors. Examples of such 3D keypoints and 3D descriptors are found in “TONIONI A, et al. in ‘Learning to detect good 3D keypoints.’, Int J Comput. Vis. 2018 Vol. 126, pages 1-20.”. 3D keypoints and 3D descriptors may, in some implementations, describe extrema (either minima or maxima) of the surface of a 3D representation. In some implementations, one or more mesh element features may be computed, at least in part, via deep feature synthesis (DFS), e.g. as described in: J. M. Kanter and K. Veeramachaneni, “Deep feature synthesis: Towards automating data science endeavors,” 2015 IEEE International Conference on Data Science and Advanced Analytics (DSAA), 2015, pp. 1-10, doi: 10.1109/DSAA.2015.7344858.

Mesh element feature vectors may, in some implementations, be computed for a 3D representation which is provided to a Latent Encoding Module 214 or 218 (e.g., a representation generation neural network). For example, when a 3D mesh of a tooth is provided to a Latent Encoding Module 214 or 218, a mesh element feature vector may be computed for one or more of the 3D mesh's mesh elements, to improve the accuracy of the resulting latent representation (e.g., the latent representation generated by the Latent Encoding Module 214 or 218).

Representation generation neural networks based on autoencoders, U-Nets, transformers, 3D SWIN transformers, other types of encoder-decoder structures, convolution and/or pooling layers, or other models may benefit from the use of mesh element features. Mesh element features may convey aspects of a 3D representation's surface shape and/or structure to the neural network models of this disclosure. Each mesh element feature describes distinct information about the 3D representation that may not be redundantly present in other input data that are provided to the neural network. For example, a vertex curvature may quantify aspects of the concavity or convexity of the surface of a 3D representation which would not otherwise be understood by the network. Stated differently, mesh element features may provide a processed version of the structure and/or shape of the 3D representation, data that would not otherwise be available to the neural network. This processed information is often more accessible, or more amenable for encoding by the neural network. A system implementing the techniques disclosed herein has been utilized to run a number of experiments on 3D representations of teeth. For example, mesh element features have been provided to a representation generation neural network which is based on a U-Net model, and also to a representation generation model based on a variational autoencoder with continuous normalizing flows. Based on experiments, it was found that systems using a full complement of mesh element features (e.g., “XYZ” coordinates tuple, “Normal vector”, “Vertex Curvature”, Points-Pivoted, and Normals-Pivoted) were at least 3% more accurate than systems that did not. Points-Pivoted describes “XYZ” coordinates tuples that have local coordinate systems (e.g., at the centroid of the respective tooth). Normals-Pivoted describes “Normal Vectors” which have local coordinate systems (e.g., at the centroid of the respective tooth). Furthermore, training converges more quickly when the full complement of mesh element features are used. Stated another way, the machine learning models trained using the full complement of mesh element features tended to be more accurate more quickly (at earlier epochs) than systems which did not. For an existing system observed to have a historical accuracy rate of 91% accurate, an improvement in accuracy of 3% reduces the actual error rate by more than 30%.

Predictive models which may operate on feature vectors of the aforementioned features include but are not limited to: GDL Setups, RL Setups, VAE Setups, Capsule Setups, MLP Setups, Diffusion Setups, other Denoising Diffusion Models, PT Setups, Similarity Setups, Tooth Classification, Setups Classification, Setups Comparison, VAE Mesh Element Labeling, MAE Mesh In-filling, Mesh Reconstruction Autoencoder, Validation Using Autoencoders, Mesh Segmentation, Coordinate System Prediction, Mesh Cleanup, Restoration Design Generation, Appliance Component Generation and/or Placement, or Archform Prediction. Such feature vectors may be presented to the input of a predictive model. In some implementations, such feature vectors may be presented to one or more internal layers of a neural network which is part of one or more of those predictive models.

As described herein, tooth movements may specify one or more tooth transformations that can be encoded in various ways to specify tooth positions and orientations within the setup and are applied to 3D representations of teeth. For instance, according to particular implementations, the tooth positions can be cartesian coordinates of a tooth's canonical origin location which is defined in some semantic context. Tooth orientations can be represented as rotation matrices, unit quaternions, or other 3D rotation representations such as Euler angles with respect to a frame of reference (either global or local). Dimensions are real valued 3D spatial extents and gaps can be binary presence indicators or real valued gap sizes between teeth especially in instances when certain teeth are missing. In some implementations, tooth rotations may be described by 3×3 matrices (or by matrices of other dimensions). Tooth position and rotation information may, in some implementations, be combined into the same transform matrix, for example, as a 4×4 matrix, which may reflect homogenous coordinates. In some instances, affine spatial transformation matrices may be used to describe tooth transformations, for example, the transformations which describe the maloccluded pose of a tooth, an intermediate pose of a tooth and/or a final setup pose of a tooth. Some implementations may use relative coordinates, where setup transformations are predicted relative to malocclusion coordinate systems (e.g., a malocclusion-to-setup transformation is predicted instead of a setup coordinate system directly). Other implementations may use absolute coordinates, where setup coordinate systems are predicted directly for each tooth. In the relative mode, transforms can be computed with respect to the centroid of each tooth mesh (vs the global origin), which is termed “relative local.” Some of the advantages of using relative local coordinates include eliminating the need for malocclusion coordinate systems (landmarking data) which may not be available for all patient case datasets. Some of the advantages of using absolute coordinates include simplifying the data preprocessing as mesh data are originally represented as relative to the global origin. These details about tooth position encoding and tooth orientation encoding may, in some implementations, also apply one or more of the neural networks models of the present disclosure, including but not limited to: GDL Setups, RL Setups, VAE Setups, Capsule Setups, MLP Setups, Diffusion Setups, other Denoising Diffusion Models, PT Setups, Similarity Setups, FDG Setups, Setups Classification, Setups Comparison, VAE Mesh Element Labeling, MAE Mesh In-filling, Mesh Reconstruction VAE, and Validation Using Autoencoders.

According to particular implementations, convolution layers in the various 3D neural networks described herein may use edge data to perform mesh convolution. The use of edge information guarantees that the model is not sensitive to different input orders of 3D elements. In addition to or separate from using edge data, the convolution layers may use vertex data to perform mesh convolution. The use of vertex information is advantageous in that there are typically fewer vertices than edges or faces, so vertex-oriented processing may lead to a lower processing overhead and lower computational cost. In addition to or separate from using edge data or vertex data, the convolution layers may use face data to perform mesh convolution. Furthermore, in addition to or separate from using edge data, vertex data, or face data, the convolution layers may use voxel data to perform mesh convolution. The use of voxel information is advantageous in that, depending on the granularity chosen, there may be significantly fewer voxels to process compared to the vertices, edges or faces in the mesh. Sparse processing (with voxels) may lead to a lower processing overhead and lower computational cost (especially in terms of computer memory or RAM usage).

Representation generation neural networks based on autoencoders, U-Nets, transformers, other types of encoder-decoder structures, convolution and/or pooling layers, or other models may benefit from the use of oral care arguments (e.g., oral care metrics or oral care parameters). For example, oral care metrics (e.g., orthodontic metrics or restoration design metrics) may convey aspects of the shape and/or structure of the patient's dentition (e.g., the shape and/or structure of an individual tooth, or the special relationships between two or more teeth) to the neural network models of this disclosure. Each oral care metric describes distinct information about the patient's dentition that may not be redundantly present in other input data that are provided to the neural network. For example, an “Overbite” metric may quantify the overlap between the upper and lower central incisors along the vertical Z-axis, information which may not otherwise, in some implementations, be readily ascertainable by a traditional neural network. Stated another way, the oral care metrics provide refined information about the patient's dentition that a traditional neural network (e.g., a representation generation neural network) may not be adequately trained or configured to extract the oral care metrics described herein. However, a neural network which is specifically trained to generate oral care metrics may overcome such a shortcoming, because, for example loss may be computed in such a way as to facilitate accurate oral care metrics prediction. Mesh oral care metrics may provide a processed version of the structure and/or shape of the patient's dentition, data which may not otherwise be available to the neural network. This processed information is often more accessible, or more amenable for encoding by the neural network. A system implementing the techniques disclosed herein has been utilized to run a number of experiments on 3D representations of teeth. For example, oral care metrics have been provided to a representation generation neural network which is based on a U-Net model. Based on experiments, it was found that systems using oral care metrics (e.g., “Overbite”, “Overjet” and “Canine Class Relationship” metrics) were at least 2.5% more accurate than systems that did not. Furthermore, training converges more quickly when the oral care metrics are used. Stated another way, the machine learning models trained using oral care metrics tended to be more accurate more quickly (at earlier epochs) than systems which did not. For an existing system observed to have a historical accuracy rate of 91%, an improvement in accuracy of 2.5% reduces the actual error rate by almost 30%.

Oral care arguments may include oral care parameters, or oral care metrics. Examples of oral care metrics include Orthodontic Metrics (OM) and Restoration Design Metrics (RDM). RDM may describe the shape and/or form of one or more 3D representations of teeth for use in dental restoration. One use case example is in the creation of one or more dental restoration appliances. Another use case example is in the creation of one or more veneers (such as a zirconia veneer). Some RDM may quantify the shape and/or other characteristics of a tooth. Other RDM may quantify relationships (e.g., spatial relationships) between two or more teeth. RDM differ from restoration design parameters (RDP) in that restoration design metrics define a current state of a patient's dentition, whereas restoration design parameters serve as specifications to a machine learning or other optimization model to generate desired tooth shapes and/or forms. RDM describe the shapes of the teeth currently (e.g., in a starting or mal condition). Restoration design parameters specify how an oral care provider (such as a dentist or dental technician) intends for the teeth to look after the completion of restoration treatment. Either or both of RDM and RDP may be provided a neural network or other machine learning or optimization algorithm for the purpose of dental restoration. In some implementations, RDM may be computed on the pre-restoration dentition of the patient (i.e., the primary implementation). In other implementations, RDM may be computed on the post-restoration dentition of the patient. A restoration design may comprise one or more teeth and may be referred to as a restoration arch. Restoration design generation may involve the generation of an improved geometry and/or structure of one or more teeth in a restoration arch.

Aspects of RDM calculation are described below. In some implementations, RDM may be measured, for example, through locating landmarks in the teeth (or gums, hardware and/or other elements of the patient's dentition), and the measurements of distances between those landmarks, or otherwise made in relation to those landmarks. In some implementations, one or more neural networks or other machine learning models may be trained to identify or extract one or more RDM from one or more 3D representations of teeth (or gums, hardware and/or other elements of the patient's dentition). Techniques of this disclosure may use RDM in various ways. For instance, in some implementations, one or more neural networks or other machine learning models may be trained to classify or label one or more setups, arches, dentitions or other sets of teeth based at least in part on RDM. As such, in these examples, RDMs form a part of the training data used for training these models.

Aspects of a tooth mesh reconstruction autoencoder may be used in accordance with techniques of this disclosure are described below. An autoencoder for restoration design generation is disclosed in U.S. Provisional Application No. 63/366,514. This autoencoder (e.g., a variational autoencoder or VAE) takes as input a tooth mesh (or other 3D representation) that reflects a mal state (i.e., the pre-restoration tooth shape). The encoder component of the autoencoder encodes that tooth mesh to a latent form (e.g., a latent vector). Modifications may be applied to this latent vector (e.g., based on a mapping of the latent space through prior experiments), for the purpose of altering the geometry and/or structure of the eventual reconstructed mesh. Additional vectors may, in some implementations, be included with the latent vector (e.g., through concatenation), and the resulting concatenation of vectors may be reconstructed by way of the decoder component of the autoencoder into a reconstructed tooth mesh which is a facsimile of the input tooth mesh.

RDM and RDP may also be used as neural network inputs in the execution phase, in accordance with aspects of this disclosure. In some implementations, one or more RDM may be concatenated with the input to the encoder, for the purpose of telling the encoder specific information about the input 3D tooth representation. In some implementations, one or more RDM may be concatenated with the latent vector, before reconstruction, for the purpose of providing the decoder component with specific information about the input 3D tooth representation. Furthermore, in some implementations, one or more restoration design parameters (RDP) may be concatenated with the input to the encoder component, for the purpose of providing the encoder specific information about the input 3D tooth representation. Likewise, in some implementations, one or more restoration design parameters (RDP) may be concatenated with the latent vector, before reconstruction, for the purpose of providing the decoder specific information about the input 3D tooth representation.

In this way, either or both of RDM and RDP may be introduced to the functioning of an autoencoder (e.g., a tooth reconstruction autoencoder), and serve to influence the geometry and/or structure of the reconstructed restoration design (i.e., influence the shape of the tooth on the output of the autoencoder). In some implementations, the variational autoencoder of US Provisional Application No. U.S. 63/366,514 may be replaced by a capsule autoencoder (e.g., instead of encoding the tooth mesh to a latent vector, the tooth mesh is encoded to one or more latent capsules).

In some implementations, clustering or other unsupervised techniques may be performed on RDM to cluster one or more setups, arches, dentitions, or other sets of teeth based on the restoration characteristics of the teeth. Such clusters may be useful in treatment planning, as the clusters provide insight into categories of patients with different treatment needs. This information may be instructive to clinicians as they learn about possible treatment options. In some instances, best practices may be identified (such as default RDP values) for patient cases that fall into one or another cluster (e.g., as determined by a similarity measure, as in k-NN). After a new case is classified into a particular cluster, information about the relevant best practices may be provided to the clinician who is responsible for processing the case. Such default values may, in some instances, undergo further tuning or modifications.

Case Assignment: Such clusters may be used to gain further insight into the kinds of patient cases which exist in a dataset. Analysis of such clusters may reveal that patient treatment cases with certain RDM values (or ranges of values) may take less time to treat (or alternatively more time to treat). Cases which take more time to treat (or are otherwise more difficult) may be assigned to experienced or senior technicians for processing. Cases which take less time to treat may be assigned to newer or less-experienced techniques for processing. Such an assignment may be further aided by finding correlations between RDM values for certain cases and the known processing durations associated with those cases.

The following RDM may be measured and used in the creation of either or both of dental restoration appliances and veneers {veneers are a type of dental restoration appliance}, with the objective of making the resulting teeth natural looking. Symmetry is generally a preferred facet. There may be differences between patients based on demographic differences. The generation of dental restoration appliances may benefit from some or all of the following RDM. Shade and translucency may pertain, in particular, to the creation of veneers, though some implementations of dental restoration appliances may also consider this information.

Examples of inter-tooth RDM are enumerated below.

1) Bilateral Symmetry and/or Ratios: A measure of the symmetry between one or more teeth and one or more other teeth on opposite sides of the dental. For example, for a pair of corresponding teeth, a measure of the width of each tooth. In one instance, the one tooth is of normal width, and the other tooth is too narrow. In another instance, both teeth are of normal width. The following is a list of attributes that can be measured for a tooth, and compared to the corresponding measurement for one or more corresponding teeth: a) width-mesial to distal distance; b) length-gingival to incisal distance; c) diagonal-distance across the tooth, e.g., from the mesial gingival corner to the distal incisal corner (this measure is one of many that can be used to quantify the shape of teeth beyond length and width). Ratios between a and b may be computed, such as a/b or b/a. Such ratios can be indicative of whether spatial symmetry exists (e.g., by measuring the ratio a/b on the left side and measuring the ratio a/b on the right side, then compare the left and right ratios). In some implementations, where spatial symmetry is “off”, the length, width and/or ratios may not match. Such a ratio may, in some implementations, be computed relative to a standard. A number of esthetic standards are available in the dental literature. Examples include Golden Proportion and Recurring Esthetic Dental Proportion. In some implementations, spatial symmetry may be measured on a pair of teeth, where one tooth is on the right side of the arch, and the other tooth is on the left side of the arch.

2) Proportions of Adjacent Teeth: Measure the width proportions of adjacent teeth as measured as a projection along an arch onto a plane (e.g., a plane that is situated in front of the patient's face). The ideal proportions for use in the final restoration design can be, for example, the so-called golden proportions. The golden proportions relate adjacent teeth, such as central incisors and lateral incisors. This metric pertains to the measuring of these proportions as the proportions exist in the pre-restoration mal dentition. The ideal golden proportions are 1.6, 1, 0.6, for the central incisor, lateral incisor and cuspid, on a particular side (either left or right) for a particular arch (e.g., the upper arch). If one or more of these proportion values is off (e.g., in the case of “peg laterals”), the patient may wish for dental restoration treatment to correct the proportions.

3) Arch Discrepancies: A measure of any size discrepancies between the upper arch and lower arch, for example, pertaining to the widths of the teeth, for the purpose of dental restoration. For example, techniques of this disclosure may make adjacent tooth width proportion measurements in the upper arch and in the lower arch. In some implementations, Bolton analysis measurements may be made by measuring upper widths, lower widths, and proportions between those quantities. Arch discrepancies may be described in absolute measurements (e.g., in mm or other suitable units) or in terms of proportions or ratios, in various implementations.

4) Midline: A measure of the midline of the maxillary incisors, relative to the midline of the mandibular incisors. Techniques of this disclosure may measure the midline of the maxillary incisors, relative to the midline of the nose (if data about nose location is available).

5) Proximal Contacts: A measure of the size (area, volume, circumference, etc.) of the proximal contact between adjacent teeth. In the ideal circumstance, the teeth touch along the mesial/distal surfaces and the gums fill in gingivally to where the teeth touch. Black triangles may form if the gum tissue fails to fill the space below the proximal contact. In some instances, the size of the proximal contact may get progressively shorter for teeth located farther towards the posterior of the arch. In an ideal scenario, the proximal contact would be long enough so that there is an appropriately sized incisal embrasure and the gum tissue fills in the area below or gingival to the contact.

6) Embrasure: In some implementations, techniques of this disclosure may measure the size (area, volume, circumference, etc.) of an embrasure, the gap between teeth at either of the gingival or incisal edge. In some implementations, techniques of this disclosure may measure the symmetry between embrasures on opposite sides of the arch. An embrasure is based at least in part on the length of the length of the contact between teeth, and/or at least in part on the shape of the tooth. In some instances, the size of the embrasure may get progressively longer for teeth located farther towards the posterior of the arch.

Examples of Intra-tooth RDM are enumerated below, continuing with the numbering of other RDM listed above.

7) Length and/or Width: A measure of the length of a tooth relative to the width of that tooth. This metric may reveal, for example, that a patient has long central incisors. Width and length are defined as: a) width—mesial to distal distance; b) length—gingival to incisal distance; c) other dimensions of tooth body—the portions of tooth between the gingival region and the incisal edge. In some implementations, either or both of a length and a width may be measured for a tooth and compared to the length and/or width of one or more teeth.

8) Tooth Morphology: A measure of the primary anatomy of the tooth shape, such as line angles, buccal contours, and/or incisal angles and/or embrasures. The frequency and/or dimensions may be measured. In some implementations, the observed primary tooth shape aspects may be matched to one or more known styles. Techniques of this disclosure may measure secondary anatomy of the tooth shape, such as mamelon grooves. For instance, the frequency and/or dimensions may be measured. In some implementations, the observed secondary tooth shape aspects may be matched to one or more known styles. In some examples, techniques of this disclosure may measure tertiary anatomy of the tooth shape, such as perikymata or striations. For instance, the frequency and/or dimensions may be measured. In some implementations, the observed tertiary tooth shape aspects may be matched to one or more known styles.

9) Shade and/or Translucency: A measure of tooth shade and/or translucency. Tooth shade is often described by the Vita Classical or 3D Master shade guide. Tooth translucency is described by transmittance or a contrast ratio. Tooth shade and translucency may be evaluated (or measured) based on one or more of the following kinds of data pertaining to teeth: the incisal edge, incisal third, body and gingival third. The enamel layer translucency is general higher than the dentin or cementum layer. Shade and translucency may, in some implementations, be measured on a per-voxel (local) basis. Shade and translucency may, in some implementations, be measured on a per-area basis, such as an incisal area, tooth body area, etc. Tooth body may pertain to the portions of the tooth between the gingival region and the incisal edge.

10) Height of Contour: A measure of the contour of a tooth. When viewed from the proximal view, all teeth have a specific contour or shape, moving from the gingival aspect to the incisal. This is referred to as the facial contour of the tooth. In each tooth, there is a height of contour, where that shape is the most pronounced. This height of contour changes from the teeth in the anterior of the arch to the teeth in the posterior of the arch. In some implementations, this measurement may take the form of fitting against a template of known dimensions and/or known proportions. In some implementations, this measurement may quantify a degree of curvature along the facial tooth surface. In some implementations, measure the location along the contour of the tooth where the height of the curvature is most pronounced. This location may be measured as a distance away from the gingival margin or a distance away from the incisal edge, or a percentage along the length of the tooth.

PCT Application with Publication No. WO2020026117A1 is incorporated herein by reference in its entirety. WO2020026117A1 lists some examples of Orthodontic Metrics (OM). Further examples are disclosed herein. The orthodontic metrics may be used to quantify the physical arrangement of an arch of teeth for the purpose of orthodontic treatment (as opposed to restoration design metrics-which pertain to dentistry and describe the shape and/or form of one or more pre-restoration teeth, for the purpose of supporting dental restoration). These orthodontic metrics can measure how badly maloccluded the arch is, or conversely the metrics can measure how correctly arranged the teeth are. In some implementations, the GDL Setups model (or RL Setups, VAE Setups, Capsule Setups, MLP Setups, Diffusion Setups, PT Setups, Similarity Setups and FDG Setups) may incorporate one or more of these orthodontic metrics, or other similar or related orthodontic metrics. In some implementations, such orthodontic metrics may be incorporated into the feature vector for a mesh element, where these per-element feature vectors are provided to the setups prediction network as inputs. In some implementations, such orthodontic metrics may be directly consumed by a generator, an MLP, a transformer, or other neural network as direct inputs (such as presented in one or more input vectors of real numbers S, such as described elsewhere in this disclosure. The use of such orthodontic metrics in the training of the generator may improve the performance (i.e., correctness) of the resulting generator, resulting in predicted transforms which place teeth more nearly in the correct final setups poses than would otherwise be possible. Such orthodontic metrics may be provided to an encoder structure or by a U-Net structure (in the case of GDL Setups). Such orthodontic metrics may be consumed by an autoencoder, variational autoencoder, masked autoencoder or regularized autoencoder (in the case of the VAE Setups, VAE Mesh Element Labelling, MAE Mesh In-Filling). Such orthodontic metrics may be consumed by a neural network which generates action predictions as a part of a reinforcement learning RL Setups model. Such orthodontic metrics may be consumed by a classifier which applies a label to a setup arch (e.g., labels such as mal, staging or final setup). This description is non-limiting, as the orthodontic metrics may also be incorporated in other ways into the various techniques of this disclosure.

The various loss calculations of the present disclosure may, in some examples, incorporate one or more orthodontic metrics, with the advantage of improving the correctness of the resulting neural network. An orthodontic metric may be used to directly compare a predicted example to the corresponding ground truth example (such as is done with the metrics in the Setups Comparison description). In other examples, one or more orthodontic metrics may be taken from this section and incorporated into a loss computation. Such an orthodontic metric may be computed on the predicted example, and then the orthodontic metric would also be computed on the ground truth example. These two orthodontic metrics results would then be provided to the loss computation, with the advantage of improving the performance of the resulting neural network. In some implementations, one or more orthodontic metrics pertaining to the alignment of two or more adjacent teeth may be computed and incorporated into a loss function, for example, to train, at least in part, a setups prediction neural network. In some implementations, such an orthodontic metric may influence the network to align the mesial surface of a tooth with the distal surface of an adjacent tooth. Backpropagation is an example algorithm by which a neural network may be trained using one or more loss values.

In some implementations, one or more orthodontic metrics may be used to evaluate the predicted output of a neural network, such as a setups prediction. Such a metric(s) may enable the training algorithm to determine how close the predicted output is to an acceptable output, for example, in a quantified sense. In some implementations, this use of an orthodontic metric may enable a loss value to be computed which does not depend entirely on a comparison to a ground truth. In some implementations, such a use of an orthodontic metric may enable loss calculation and network training to proceed without the need for a comparison against a ground truth example. The advantage of such an approach is that loss may be computed based on a general principle or specification for the predicted output (such as a setup) rather than tying loss calculation to a specific ground truth example (which may have been defined by a particular doctor, clinician, or technician, whose treatment philosophy may differ from that of other technicians or doctors). In some implementations, such an orthodontic metric may be defined based on a FID (Frechet Inception Distance) score.

The following is a description of some of the orthodontic metrics which are used to quantify the state of a set of teeth in an arch for the purpose of orthodontic treatment. These orthodontic metrics indicate the degree of malocclusion that the teeth are in at a given stage of clear tray aligner treatment.

An orthodontic metric that can be computed using tensors may be especially advantageous when training one of the neural networks of the present disclosure, because tensor operations may promote efficient computations. The more efficient (and faster) the computation, the faster the rate at which training can proceed.

In some examples, an error pattern may be identified in one or more predicted outputs of an ML model (e.g., a transformation matrix for a predicted tooth setup, a labelling of mesh elements for mesh cleanup, an addition of mesh elements to a mesh for the purpose of mesh in-filling, a classification label for a setup, a classification label for a tooth mesh, etc.). One or more orthodontic metrics may be selected to become an input to the next round of ML model training, to address any pattern of errors or deficiencies which may be identified in the one or more predicted outputs.

Some OM may be defined relative to an archfrom coordinate frame, the LDE coordinate system. In some implementations, a point may be described using an LDE coordinate frame relative to an archform, where L, D and E correspond to: 1) Length along the curve of the archform, 2) Distance away from the archform, and 3) distance in the direction perpendicular to the L and D axes (which may be termed Eminence), respectively.

Various of the OM and other techniques of the present disclosure may compute collisions between 3D representations (e.g., of oral care objects, such as teeth). Such collisions may be computed as at least one of: 1) penetration distance between 3D tooth representations, 2) count of overlapping mesh elements between 3D tooth representations, and 3) volume of overlap between 3D tooth representations. In some implementations, an OM may be defined to quantify the collision of two or more 3D representations of oral care structures, such as teeth. Some optimization algorithms, such as setups prediction techniques, may seek to minimize collisions between oral care structures (such as teeth).

Between-arch orthodontic metrics are as follows.

Six (6) metrics for the comparison of two or more arches are listed below. Other suitable comparison orthodontic metrics are found elsewhere in this disclosure, such as in the section for the Setups Comparison technique.
1. Rotation geodesic distance (rotation between predicted example and ground truth setup example)
2. Translation distance (gap between predicted example and ground truth setup example)
3. Normalized translation distance
4. 3D alignment error that measures the distance between predicted mesh elements and ground truth mesh elements, in units of mm.
5. Normalized 3D alignment
6. Percent overlap (% overlap) by volume (alternatively % overlap by mesh elements) of predicted example and corresponding ground truth example

Within-arch orthodontic metrics are as follows.
Alignment—A 3D tooth orientation vector may be calculated using the tooth's mesial-distal axis. A 3D vector, which may be tangent vector to the archform at the position of the tooth may also be calculated. The XY components (i.e., which may be 2D vectors) may then be used to compare the orientation of the archform at the tooth's location to the tooth's orientation in XY space. Cosine similarity may be used to calculate the 2D orientation difference (angle) between the archform tangent and the tooth's mesial-distal axis.
Arch Symmetry—For each left-right pair of teeth (e.g., lower left lateral incisor and/or lower right lateral incisor) the absolute difference may be calculated between each tooth's X-coordinate and the global coordinate reference frame's X-axis. This delta may indicate the arch asymmetry for a given tooth pair. The result of such a calculation may be the mean X-axis delta of one or more tooth-pairs from the arch. This calculation may, in some implementations, be performed relative to the Y-axis with y-coordinates (and/or relative to the Z axis with Z-coordinates).
Archform D—axis Differences—May compute the D dimension difference (i.e., the positional difference in the facial-lingual direction) between two arch states, for one or more teeth. May, in some implementations, return a dictionary of the D-direction tooth movement for each tooth, with tooth UNS number as the key. May use the LDE coordinate system relative to an archform.
Archform (Lower) Length Ratio—May compute the ratio between the current lower arch length and the arch length as it was in the original maloccluded lower arch.
Archform (Upper) Length Ratio—May compute the ratio between the current upper arch length and the arch length as it was in the original maloccluded upper arch.
Archform Parallelism (Full arch)—For at least one local tooth coordinate system origin in the upper arch, the one or more nearest origins (e.g., tooth local coordinate system origins) in the lower arch. In some implementations, the two nearest origins may be used. May compute the straight line distance from the upper arch point to the line formed between the origins of the two teeth in the opposing (lower) arch. May return the standard deviation of the set of “point-to-line” distances mentioned above, where the set may be composed of the point-to-line distances for each tooth in the arch.
Archform Parallelism (Individual tooth)—This metric may share some computational elements with the archform_parallelism_global orthodontic metric, except that this metric may input the mean distance from a tooth origin to the line formed by the neighboring teeth in opposing arches (e.g., a tooth in the upper arch and the corresponding tooth in the lower arch). The mean distance may be computed for one or more such pairs of teeth. In some implementations, this may be computed for all pairs of teeth. Then the mean distance may be subtracted from the distance that is computed for each tooth pair. This OM may yield the deviation of a tooth from a “typical” tooth parallelism in the arch.
Buccolingual Inclination—For at least one molar or premolar, find the corresponding tooth on the opposite side of the same arch (i.e., for a tooth on the left side of the arch, find the same type of tooth on the right side and vice versa). This OM may compute an n-element list for each tooth (e.g. n may equal 2). This list may contain at least the tooth IDs of the teeth in each pair of teeth (e.g., LeftLowerFirstMolar and RightLowerFirstMolar in a list=[left_tooth_idx_1, right_tooth_idx_2]). Such an n-element vector may be computed for each molar and each premolar in the upper and lower arches. The buccal cusps may be identified on the molars and premolars on each of the left and right sides of the arch. Draw a line between the buccal cusps of the left tooth and the buccal cusps on the right tooth. Make a plane using this line and the z-axis of the arch. The lingual cusps may be projected onto the plane (i.e., at this point the angle of inclination may be determined). By performing an additional projection, the approximate vertical distance between the lingual cusps and the buccal cusps may be computed. This distance may be used as the buccolingual inclination OM.
Canine Overbite—The upper and lower canines may be identified. The first premolar for the given side of the mouth may be identified. On a given side of the arch, a distance may be computed between the upper canine and the lower canine, and also between the upper pre-molar and the lower pre-molar. The average (or median, or mode or some other statistic) may be computed for the measured distances. The z-component of this result indicates the degree of overbite. Overbite may be computed between any tooth in one arch and the corresponding tooth in the other arch.
Canine Overjet Contact—May calculate the collisions (e.g., collision distances) between pairs of canines on opposing arches.
Canine Overjet Contact KDE—May take an orthodontic metric score for the current patient case as input, and may convert that score into to a log-likelihood using a previously trained kernel density estimation (KDE) model or distribution. This operation may yield information about where in the distribution of “typical” values this patient case lies.
Canine Overjet—This OM may share some computational steps with the canine overbite OM. In some implementations, average distances may be computed. In some implementations, the distance calculation may compute the Euclidean distance of the XY components of a tooth in the upper arch and a tooth in the lower arch, to yield overjet (i.e., as opposed to computing the difference in Z-components, as may be performed for canine overbite). Overjet may be computed between any tooth in one arch and the corresponding tooth in the other arch.
Canine Class Relationship (also applies to first, second and third molars)—This OM may, in some implementations comprise two functions (e.g., written in Python).
   get_canine_landmarks ( ) Get landmarks for each tooth which may be used to compute the class relationship, and then, in some implementations, map those landmarks onto the global coordinate space so that measurements may be made between teeth.
   class_relationship_score_by_side ( ) May compute the average position of at least one landmark on at least one tooth in the lower arch, and may compute the same for the upper arch. Then may compute the vector from the upper arch landmark position to the lower arch landmark position, and finally projects this vector onto the lower arch to yield a quantification (e.g., as a scalar) of the amount of delta in “arch 1-axis” position there is. This OM may compute how far forward or behind the tooth is positioned on the 1-axis relative to the tooth or teeth of interest in the opposing arch.
Crossbite—Fossa in at least one upper molar may be located by finding the halfway point between distal and mesial marginal ridge saddles of the tooth. A lower molar cusp may lie between the marginal ridges of the corresponding upper molar. This OM may compute a vector from the upper molar fossa midpoint to the lower molar cusp. This vector may be projected onto the d-axis of the archform, yielding a lateral measure of distance from the cusp to the fossa. This distance may define the crossbite magnitude.
Edge Alignment—This OM may identify the leftmost and rightmost edges of a tooth, and may identify the same for that tooth's neighbor.
   The OM may then draw a vector from the leftmost edge of the tooth to the leftmost edge of the tooth's neighbor.
   The OM may then draw a vector from the rightmost edge of the tooth to the rightmost edge of the tooth's neighbor.
   The OM may then calculates the linear fit error between the two vectors.
   Such a calculation may involve making two vectors:
   Vec_tooth=right_tooths_leftside to left_tooths_leftside
   Vec_neighbor=right_tooths_rightside to left_tooths_leftside
   And then may involve computing the dot-product of these two vectors and subtracting the result from 1. (i.e., EdgeAlignment score=1-abs (dot (Vec_tooth, Vec_neighbor))).
   A score of 0 may indicate perfect alignment. A score of 1 may mean perpendicular alignment.
Incisor Interarch Contact KDE—May identify the deviation of the IncisorInterarchContact from the mean of a modeled distribution of such statistics across a dataset of one or more other patient cases.
Leveling—May compute a measure of leveling between a tooth and its neighbor. This OM may calculate the difference in height between two or more neighboring teeth. For molars, this OM may use the midpoint between the mesial and distal saddle ridges as the height of the molar. For non-molar teeth, this OM may use the length of the crown from gums to tip. In some implementations, the tip may be the origin of the local coordinate space of the tooth. Other implementations may place the origin in other locations. A simple subtraction between the heights of neighboring teeth may yield the leveling delta between the teeth (e.g., by comparing Z components).
Midline—May compute the position of the midline for the upper incisors and/or the lower incisors, and then may compute the distance between them.
Molar Interarch Contact KDE—May compute a molar interarch contact score (i.e., a collision depth or other type of collision), and then may identify where that score lies in a pre-defined KDE (distribution) built from representative cases.
Occlusal Contacts—For a particular tooth from the arch, this OM may identify one or more landmarks (e.g., mesial cusp, or central cusp, etc.). Get the tooth transform for that tooth. For each cusp on the current tooth, the cusp may be scored according to how well the cusp contacts the neighboring (corresponding) tooth in the opposite arch. A vector may be found from the cusp of the tooth in question to the vertical intersection point in the corresponding tooth of the opposing arch. The distance and/or direction (i.e., up or down) to the opposing arch may be computed. A list may be returned that contains the resulting signed distances, one for each cusp on the tooth in question.
Overbite—The upper and lower central incisors may be compared along the z-axis. The difference along the z-axis may be used as the overbite score.
Overjet—The upper and lower central incisors may be compared along the y-axis. The difference along the y-axis may be used as the overjet score.
Molar Interarch Contact—May calculate the contact score between molars and may use collision measurement(s) (such as collision depth).
Root Movement d—The tooth transforms for an initial state and a next state may be recieved. The archform axes at a point L along the archform may be computed. This OM may return a distance moved along the d-axis. This may be accomplished by projecting the root pivot point onto the d-axis.
Root Movement I—The tooth transforms for an initial state and a next state may be received. The archform axes at a point L along the archform may be computed. This OM may return a distance moved along the 1-axis. This may be accomplished by projecting the root pivot point onto the 1-axis.
Spacing—May compute the spacing between each tooth and its neighbor. The transforms and meshes for the arch may be received. The left and right edges of each tooth mesh may be computed. One or more points of interest may be transformed from local coordinates into the global arch coordinate frame. The spacing may be computed in a plane (e.g., the XY plane) between each tooth and its neighbor to the “left”. May return an array of one or more Euclidean distances (e.g., such as in the XY plane) which may represent the spacing between each tooth and its neighbor to the left.
Torque—May compute torque (i.e., rotation around and axis, such as the x-axis). For one or more teeth, one or more rotations may be converted from Euler angles into one or more rotation matrices. A component (such as a x-component) of the rotations may be extracted and converted back into Euler angles. This x-component may be interpreted as the torque for a tooth. A list may be returned which contains the torque for one or more teeth, and may be indexed by the UNS number of the tooth.

The neural networks of this disclosure may exploit one or more benefits of the operation of parameter tuning, whereby the inputs and parameters of a neural network are optimized to produce more data-precise results. One parameter which may be tuned is neural network learning rate (e.g., which may have values such as 0.1, 0.01, 0.001, etc.). Data augmentation schemes may also be tuned or optimized, such as schemes where “shiver” is added to the tooth meshes before being input to the neural network (i.e., small random rotations, translations and/or scaling may be applied to vary the dataset and make the neural network robust to variations in data).

A subset of the neural network model parameters available for tuning are as follows:
Learning rate (LR) decay rate (e.g., how much the LR decays during a training run)
Learning rate (LR). The floating-point value (e.g., 0.001) that is used by the optimizer.
LR schedule (e.g., cosine annealing, step, exponential)
Voxel size (for cases with sparse mesh processing operations)
Dropout % (e.g., dropout which may be performed in a linear encoder)
LR decay step size (e.g., decay every 10 or 20 or 30 epochs)
Model scaling, which may increase or decrease the count of layers and/or the count of parameters per layer.

Parameter tuning may be advantageously applied to the training of a neural network for the prediction of final setups or intermediate staging to provide data precision-oriented technical improvements. Parameter tuning may also be advantageously applied to the training of a neural network for mesh element labeling or a neural network for mesh in-filling. In some examples, parameter tuning may be advantageously applied to the training of a neural network for tooth reconstruction. In terms of classifier models of this disclosure, parameter tuning may be advantageously applied to a neural network for the classification of one or more setups (i.e., classification of one or more arrangements of teeth). The advantage of parameter tuning is to improve the data precision of the output of a predictive model or a classification model. Parameter tuning may, in some instances, provide the advantage of obtaining the last remaining few percentage points of validation accuracy out of a predictive or classification model.

Various neural network models of this disclosure may draw benefits from data augmentation. Examples include models of this which are trained on 3D meshes, such as GDL Setups, RL Setups, VAE Setups, Capsule Setups, MLP Setups, Diffusion Setups, PT Setups, Similarity Setups, FDG Setups, Setups Classification, Setups Comparison, VAE Mesh Element Labeling, MAE Mesh In-filling, Mesh Reconstruction VAE, and Validation Using Autoencoders. Data augmentation may increase the size of the training dataset of dental arches. Data augmentation can provide additional training examples by adding random rotations, translations, and/or rescaling to copies of existing dental arches. In some implementations of the techniques of this disclosure, data augmentation may be carried out by perturbing or jittering the vertices of the mesh, in a manner similar to that described in (“Equidistant and Uniform Data Augmentation for 3D Objects”, IEEE Access, Digital Object Identifier 10.1109/ACCESS.2021.3138162). The position of a vertex may be perturbed through the addition of Gaussian noise, for example with zero mean, and 0.1 standard deviation. Other mean and standard deviation values are possible in accordance with the techniques of this disclosure.

Some techniques of the present disclosure, for example the setups comparison technique, and the setups prediction techniques (e.g., such as GDL Setups, MLP Setups, VAE Setups and the like), may benefit from a processing step which may align (or register) arches of teeth (e.g., where a tooth may be represented by a 3D point cloud, or some other type of 3D representation described herein). Such a processing setup may, for example, be used to register a ground truth setup arch from a patient case with the maloccluded arch from that same case, before these mal and ground truth setup arches are used to train a setups prediction neural network model. Such a step may aid in loss calculation, because the predicted arch (e.g., an arch outputted by a generator) may be in better alignment with the ground truth setup arch, a condition which may facilitate the calculation of reconstruction loss, representation loss, L1 loss, L2 loss, MSE loss and/or other kinds of losses described herein. In some implementations, an iterative closest point (ICP) technique may be used for such registration. ICP may minimize the squared errors between corresponding entities, such as 3D representations. In some implementations, linear least squares calculations may be performed. In some implementations, non-linear least squares calculations may be performed. Various registration models may incorporate portions of the following algorithms, in whole or in part: Levenberg-Marquardt ICP, Least Square Rigid transformation, Robust Rigid transformation, random sample consensus (RANSAC) ICP, K-means based RANSAC ICP and Generalized ICP (GICP). Registration may, in some instances, help decrease the subjectivity and/or randomness that may, in some instances, occur in reference ground truth setup designs which have been designed by technicians (i.e., two technicians may produce different but valid final setups outputs for the same case) or by other optimization techniques.

In experiments, during training of a setups prediction model, the ground truth (or reference) setup was registered to the malocclusion (or maloccluded setup). The maloccluded teeth were provided to the setups prediction model, which generated final setup transforms for the maloccluded teeth. Loss was computed between the resulting predicted setup and the pre-registered ground truth setup, so that corresponding aspects of the two setups would line-up. The result was a more accurate loss calculation. This pre-registration operation resulted in a 6% improvement in absolute accuracy (e.g., as measured by ADD10 score), which amounts to a reduction in error rate of nearly 50% compared with conventional techniques.

Because generator networks of this disclosure can be implemented as one or more neural networks, the generator may contain an activation function. When executed, an activation function outputs a determination of whether or not a neuron in a neural network will fire (e.g., send output to the next layer). Some activation functions may include: binary step functions, or linear activation functions. Other activation functions impart non-linear behavior to the network, including: sigmoid/logistic activation functions, Tanh (hyperbolic tangent) functions, rectified linear units (ReLU), leaky ReLU functions, parametric ReLU functions, exponential linear units (ELU), softmax function, swish function, Gaussian error linear unit (GELU), or scaled exponential linear unit (SELU). A linear activation function may be well suited to some regression applications (among other applications), in an output layer. A sigmoid/logistic activation function may be well suited to some binary classification applications (among other applications), in an output layer. A softmax activation function may be well suited to some multiclass classification applications (among other applications), in an output layer. A sigmoid activation function may be well suited to some multilabel classification applications (among other applications), in an output layer. A ReLU activation function may be well suited in some convolutional neural network (CNN) applications (among other applications), in a hidden layer. A Tanh and/or sigmoid activation function may be well suited in some recurrent neural network (RNN) applications (among other applications), for example, in a hidden layer. There are multiple optimization algorithms which can be used in the training of the neural networks of this disclosure (such as in updating the neural network weights), including gradient descent (which determines a training gradient using first-order derivatives and is commonly used in the training of neural networks), Newton's method (which may make use of second derivatives in loss calculation to find better training directions than gradient descent, but may require calculations involving Hessian matrices), and conjugate gradient methods (which may yield faster convergence than gradient descent, but do not require the Hessian matrix calculations which may be required by Newton's method). In some implementations, additional methods may be employed to update weights, in addition to or in place of the techniques described above. These additional methods include the Levenberg-Marquardt method and/or simulated annealing. The backpropagation algorithm is used to transfer the results of loss calculation back into the network so that network weights can be adjusted, and learning can progress.

Neural networks contribute to the functioning of many of the applications of the present disclosure, including but not limited to: GDL Setups, RL Setups, VAE Setups, Capsule Setups, MLP Setups, Diffusion Setups, PT Setups, Similarity Setups, Tooth Classification, Setups Classification, Setups Comparison, VAE Mesh Element Labeling, MAE Mesh In-filling, Mesh Reconstruction Autoencoder, Validation Using Autoencoders, imputation of oral care parameters, 3D mesh segmentation (3D representation segmentation), Coordinate System Prediction, Mesh Cleanup, Restoration Design Generation, Appliance Component Generation and/or Placement, or Archform Prediction. The neural networks of the present disclosure may embody part or all of a variety of different neural network models. Examples include the U-Net architecture, multi-later perceptron (MLP), transformer, pyramid architecture, recurrent neural network (RNN), autoencoder, variational autoencoder, regularized autoencoder, conditional autoencoder, capsule network, capsule autoencoder, stacked capsule autoencoder, denoising autoencoder, sparse autoencoder, conditional autoencoder, long/short term memory (LSTM), gated recurrent unit (GRU), deep belief network (DBN), deep convolutional network (DCN), deep convolutional inverse graphics network (DCIGN), liquid state machine (LSM), extreme learning machine (ELM), echo state network (ESN), deep residual network (DRN), Kohonen network (KN), neural Turing machine (NTM), or generative adversarial network (GAN). In some implementations, an encoder structure or a decoder structure may be used. Each of these models provides one or more of its own particular advantages. For example, a particular neural networks architecture may be especially well suited to a particular ML technique. For example, autoencoders are particularly suited to the classification of 3D oral care representations, due to the ability to encode the 3D oral care representation into a form which is more easily classifiable.

In some implementations, the neural networks of this disclosure can be adapted to operate on 3D point cloud data (alternatively on 3D meshes or 3D voxelized representation). Numerous neural network implementations may be applied to the processing of 3D representations and may be applied to training predictive and/or generative models for oral care applications, including: PointNet, PointNet++, SO-Net, spherical convolutions, Monte Carlo convolutions and dynamic graph networks, PointCNN, ResNet, MeshNet, DGCNN, VoxNet, 3D-ShapeNets, Kd-Net, Point GCN, Grid-GCN, KCNet, PD-Flow, PU-Flow, MeshCNN and DSG-Net. Oral care applications include, but are not limited to: setups prediction (e.g., using VAE, RL, MLP, GDL, Capsule, Diffusion, etc. which have been trained for setups prediction), 3D representation segmentation, 3D representation coordinate system prediction, element labeling for 3D representation clean-up (VAE for Mesh Element labeling), in-filling of missing elements in 3D representation (MAE for Mesh In-Filling), dental restoration design generation, setups classification, appliance component generation and/or placement, archform prediction, imputation of oral care parameters, setups validation, or other validation applications and tooth 3D representation classification.

Some implementations of the techniques of this disclosure incorporate the use of an autoencoder. Autoencoders that can be used in accordance with aspects of this disclosure include but are not limited to: AtlasNet, FoldingNet and 3D-PointCapsNet. Some autoencoders may be implemented based on PointNet.

Representation learning may be applied to setups prediction techniques of this disclosure by training a neural network to learn a representation of the teeth, and then using another neural network to generate transforms for the teeth. Some implementations may use a VAE or a Capsule Autoencoder to generate a representation of the reconstruction characteristics of the one or more meshes related to the oral care domain (including, in some instances, information about the structures of the tooth meshes). Then that representation (either a latent vector or a latent capsule) may be used as input to a module which generates the one or more transforms for the one or more teeth. These transforms may in some implementations place the teeth into final setups poses. These transforms may in some implementations place the teeth into intermediate staging poses. In some implementations, a transform may be described by a 9×1 transformation vector (e.g., that specifies a translation vector and a quaternion). In other implementations, a transform may be described by a transformation matrix (e.g., a 4×4 affine transformation matrix).

In some implementations, systems of this disclosure may implement a principal components analysis (PCA) on an oral care mesh and use the resulting principal components as at least a portion of the representation of the oral care mesh in subsequent machine learning and/or other predictive or generative processing.

An autoencoder may be trained to generate a latent form of a 3D oral care representation. An autoencoder may contain a 3D encoder (which encodes a 3D oral care representation into a latent form), and/or a 3D decoder (which reconstructs that latent from into a facsimile of the inputted 3D oral care representation). Although this disclosure refers to 3D encoders and 3D decoders, the term 3D should be interpreted in a non-limiting fashion to encompass multi-dimensional modes of operation. For example, systems of this disclosure may train multi-dimensional encoders and/or multi-dimensional decoders.

Systems of this disclosure may implement end-to-end training. Some of the end-to-end training-based techniques of this disclosure may involve two or more neural networks, where the two or more neural networks are trained together (i.e., the weights are updated concurrently during the processing of each batch of input oral care data). End-to-end training may, in some implementations, be applied to setups prediction by concurrently training a neural network which learns a representation of the teeth, along with a neural network which generates the tooth transforms.

According to some of the transfer learning-based implementations of this disclosure, a neural network (e.g., a U-Net) may be trained on a first task (e.g., such as coordinate system prediction). The neural network trained on the first task may be executed to provide one or more of the starting neural network weights for the training of another neural network that is trained to perform a second task (e.g., setups prediction). The first network may learn the low-level neural network features of oral care meshes and be shown to work well at the first task. The second network may exhibit faster training and/or improved performance by using the first network as a starting point in training. Certain layers may be trained to encode neural network features for the oral care meshes that were in the training dataset. These layers may thereafter be fixed (or be subjected to minor changes over the course of training) and be combined with other neural network components, such as additional layers, which are trained for one or more oral care tasks (such as setups prediction). In this manner, a portion of a neural network for one or more of the techniques of the present disclosure (e.g., setups prediction) may receive initial training on another task, which may yield important learning in the trained network layers. This encoded learning may then be built upon with further task-specific training of another network.

Systems of this disclosure may train ML models with representation learning. The advantages of representation learning include the fact that the generative network (e.g., neural network that predicts a transform for use in setups prediction) can be configured to receive input with a known size and/or standard format, as opposed to receiving input with a variable size or structure. Representation learning may produce improved performance over other techniques, because noise in the input data may be reduced (e.g., because the representation generation model extracts the important aspects of an inputted representation (e.g., a mesh or point cloud) through loss calculations or network architectures chosen for that purpose). Such loss calculation methods may include KL-divergence loss, reconstruction loss or other losses disclosed herein. Representation learning may reduce the size of the dataset required for training a model, because the representation model learns the representation, enabling the generative network to focus on learning the generative task. The result may be improved model generalization because meaningful features of the input data (e.g., local and/or global features) are made available to the generative network. In some instances, transfer learning may first train a representation generation model. That representation generation model (in whole or in part) may then be used to pre-train a subsequent model, such as a generative model (e.g., that generates transform predictions). A representation generation model may benefit from taking mesh element features as input, to improve the understanding of the structure and/or shape of the inputted 3D oral care representations in the training dataset.

In accordance with this disclosure, transfer learning may be used for setups prediction, as well as for other oral care applications, such as mesh classification (e.g., tooth or setups classification), mesh element labeling, mesh element in-filling, procedure parameter imputation, mesh segmentation, coordinate system prediction, restoration design generation, mesh validation (for any of the applications disclosed herein). In some implementations, a neural network trained to output predictions based on oral care meshes may first be partially trained on one of the following publicly available datasets, before being further trained on oral care data: Google PartNet dataset, ShapeNet dataset, ShapeNetCore dataset, Princeton Shape Benchmark dataset, ModelNet dataset, ObjectNet3D dataset, Thingi10K dataset (which is especially relevant to 3D printed parts validation), ABC: A Big CAD Model Dataset For Geometric Deep Learning, ScanObjectNN, VOCASET, 3D-FUTURE, MCB: Mechanical Components Benchmark, PoseNet dataset, PointCNN dataset, MeshNet dataset, MeshCNN dataset, PointNet++dataset, PointNet dataset, or PointCNN dataset.

In some implementations, a neural network which was previously trained on a first dataset (either oral care data or other data) may subsequently receive further training on oral care data and be applied to oral care applications (such as setups prediction). Transfer learning may be employed to further train any of the following networks: GCN (Graph Convolutional Networks), PointNet, ResNet or any of the other neural networks from the published literature which are listed above.

In some implementations, a first neural network may be trained to predict coordinate systems for teeth (such as by using the techniques described in WO2022123402A1 or US Provisional Application No. U.S. 63/366,492). A second neural network may be trained for setups prediction, according to any of the setups prediction techniques of the present disclosure (or a combination of any two or more of the techniques described herein). Transfer learning may transfer at least a portion of the knowledge or capability of the first neural network to the second neural network. As such, transfer learning may provide the second neural network an accelerated training phase to reach convergence. In some implementations, the training of the second network may, after being augmented with the transferred learning, then be completed using one or more of the techniques of this disclosure.

Systems of this disclosure may train ML models with representation learning. The advantages of representation learning include that the generative network (e.g., neural network that predicts a transform for use in setups prediction) can be configured to receive input with a known size and/or standard format, as opposed to receiving input with a variable size or structure. Representation learning may produce improved performance over other techniques, because noise in the input data may be reduced (e.g., because the representation generation model extracts hierarchical neural network features and/or reconstruction characteristics of an inputted representation (e.g., a mesh or point cloud) through loss calculations or network architectures chosen for that purpose).

Reconstruction characteristics may comprise values in of a latent representation (e.g., a latent vector) that describe aspects of the shape and/or structure of the 3D representation that was provided to the representation generation module that generated the latent representation. The weights of the encoder module of a reconstruction autoencoder, for example, may be trained to encode a 3D representation (e.g., a 3D mesh, or others described herein) into a latent vector representation (e.g., a latent vector). Stated another way, the capability to encode a large set (e.g., hundreds, thousands or millions) of mesh elements into a latent vector (e.g., of hundreds or a thousand real values—e.g., 512, 1024, etc.) may be learned by the weights of the encoder. Each dimension of that latent vector may contain a real number which describes some aspect of the shape and/or structure of the original 3D representation. The weights of the decoder module of the reconstruction autoencoder may be trained to reconstruct the latent vector into a close facsimile of the original 3D representation. Stated another way, the capability to interpret the dimensions of the latent vector, and to decode the values within those dimensions, may be learned by the decoder. In summary, the encoder and decoder neural network modules are trained to perform the mapping of a 3D representation into a latent vector, which may then be mapped back (or otherwise reconstructed) into a 3D representation that is substantially similar to an original 3D representation for which the latent vector was generated.

Returning to loss calculation, examples of loss calculation may include KL-divergence loss, reconstruction loss or other losses disclosed herein. Representation learning may reduce the size of the dataset required for training a model, because the representation model learns the representation, enabling the generative network to focus on learning the generative task. The result may be improved model generalization because meaningful neural network features of the input data (e.g., local and/or global features) are made available to the generative network. Stated another way, a first network may learn the representation, and a second network may make the predictive decision. By training two networks to perform their own separate tasks, each of the networks may generate more accurate results for their respective tasks than with a single network which is trained to both learn a representation and make a decision. In some instances, transfer learning may first train a representation generation model. That representation generation model (in whole or in part) may then be used to pre-train a subsequent model, such as a generative model (e.g., that generates transform predictions). A representation generation model may benefit from taking mesh element features as input, to improve the capability of a second ML module to encode the structure and/or shape of the inputted 3D oral care representations in the training dataset.

One or more of the neural networks models of this disclosure may have attention gates integrated within. Attention gate integration provides the enhancement of enabling the associated neural network architecture to focus resources on one or more input values. In some implementations, an attention gate may be integrated with a U-Net architecture, with the advantage of enabling the U-Net to focus on certain inputs, such as input flags which correspond to teeth which are meant to be fixed (e.g,. prevented from moving) during orthodontic treatment (or which require other special handling). An attention gate may also be integrated with an encoder or with an autoencoder (such as VAE or capsule autoencoder) to improve predictive accuracy, in accordance with aspects of this disclosure. For example, attention gates can be used to configure a machine learning model to give higher weight to aspects of the data which are more likely to be relevant to correctly generated outputs. As such, and because a machine learning model configured with these attention gates (or mechanisms) utilizes aspects of the data that are more likely to be relevant to correctly generated outputs, the ultimate predictive accuracy of those machine learning models is improved.

The quality and makeup of the training dataset for a neural network can impact the performance of the neural network in its execution phase. Dataset filtering and outlier removal can be advantageously applied to the training of the neural networks for the various techniques of the present disclosure (e.g., for the prediction of final setups or intermediate staging, for mesh element labeling or a neural network for mesh in-filling, for tooth reconstruction, for 3D mesh classification, etc.), because dataset filtering and outlier removal may remove noise from the dataset. And while the mechanism for realizing an improvement is different than using attention gates, that ultimate outcome is that this approach allows for the machine learning model to focus on relevant aspects of the dataset, and may lead to improvements in accuracy similar to improvements in accuracy realized vis-à-vis attention gates.

In the case of a neural network configured to predict a final setup, a patient case may contain at least one of a set of segmented tooth meshes for that patient, a mal transform for each tooth, and/or a ground truth setup transform for each tooth. In the case of a neural network to predict a set of intermediate stage setups, a patient case may contain at least one of a set of segmented tooth meshes for that patient, a mal transform for each tooth, and/or a set of ground truth intermediate stage transforms for each tooth. In some implementations, a training dataset may exclude patient cases which contact passive stages (i.e., stages where the teeth of an arch do not move). In some implementations, the dataset may exclude cases where passive stages exist at the end of treatment. In some implementations, a dataset may exclude cases where overcrowding is present at the end of treatment (i.e., where the oral care provider, such as an orthodontist or dentist) has chosen a final setup where the tooth meshes overlap to some degree. In some implementations, the dataset may exclude cases of a certain level (or levels) of difficulty (e.g., easy, medium and hard).

In some implementations, the dataset may include cases with zero pinned teeth (or may include cases where at least one tooth is pinned). A pinned tooth may be designated by a technician as they design the treatment to stop the various tools from moving that particular tooth. In some implementations, a dataset may exclude cases without any fixed teeth (conversely, where at least one tooth is fixed). A fixed tooth may be defined as a tooth that shall not move in the course of treatment. In some implementations, a dataset may exclude cases without any pontic teeth (conversely, cases in which at least one tooth is pontic). A pontic tooth may be described as a “ghost” tooth that is represented in the digital model of the arch but is either not actually present in the patient's dentition or where there may be a small or partial tooth that may benefit from future work (such as the addition of composite material through a dental restoration appliance). The advantage of including a pontic tooth in a patient case is to leave space in the arch as a part of a plan for the movements of other teeth, in the course of orthodontic treatment. In some instances, a pontic tooth may save space in the patient's dentition for future dental or orthodontic work, such as the installation of an implant or crown, or the application of a dental restoration appliance, such as to add composite material to an existing tooth that is too small or has an undesired shape.

In some implementations, the dataset may exclude cases where the patient does not meet an age requirement (e.g., younger than 12). In some implementations, the dataset may exclude cases with interproximal reduction (IPR) beyond a certain threshold amount (e.g., more than 1.0 mm). The dataset to train a neural network to predict setups for clear tray aligners (CTA) may exclude patient cases which are not related to CTA treatment. The dataset to train a neural network to predict setups for an indirect bonding tray product may exclude cases which are not related to indirect bonding tray treatment. In some implementations, the dataset may exclude cases where only certain teeth are treated. In such implementations, a dataset may comprise of only cases where at least one of the following are treated: anterior teeth, posterior teeth, bicuspids, molars, incisors, and/or cuspids.

The mesh comparison module may compare two or more meshes, for example for the computation of a loss function or for the computation of a reconstruction error. Some implementations may involve a comparison of the volume and/or area of the two meshes. Some implementations may involve the computation of a minimum distance between corresponding vertices/faces/edges/voxels of two meshes. For a point in one mesh (vertex point, mid-point on edge, or triangle center, for example) compute the minimum distance between that point and the corresponding point in the other mesh. In the case that the other mesh has a different number of elements or there is otherwise no clear mapping between corresponding points for the two meshes, different approaches can be considered. For example, the open-source software packages CloudCompare and MeshLab each have mesh comparison tools which may play a role in the mesh comparison module for the present disclosure. In some implementations, a Hausdorff Distance may be computed to quantify the difference in shape between two meshes. The open-source software tool Metro, developed by the Visual Computing Lab, can also play a role in quantifying the difference between two meshes. The following paper describes the approach taken by Metro, which may be adapted by the neural networks applications of the present disclosure for use in mesh comparison and difference quantification: “Metro: measuring error on simplified surfaces” by P. Cignoni, C. Rocchini and R. Scopigno, Computer Graphics Forum, Blackwell Publishers, vol. 17(2), June 1998, pp 167-174.

Some techniques of this disclosure may incorporate the operation of, for one or more points on the first mesh, projecting a ray normal to the mesh surface and calculating the distance before that ray is incident upon the second mesh. The lengths of the resulting line segments may be used to quantify the distance between the meshes. According to some techniques of this disclosure, the distance may be assigned a color based on the magnitude of that distance and that color may be applied to the first mesh, by way of visualization.

Restoration treatment of the patient may involve the specification of one or more of the following: restoration guidelines, restoration design parameters, and/or restoration rules for modifying one or more aspects of a patient's dentition. One or more of many possible factors may be considered in designing a 3D restoration, whether from an esthetic standpoint and/or from a technical standpoint. For instance, from an esthetic standpoint, the dental and facial midlines and angulation may provide overall guidance, as does the amount of tooth seen by others when the lips are at rest and/or when smiling. After those criteria are considered, a set of “golden proportions” may also inform the esthetic design of overall tooth sizes. Tooth-to-tooth proportion may be configured to reflect these “golden proportions,” which are: 1.618:1.0:0.618 for the central incisor, lateral incisor and the canine, respectively. In some implementations, a real value may be specified for one or more of the RDP and be received at the input of a dental restoration design prediction model (e.g., a machine learning model to predict the final tooth shape at the completion of restoration design). In some implementations, one or more RDP may be defined which correspond to one or more restoration design metrics (RDM).

Limitations based on tooth position (i.e., malocclusion) and orientation (i.e., rotation and inclination) are balanced against trying to achieve proper symmetry, tooth proportion, and tooth-to-tooth proportion. After these parameters are established, a variety of tooth shapes may be leveraged to match the overall esthetic of the patient's face and smile. For example, tooth shapes may be generally rectangular with squared edges, or they may be generally ovoid with rounded edges. Additionally, tooth-to-tooth proportions may be manipulated to invoke a different overall esthetic. 3D Dental CAD programs often provide libraries of different tooth “styles” to choose from and offer designers the ability to tune the result to best match the esthetic and medical requirements of the doctor and patient. In some examples, symmetry may be observed in that the left side should mirror the right side, and symmetry may thereby be measured.

Tooth length, width, and width-to-length esthetic relationships may be specified for one or more teeth. In one example, the length for a maxillary central incisor may be set to 11 mm, and the width-to-length esthetic relationship may be set to either 70% or 80%. In some examples, the lateral incisors may be between 1.0 mm and 2.5 mm shorter than the central incisors. Canine teeth may, in some instances, be between 0.5 mm and 1.0 mm shorter than the central incisors. Other proportions and measurements are possible for various teeth.

From a technical standpoint, there are other considerations that may be taken into account. For instance, restorations produced from a given material must be of sufficient thickness to have the necessary mechanical strength required for long term use. Additionally, the tooth width and shape must be designed to provide a suitable contact with the adjacent teeth.

The example Style options in the list below are from the LVI standards, from the Las Vegas Institute (LVI) of Advanced Dental Studies. Other style guides are commercially or freely available.

In these and/or other examples, a neural network engine of this disclosure may incorporate as an input one or more of accepted “golden proportion” guidelines for the size of teeth, accepted “ideal” tooth shapes, patient preferences, practitioner preferences, etc.

Restoration design parameters (RDP) may be used to encode aspects of smile design guidelines described herein, such as parameters which pertain to the intended dimensions of a restored tooth. Restoration design parameters are intended as instructions and/or specifications which describe the shape and/or form that one or more teeth should assume after the completion of dental restoration treatment. One or more RDP may be received by a neural network or other machine learning or optimization algorithm for dental restoration design, with the advantage of providing guidance to that optimization algorithm. Some neural networks may be trained for dental restoration design generation, such as some examples of a GAN or an autoencoder. In some instances, a dental restoration design may be used to define the target shapes of one or more teeth for the generation of a dental restoration appliance. In some instances, a dental restoration design may be used to define the target teeth shapes for the generation of one or more veneers.

A partial list of tooth dimensions may include: length, width, height, circumference, diameter, diagonal measure, volume-any of which dimensions may be normalized in comparison to another tooth or teeth. In some implementations, one or more restoration design parameters may be defined which pertain to a gap between two or more teeth, and the amount, if any, of the gap which the patient wishes to remain after treatment (e.g., such as when a patient wishes to retain a small gap between the upper central incisors).

Additional restoration design parameters may include the parameters specified in Table 2. In the event that one of these parameters contradicts another, the following order may determine precedence (i.e., let the first parameter in the following list be considered authoritative). If a parameter value is not specified, then that parameter may be ignored. In some implementations, default values may be introduced for one or more parameters. Such default values may be determined, for example, through clustering of prior patient cases. A golden proportion guideline may specify one or more numbers pertaining to the widths of adjacent teeth, such as: {1.6, 1, 0.6}.

TABLE 2

| Parameter Name | Parameter Value of Unit of Measure |
|---|---|
| Golden proportion guideline | {guideline01, guideline02, guideline03, |
| | guideline04, etc.} |
| Tooth width at base (mesial to distal distance) | [millimeters] |
| Tooth width at incisal edge (mesial to distal | [millimeters] |
| distance) | |
| Tooth height (gingival to incisal distance) | [millimeters] |
| Width-to-length esthetic relationship | [percentage] |
| Tooth-to-tooth proportion - upper right central | [real number] |
| incisor width to upper lateral incisor width | |
| Tooth-to-tooth proportion - upper right lateral | [real number] |
| incisor width to upper cuspid width | |
| Tooth-to-tooth proportion - lower right central | [real number] |
| incisor width to lower lateral incisor width | |
| Tooth-to-tooth proportion - lower right lateral | [real number] |
| incisor width to lower cuspid width | |
| Tooth-to-tooth proportion - upper left central | [real number] |
| incisor width to upper lateral incisor width | |
| Tooth-to-tooth proportion - upper left lateral | [real number] |
| incisor width to upper cuspid width | |
| Tooth-to-tooth proportion - lower left central | [real number] |
| incisor width to lower lateral incisor width | |
| Tooth-to-tooth proportion - lower left lateral | [real number] |
| incisor width to lower cuspid width | |
| Overall tooth shape (more than one may apply) | {rectangular or ovoid, squared edges or rounded |
| | edges} |
| Amount of tooth display when the lips are at rest | [millimeters] |
| Dental restoration style | {youth, geriatric, natural, oval, etc.} |
| Style | {dominant, aggressive, focused, enhanced, |
| | functional, natural, Hollywood, mature, vigorous, |
| | youthful, oval, softened, or other examples from |
| | the LVI style guide, etc.} |
| Tooth morphology - shape style guide | [triangular, oval and rectangular, etc.] |
| Tooth morphology - angle lines | [lines_style01, lines_style02, lines_style03, etc.] |
| Tooth morphology - incisal angles and | [angles_style01, angles_style02, angles_style03, |
| embrasures | etc.] |
| Tooth morphology - buccal contour | [contour_style01, contour_style02, |
| | contour_style03, etc.] |
| Tooth morphology - Mamelon grooves | [mamelon_style01, mamelon_style02, |
| | mamelon_style03, etc.] |
| Tooth morphology - perikymata | [perikymata_style01, perikymata_style02, |
| | perikymata_style03, etc.] |

Tooth-to-tooth proportions may also be defined between other pairs of teeth as well. Proportions may be made relative to tooth widths, heights, diagonals, etc. Angle lines, incisal angles and buccal contours may describe primary aspects of tooth macro shape. Mamelon grooves may be vertical macro textures on the front of a tooth, and sometimes may take a V-shape. Striations or perikymata may be a horizontal micro texture on the teeth. Symmetry may be generally desired. There may be differences between male and female patients.

Parameters may be defined to encode doctor restoration design preferences (DRDP), as pertains to various use case scenarios. These use case scenarios, may reflect information about the treatment preferences of one or more doctors, and directly affect the characteristics of one or more teeth in a dental restoration design or a veneer. In addition, DRDP may describe preferred or habitually involved values or ranges of values of RDP for a doctor or other treating medical professional. In some instances, such value or ranges of values may be derived from historical patient cases that were treated by that doctor or medical professional. In some instances, a DRDP may be defined which is derived from a RDP (e.g., such as Width-to-length esthetic relationship) or from a RDM.

Machine learning models, such as those described herein (e.g., denoising diffusion models), may be trained to generated designs for tooth crowns or tooth roots (or both). A dental restoration design may describe a tooth shape which is intended at the end of dental restoration. A neural network (such as a generative neural network) may be trained to generate a dental restoration design which is to be used to produce either a veneer (e.g., a zirconia veneer) or a dental restoration appliance. Such a model make take as input data from cohort patient cases, including pre-restoration tooth meshes and corresponding ground truth examples of completed restorations (e.g., tooth meshes with restored shapes and/or structures). Such a model may be trained, at least in part through the calculation of a loss function, which may quantify the difference between a generated crown restoration design and a ground truth crown restoration design. The resulting loss may be used to update the weights of the generative neural network model (e.g., a denoising diffusion model—which may include a U-Net), thereby training the model (at least in part). A reconstruction loss may be computed to compare a predicted tooth mesh to a ground truth tooth mesh, or to compare pre-restoration tooth mesh to a completed restoration design tooth mesh. Reconstruction loss may be computed as the sum of pairwise distances between corresponding mesh elements and may be computed to quantify the difference between two tooth crown designs. Other losses disclosed herein may also be in the training. A veneer may be created using the generated restoration design. Such as veneer may be 3D printed.

Machine learning models, such as those described herein (e.g., denoising diffusion models), may be trained to generate components for use in creating a dental restoration appliance. Such a dental restoration appliance may be used to shape dental composite in the patient's mouth while that composite is cured (e.g., using a curing light), to ultimately produce veneers on one or more of the patient's teeth. The 3M FILTEK Matrix is an example of such a product. In some instances, a machine learning model for generating an appliance component may take inputs which are operable to customize the shape and/or structure of the appliance component, including inputs such as oral care parameters. One or more oral care parameters may, in some instances, be defined based on oral care metrics. An oral care metric (e.g., an orthodontic metric or a restoration design metric) may describe physical and/or spatial relationships between two or more teeth or may describe physical and/or dimensional characteristics of an individual tooth. An oral care parameter may be defined which is intended to provide a machine learning model with guidance on generating a 3D oral care representation with a particular physical characteristic (e.g., pertaining to shape and/or structure). For example, physical characteristics may be measured with an oral care metric to which that oral care parameter corresponds. Such oral care parameters may be defined to customize the generation of mold parting surfaces, gingival trim meshes or other generated appliance components, to tailor those appliance components to the patient's dental anatomy.

In some implementations, the 3D representation generation techniques described herein (e.g., denoising diffusion-based techniques) may be trained to generate the custom appliance components by determining the characteristics of the custom appliance components, such as a size, shape, position, and/or orientation of the custom appliance components. Examples of custom appliance components include a mold parting surface, a gingival trim surface, a shell, a facial ribbon, a lingual shelf (also referred to as a “stiffening rib”), a door, a window, an incisal ridge, a case frame sparing, or a diastema matrix wrapping, a spline, among others. A spline refers to a curve that passes through a plurality of points or vertices, such as a piecewise polynomial parametric curve. A mold parting surface refers to a 3D mesh that bisects two sides of one or more teeth (e.g., separates the facial side of one or more teeth from the lingual side of the one or more teeth). A gingival trim surface refers to a 3D mesh that trims an encompassing shell along the gingival margin. A shell refers to a body of nominal thickness. In some examples, an inner surface of the shell matches the surface of the dental arch and an outer surface of the shell is a nominal offset of the inner surface. The facial ribbon refers to a stiffening rib of nominal thickness that is offset facially from the shell. A window refers to an aperture that provides access to the tooth surface so that dental composite can be placed on the tooth. A door refers to a structure that covers the window. An incisal ridge provides reinforcement at the incisal edge of dental appliance and may be derived from the archform. The case frame sparing refers to connective material that couples parts of a dental appliance (e.g., the lingual portion of a dental appliance, the facial portion of a dental appliance, and subcomponents thereof) to the manufacturing case frame. In this way, the case frame sparing may tie the parts of a dental appliance to the case frame during manufacturing, protect the various parts from damage or loss, and/or reduce the risk of mixing-up parts. These appliance components and others are described in PCT patent applications WO2020240351A1 and WO2021240290A1, both of which are incorporated herein by reference in their entirety.

A mold parting surface refers to a 3D mesh that bisects two sides of one or more teeth (e.g., by separating the facial side of one or more teeth from the lingual side of the one or more teeth). A gingival trim surface refers to a 3D mesh that trims an encompassing shell along the gingival margin. A shell refers to a body of nominal thickness. In some examples, an inner surface of the shell matches the surface of the dental arch and an outer surface of the shell is a nominal offset of the inner surface.

The facial ribbon refers to a stiffening rib of nominal thickness that is offset facially from the shell. A window refers to an aperture that provides access to the tooth surface so that dental composite can be placed on the tooth. A door refers to a structure that covers the window. An incisal ridge provides reinforcement at the incisal edge of a dental restoration appliance and may be derived from the archform. The case frame sparing refers to connective material that couples parts of a dental restoration appliance (e.g., the lingual portion of a dental restoration appliance, the facial portion of a dental restoration appliance, and subcomponents thereof) to the manufacturing case frame. In this way, the case frame sparing may tie the parts of a dental restoration appliance to the case frame during manufacturing, protect the various parts from damage or loss, and/or reduce the risk of mixing up parts.

Additional 3D oral care representations which may be generated by a denoising diffusion model (such as denoising diffusion models trained as described herein) including tooth restoration designs (e.g., which may include tooth interproximal surfaces, fossae, incisal edges, cusp tips, tooth roots or the like).

In some implementations, the denoising diffusion models (DDM) described herein may be trained to perform 3D mesh element labeling (e.g., labeling vertices, edges, faces, voxels or points) in 3D oral care representations. Those labeled mesh elements may be used for mesh cleanup or mesh segmentation. In the case of mesh cleanup, the labeled aspects of a scanned tooth mesh may be used for appliance erasure (removal+replacement) or be used to modify (e.g., by smoothing) one or more aspects of the tooth to remove aspects of attached hardware (or other aspects of the mesh which may be unwanted for certain processing and appliance creation-such as extraneous material). Mesh element features, such as those described herein, may be computed for one or more mesh elements in a 3D representation of oral care data (e.g., 3D representation of the patient's dentition). A vector of such mesh element features may be computed for each mesh element and then be received by a DDM which has been trained to label mesh elements in a 3D oral care representation for the purpose of either mesh segmentation or mesh cleanup. Such mesh element features may confer valuable information about the shape and/or structure of the input mesh to the labeling DDM. For example, training data 200 may contain 3D representations of the patient's pre-restoration dentition (e.g., from an intraoral scanner, CT scanner, or the like), and ground truth data which includes ground truth mesh element labels. The ground truth mesh element labels may describe ground truth (or reference) segmentations of the patient's dentition (e.g., each mesh element which occupies a lower left central incisor may have the same label, each mesh element in the gums of the upper arch may have the same label, each mesh element which appears in an upper right cuspid may have the same label, etc.). Either or both of the patient's dentition and the ground truth mesh element labels may undergo latent encoding (214). Either the original training data 200 or the corresponding latent-encoded training data may be provided to the Markov chain 204, as a part of the forward pass 206. The forward pass may iteratively add noise to the mesh element labels, generating a series of tens, hundreds, or thousands of successively noisier versions of the set of ground truth mesh element labels. In some implementations, the successive addition of noise may comprise randomizing the value of one or more mesh element labels. In some implementations, the successive addition of noise may comprise adjusting the labels of the neighbors of a randomly chosen mesh element (e.g., such as a mesh element which lies along the boundary of a tooth). In this manner, the boundaries between teeth, or between a tooth and the gums man be adjusted to become increasingly noisy or distorted. The Markov chain may generate a succession of increasingly noisy sets of mesh element labels which may be used to train, at least in part, the denoising ML model 210. In some implementations, the 3D representation(s) of the patient's detention may also be provided, along with the mesh element labels, to the training of the denoising ML model 210. In deployment, the reverse pass 208 may be executed. Oral care arguments 202 may be provided, to customize the functioning of the segmentation operation. In deployment, instant patient case data 216 (e.g., a pre-segmentation 3D representation of the patient's dentition) may be provided to the denoising ML model 210. In some implementations, the instant patient case data 216 may undergo latent encoding (218). In some implementations, an initial set of mesh element labels may be generated which correspond to the 3D representation of the patient's detention 216. The mesh element labels may, in some implementations, be initialized at random, or according to heuristics. An example of a heuristic is that a set of random mesh elements are chosen, and the neighbors of those mesh elements (e.g., neighbors with a particular distance or number of edges of the chosen mesh element) may be grouped together in cliques (e.g., which each mesh element in a clique has the same mesh element label). The reverse pass 208 of the denoising ML model 210 may cause the denoising ML model 210 to iteratively refine the set of mesh element labels, relative to the 3D representation of the patient's dentition 216. After many iterations (e.g., hundreds, thousands or millions) the iteratively refined mesh element labels may be set to the output 220. These generated mesh element labels may be used to segment the patient's detention or to perform mesh cleanup of the patient's dentition. In some examples, when the instant patient case data contains the patient's pre-segmentation dentition, the patient's dentition may become segmented through the application of the generated mesh element labels 220. The generated mesh element labels may enable tooth cutting (e.g., the generation of a new mesh for each distinct tooth), according to the generated mesh element labels 220.

Some implementations of the DDM-based mesh cleanup techniques described herein may train the DDMs to remove (or modify) generic triangle mesh defects (e.g., via mesh element labeling), such as: degenerate triangle with zero surface area; redundant triangle that covers the same surface area as another triangle; non-manifold edge with more than two adjacent triangles, also referred to as a “fin”; non-manifold vertex with more than one adjacent sequence of connected triangles (triangle fans); intersecting triangles—where two triangles penetrate each other; spikes-sharp features composed of multiple triangles, often conical, caused by one or more vertices being displaced from the actual surface; folds-sharp features composed of multiple triangles, often Z-shaped with a small undercut area, caused by one or more vertices being displaced from the actual surface; islands/small components—disconnected objects in a scan which should only contain a single object (e.g., typically the smaller objects are deleted); small holes in the mesh surface, either from the original scan or from deletions due to the previous defects (e.g., the hole may be removed by filling the hold, for example by adding one or more mesh elements); rough boundary-a smooth boundary is desirable for extending the gingiva surface, creating a model base.

Some implementations of the DDM-based mesh cleanup techniques described herein may train the DDM models to remove (or modify) aspects of meshes (e.g., via mesh element labeling) which are unwanted under certain circumstances and/or domain-specific defects, such as: extraneous material—portions of the intraoral scan outside the anatomical area of interest, e.g., non-tooth surfaces that are not within some distance of tooth surfaces, or scan artifacts that do not represent actual anatomy; divots-concave depressions in surfaces (e.g., may be scan artifacts, which should be fixed, or anatomical features, which are generally left intact); undercuts—sides of a tooth of lower radius than the crown, such that physical impressions or aligners may become difficult to remove or emplace. Undercuts may be a natural feature or due to damage such as an abfraction. Abfractions are associated with the erosion of a tooth near the gumline, causing or exacerbating an undercut. Appliances that the DDM-based models of this disclosure may process include orthodontic hardware such as attachments, brackets, wires, buttons, lingual bars, Carriere appliances, or the like, and may be present in intraoral scans. Digital removal and replacement with synthetic tooth/gingiva surfaces may, in some circumstances, be beneficial if performed before appliance creation steps proceed.

Denoising diffusion may train a neural network (or other machine learning model) to iteratively remove noise from (or refine) a data structure. Techniques of this disclosure remove noise from a 3D point cloud (or other 3D representation), from a transformation matrix, a vector of labels (e.g., labels which are applied to mesh elements and are used for segmentation or mesh cleanup, and which may be used to define object masks) or from other kinds of 3D oral care representations. The techniques may start out with randomly generated data structures (e.g., 3D point clouds or transformation matrices), and the techniques may transform those random (or noisy) data structures into artifacts which are usable in digital oral care. Denoising models may be trained to perform these operations because of the kind of training data that is generated using the techniques described herein. This training data may comprise a series of increasingly noisy examples of the relevant data structure. The denoising neural network (of which a U-Net is an example) may be trained to gradually, in small steps, transform a random (or noisy) data structure into an artifact which is useable in digital oral care treatment (e.g., the generation of oral care appliances).

Digital oral care involves many different kinds of 3D representations which are customized to the anatomy of the patient. Diffusion models are particularly applicable to digital oral care because of the customizable nature of the generated output representations.

In some implementations, a denoising diffusion model may operate on input data (e.g., 3D oral care representations) in the data's original format (e.g., training on and/or generating 3D representations-such as 3D point clouds, and the like). In other implementations, a denoising diffusion model may operate on a latent form of the input data (e.g., such as in Stable Diffusion), which may preserve the 3D nature of the input data. A latent form may be information-rich (describing the structure and/or shape of the input data-such as a 3D oral care representation) and/or have a small data footprint that is easier for an ML model to be trained on. In some instances, an autoencoder may be trained to generate a latent representation. The information-rich nature of the latent representation may be demonstrated when the latent representation is reconstructed into a close facsimile of the original 3D representation (e.g., as measured by reconstruction error defined herein). Stated another way, the original 3D representation may comprise hundreds or thousands of mesh elements, which may be encoded by the autoencoder into a a vector of a few hundred real values, in some instances. This vector of a few hundred real values may, in some instances, subsequently be reconstructed into a close facsimile of the original 3D representation. The accuracy of the reconstruction (e.g., the fidelity with which the reconstructed form matches the original form) may be verified by computing a reconstruction error.

An oral care diffusion model (OCDM) may, in some implementations, be trained to modify the 3D oral care representations described herein (e.g., an appliance component, a tooth restoration design, a trimline, a transform for a 3D oral care representations-such as a tooth in an orthodontic setup or an appliance component, etc.). In some instances, a template or reference 3D oral care representation may be received at the input and subsequently modified by the OCDM. In some instances, an initial tooth representation (e.g., pre-restoration tooth) may be received at the input and subsequently be modified by the OCDM to generate a tooth which is ready for restoration treatment (e.g., for using making a crown, bridge, or a dental restoration appliance such as the FILTEK Matrix). An OCDM may, in some implementations, be trained to generate the 3D oral care representations described herein (e.g., an appliance component, a tooth restoration design, a trimline, an orthodontic setup, etc.). In some implementations, a diffusion model may be trained to generate a 3D oral care representation (e.g., a point cloud or 3D mesh representing a 3D oral care representation such as a trimline, an appliance component or a tooth design for dental restoration-such as the generation of a veneer, the generation of a dental restoration appliance). Such a model may be termed a Geometry Generation Diffusion neural network or Geometry Generation Diffusion Model (GGDM).

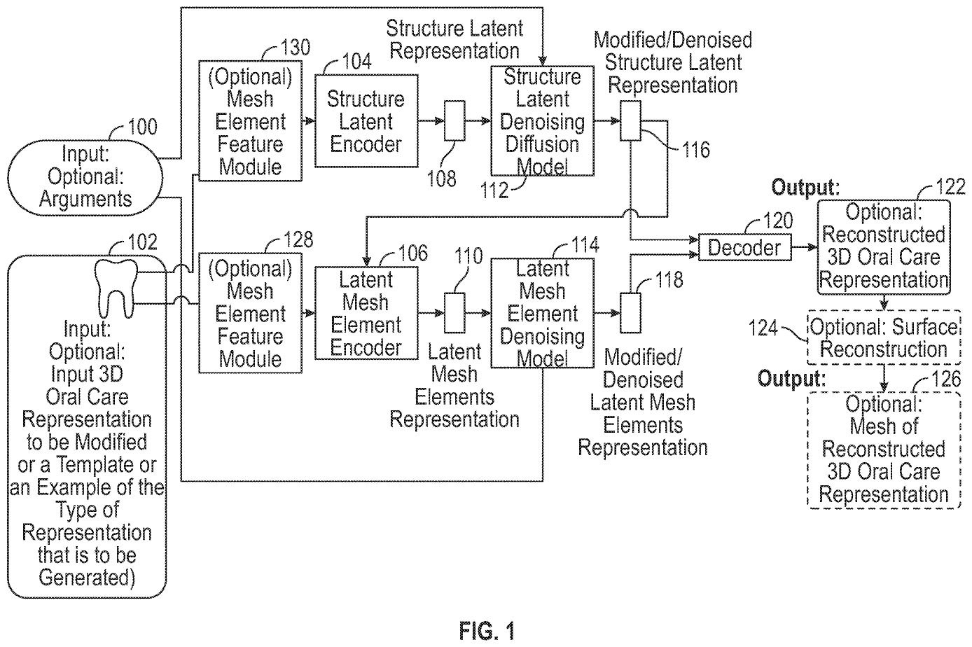

In some instances, the input data for a diffusion model (e.g., a Stable Diffusion model) may be pre-processed using an encoder (e.g., to generate a reduced dimensionality representation of the input data structure). Such a latent representation may, in some instances, be reconstructed using a decoder. The decoder may reconstruct the latent representation into a facsimile of the input data (e.g., such as with a reconstruction autoencoder). In some instances, the decoder may reconstruct the latent representation into a modified version of the Input data (e.g., when one or more aspects of the latent representation are modified). Point-Voxel CNNs (PVCNN) or encoder-decoder networks (e.g., such as Variational Autoencoders, among others) may be trained to implement the encoders or decoders of the techniques described herein. A 3D oral care representation 102 may be received at the input to the Oral Care Diffusion Model (OCDM) and be encoded to a latent representation (e.g., a latent vector) by an encoder. In some implementations, such as in FIG. 1, an optional encoder 104 (e.g., as a structure latent encoder-which may encoder aspects of the shape and/or structure of the input data) may encode an input 3D oral care representation 102 into a latent representation 108 of that shape (e.g., termed a structure latent representation). In some implementations, such as that in FIG. 1, an optional latent mesh element encoder 106 may encode an input 3D oral care representation 102 into a latent representation 110 of mesh elements (e.g., termed a latent mesh element representation). Such mesh elements may comprise points in a point cloud, voxels in a sparse representation, or vertices/edges/faces in a 3D mesh, among others. In some implementations, each of these two representations may be generated. Such encoders may, in some implementations, incorporate mesh element features at the input, to improve the latent representations generated by the encoders. Mesh element feature vectors may be computed by mesh element feature modules 130 and 128.

In the forward process, a Markov chain of diffusion steps may be applied to generate a set of training data from such a latent representation. The forward process may generate increasingly noisy versions of the input 3D oral care representation 102 which may be used in training the one or more denoising diffusion neural network models described herein. This Markov chain of diffusion steps may apply Gaussian noise to the input 3D oral care representation 102 over T steps (e.g., T==150), which may produce a succession of increasingly noisy versions of the input 3D oral care representation 102 which may now be used to train a denoising diffusion neural network. A diffusion neural network (e.g., which may be implemented with a U-Net, variational autoencoder, etc.) may be trained to reverse the forward process, starting with the 3D oral care representation at time T, and performing a denoising operation to convert the 3D oral care representation to a version which corresponds to time T−1. This is called the reverse process. Given the latent representation at time t, the diffusion neural network may compute the version of the latent representation at time t−1, in an iterative fashion. The diffusion neural network may introduce changes to the latent representation (e.g., to remove noise or to introduce geometrical or structural aspects which are desired in the output). The diffusion model may consume instructions on such changes in the form of oral care arguments. Oral care arguments may comprise of one or more natural language text string arguments, one or more categorical arguments, one or more integer arguments, one or more real-valued arguments, one or more image arguments (e.g., a reference image describing aspects of an intended outcome of model execution), one or more 3D representation arguments (e.g., a reference 3D point cloud describing aspects of an intended out of model execution) or a combination of multiple of those previously mentioned. Such arguments may first undergo latent encoding (e.g., using the encoder portion of an autoencoder or using a text or numerical encoder). The encoded arguments may be introduced to the diffusion neural network (e.g., by appending the arguments to the input data, such as concatenating the arguments with a latent representation of the input data that was generated using an encoder). For example, when the diffusion neural network is implemented using a U-Net, the encoded arguments may be introduced to one or more of the successive resolution layers of the U-Net (e.g., the U-Net may have sets of layers which correspond to increasingly lower levels of resolution, followed by sets of layers which correspond to increasingly higher levels of resolution). The U-Net may employ skip-connections to share information from input to output at each level of resolution. The U-Net may incorporate attention layers. The encoded argument information may be introduced to any or all of such layers, with the advantage of guiding the generated output of the diffusion neural network. In some implementations, the denoising diffusion neural networks of this disclosure may include U-Nets, ResNets, transformers or autoencoders (e.g., variational autoencoders), among others.

The latent representation of the shape of a 3D point cloud 102 (or other 3D representation described herein) is called a structure latent representation 108, which may encode aspects of the structure of the input 3D point cloud 102. Oral care arguments 100 (or input parameters) may comprise one or more attributes describing an intended output from a trained machine learning model. A structure latent representation 108 may undergo denoising and/or modification by a structure latent denoising diffusion model 112. The latent representation of mesh elements (e.g., such as points) is called a latent mesh elements representation 110 (e.g., which may encode aspects of the shape of the input 3D point cloud 102). A latent mesh elements representation may undergo denoising and/or modification by a latent mesh element denoising diffusion model 114. The denoised/modified structure latent representations 116 may be provided to a final decoder module 120 (e.g., implemented as a PVCNN). The latent mesh elements representation 118 may also be provided to the final decoder module 120. The final decoder module may reconstruct a generated geometry 122 (e.g., or other 3D oral care representation) which is either a modified version of the input 3D oral care representation 102 or is an entirely new 3D oral care representation with aspects as specified by the oral care arguments 100. In the case that the input 3D oral care representation is embodied by 3D point cloud points, the generated 3D oral care representation 122 may be embodied by 3D point cloud points. Such a point cloud may undergo an optional subsequent processing step 124 to produce a mesh, for example, using Shape As Points (SAP) surface reconstruction (e.g., as shown in FIG. 1). If SAP is applied, then a 3D mesh 126 is outputted. In other instances, the input 102 and reconstructed 122 3D oral care representations may be embodied as voxels, or other of the mesh elements described herein.

FIG. 1 shows a method of using a fully trained GGDM. The GGDM has been trained to modify an input 3D oral care representation according to (optional) arguments (e.g., as text instructions or according to oral care parameters described herein).

In some instances, a diffusion model or a Text-to-Image (TTI) system (e.g., such as DALL-E 2, MidJourney or Stable Diffusion) which has been initially trained to generate 2D representations (such as 2D images) may be further trained on 3D oral care representations using transfer learning. For example, denoising ML model 210 may be first trained on 2D data, and subsequently be further trained (via transfer learning) on 3D data. Such a transfer-learned model may, in some implementations, be trained to generate 3D oral care representations. In some instances, a stable diffusion model may operate on latent representations of trial 3D oral care representations 216, for the purpose of modifying those 3D oral care representations 216.

In some non-limiting implementations, the denoising diffusion probabilistic models of this disclosure may operate on 3D representations of teeth (e.g., to generate tooth restoration designs). In the example of restoration design generation, a 3D representation of the pre-restoration tooth 102 (e.g., described by as a 3D point cloud, 3D mesh or voxelized representation) may be provided as the input, and processed by denoising diffusion probabilistic neural networks in a manner that preserves information about the 3D shape and/or 3D structure of that pre-restoration tooth. In some instances, the pre-restoration tooth may undergo optional mesh element feature vector generation (128), and optionally undergo encoding into latent form (106) which preserves information about the 3D qualities of the pre-restoration tooth. The resulting latent mesh elements representation 110 may represent the 3D mesh elements (e.g., 3D points, voxel, edges, faces, or vertices) of the pre-restoration tooth in latent form. The latent mesh elements representations 110 may be provided to the latent mesh element denoising diffusion model 114, which may generate a modified latent mesh elements representation 118. In some implementations, the pre-restoration tooth may undergo optional mesh element vector generation (130), and optionally undergo encoding into a latent form (104) which describes aspects of the pre-restoration tooth's 3D structure in latent form 108. The latent representation 108 may be provided to structure latent denoising diffusion model 112, which may generate a modified structure latent representation 116. Either or both of the modified structure latent representation 116 and the modified latent mesh elements representation 118 may be provided to the decoder 120, which may reconstruct the representations into a reconstructed 3D oral care representation 122 (e.g., a post-restoration tooth design). When the reconstructed 3D oral care representation comprises a point cloud, that point cloud may subsequently undergo surface reconstruction (124), to generate a 3D mesh 126. Oral care arguments 100 may optionally be provided to either or both of the structure latent denoising diffusion model 112, and the latent mesh elements denoising diffusion model 114, to customize the outputs of those models, and configure those models to generate output which is suitable for use in generation an oral care appliance. Oral care arguments may contain oral care parameters or oral care metrics (e.g., restoration design metrics-such as “Tooth Morphology,” or “Bilateral Symmetry and/or Ratios,” among others described herein).

Such latent forms 110 and 108 may reduce the data size of the pre-restoration tooth, leading to improvements in data precision, while still preserving rich information about the shape and/or structure of the pre-restoration tooth. Neural networks may be more easily trained (e.g., require fewer neural network parameters or weights to encode solutions) on input data with a smaller data footprint, as long as those data are information-rich. Reconstruction autoencoders are particularly configured to generate information-rich latent representations, as shown by the reconstruction autoencoder's ability to reconstruct a close facsimile of the input data. A ground truth post-restoration 3D representation of a tooth may likewise be processed entirely in three dimensions. A Geometry Generation Diffusion Model (GGDM) may be trained, at least in part, by computing a loss which quantifies the difference between a generated post-restoration tooth design (e.g., a design predicted using a denoising diffusion model as described herein) and a ground truth post-restoration tooth (e.g., which may be provided in the training data for a given patient case). Such a loss calculation may be performed entirely in 3D using, for example, a reconstruction loss calculation, as described herein. Techniques described herein may generate tooth designs for crowns, veneers, bridges, or for other types of restorations, or for tooth designs for use in designing dental restoration appliances, or fixture models, and the like.

A denoising diffusion model (DDM) (e.g., such as that shown in FIG. 2) may involve one or more Markov chains. A Markov chain may, in some instances, be defined as a sequence of stochastic events where each time point depends on the previous time point. The transition distributions on the Markov chain forward process may, in some instances, be conditioned by a low level of Gaussian noise. FIG. 2 shows a denoising diffusion model which is trained on structure latent data. The forward pass 206 of FIG. 2 generates training data for a denoising diffusion model (e.g., which may, in some implementations, be trained on latent representations of mesh elements). The training data may be used to train the denoising ML model 210. The fully trained denoising ML model 210 shown in FIG. 2 may be used by GGDM (e.g., where the model is trained on latent representations of teeth—such as with restoration design generation), by a Diffusion Setups model (e.g., where the model is trained on latent representations of transforms for teeth or other types of 3D oral care representations), or by other types of diffusion models for generating outputs based on training datasets of 3D oral care representations.

A DDM may be trained to approximate a data distribution q (x) over a latent variable Y (e.g., where the data distribution may describe 3D oral care representations). The forward and backward processes of the denoising diffusion model may operate over many time points t, for example T==1000. Any of the following three operations may be involved a denoising diffusion model: a noise scheduler which is used to implement the forward process (e.g., which may introduce progressively more noise of a particular distribution, such as Gaussian noise, to each successive time point—such as isotropic Gaussian noise with mean of zero and variance in all directions), a neural network (e.g., U-Net, VAE or others described herein) which is used to implement the reverse process (e.g., which may, over many time points, recover the original distribution of the data that supplied to the start of the forward process), and/or an optional method of time point encoding (e.g., using positional embeddings). The neural network may be trained, at least in part, using a loss function which is based on an L1 or L2 norm, or a KL-Divergence (among others described herein). KL-Divergence loss may, in some instances, drive minimization of the difference between an observed distribution and a corresponding generated distribution.

The forward process may, in some instances, comprise a further encoding of the received data (e.g., latent data), and the backward (or reverse) process may, in some instances, comprise a decoding (or partial decoding) of the data which were processed by the forward process. In some instances, the backward process may be performed starting with random noise, and a sequence of progressively less noisy data structures may be generated.

At the last time point, t==T, the distribution of latent variables Y may, in some instances, be normally distributed, after the addition of noise (e.g., Gaussian noise) using a forward transition module. The forward process may, in some instances, introduce noise to a 3D oral care representation by, in the example of a 3D representation of oral care data, perturbing one or more mesh elements of the 3D oral care representation. For example, the position of a point cloud point or of a voxel may be perturbed. A mesh element may undergo perturbation which may alter the value of a measured mesh element feature (e.g., length of an edge, area of a face, position of a point or voxel, count of incident edges for a vertex, etc.). The transition module of the forward process may, in some implementations, be described as (where Mₜ is a variance distribution such that q(yₜ)~=p(yₜ) and I is the identity matrix):

q(y|yₜ₋₁)=N(yₜ;sqrt(1−Mₜ)yₜ₋₁,MₜI)

The reverse pass in FIG. 2 may, in some implementations, be implemented using a neural network, such as a U-Net. The transition module for this reverse pass may, in some instances, be described by the following product from times t==1 to t==T:

p(y₀:T)=p(y_{T})πp_{w}(yₜ₋₁|yₜ), where p_{w} is Gaussian.

A U-Net may have a descending phase (in which the resolution of the input data is successively decreased using convolutional and down-sampling pooling layers, up to a level of most-coarse resolution, which may reveal increasingly global features of the input data) and an ascending phase (which receives the result of the descending phase and restores the resolution over a succession of complementary stages of unconvolution and unpooling). Skip (or residual) connections may connect corresponding stages of the descending and ascending phases. Attention modules may be integrated into the U-Net. Batch normalization layers may be integrated into the U-Net.

FIG. 2 shows an example DDM, for setups prediction. An example of the data which is processed (over time points t) by a denoising diffusion model for 3D oral care representations (e.g., for denoising latent representations of 3D oral care representations, such as structure latents or latent mesh elements). structure latents or latent mesh elements may be described by one or more latent vectors.

The denoising diffusion neural network (e.g., a U-Net) may generate a target 3D oral care representation (e.g., an appliance component, a tooth restoration design, a fixture model component-such as a trimline, etc.) by iteratively denoising a set of mesh elements (e.g., points of a 3D point cloud) until a 3D oral care representation is generated which meets the specification of the oral care arguments which are supplied to the model. Oral care arguments may include oral care parameters as disclosed herein, or other real-valued, text-based or categorical inputs which specify intended aspects of the one or more target 3D oral care representations which are to be generated. In some instances, oral care arguments may include oral care metrics, which may describe intended aspects of the one or more 3D oral care representations which are to be generated. In some instances, a text encoder may encode a set of natural language instructions from the clinician (e.g., generate a text embedding). A text string may comprise tokens. An encoder for generating text embeddings may, in some implementations, apply either mean-pooling or max-pooling between the token vectors. In some instances, a transformer (e.g., BERT or Siamese BERT) may be trained to extract embeddings of text for use in digital oral care (e.g., by training the transformer on examples of clinical text, such as those given below). In some instances, such a model for generating text embeddings may be trained using transfer learning (e.g., initially trained on another corpus of text, and then receive further training on text related to digital oral care). Some text embeddings may encode text at the word level. Some text embeddings may encode text at the token level. A transformer for generating a text embedding may, in some implementations, be trained, at least in part, with a loss calculation which compares predicted outputs to ground truth outputs (e.g., softmax loss, multiple negatives ranking loss, MSE margin loss, cross-entropy loss or the like). In some instances, the non-text oral care arguments, such as real values or categorical values, may be converted to text, and subsequently embedded using the techniques described herein. In some implementations, oral care arguments 202 may contain natural language text instructions, such as the following, and may (optionally) be encoded into latent representations by latent encoding module 212 (e.g., which may comprise a text transformer, as described herein, among other architectures described herein). The following are examples of natural language instructions that may be provided by a clinician to the generative models described herein, to describe the intended outcome of 3D oral care representation generation using a the denoising diffusion probabilistic models of this disclosure:
1. “Generate a setup to set to Class I molar and canine, 2 mm overbite and add 2 mm of expansion 5-5/5-5.”
2. “Generate a setup to Align with proclination and expansion and finish with 0.5 mm spaces U2-2 for future restorative.”
3. “Generate a bracket setup to finish with 2 mm overbite, 2 mm of overjet, apply Class II elastics and apply L2-2.5 mm incisal to ideal.”
4. “Adjust trimline to meet the gingival margin on all teeth except #8,9 where the trimline should be apical to the gingival margin by 1 mm.”
5. “Adjust the setup to include IPR of 0.3 mm L4-4, retract to close spaces for increased overjet.”
6. “Adjust setup with no second molar movement, rotate upper first molars mesial out for Class I, level lower to a reverse curve of Spee 2 mm, advance the mandible with elastics to Class I canine.”
7. “Generate a setup to intrude posterior teeth to allow for a 2 mm overbite with autorotation, apply lower IPR as needed for overjet, expand and procline for space to align.”
8. “Generate a setup to upright then expand premolars for a broad archform, close midline space with mesial translation, intrude lower 2-2, 2 mm.”
9. “Generate a setup which expands the archform, apply distal crown tip to lower canines until vertical, then apply mesial root tip for a final 10-degree distal tip from vertical.”
10. “Generate a setup which extracts #24 retract and close space to a +1 mm overjet and 2 mm overbite, procline upper 2-2 with 0.5 mm space distal to #8,9.
11. “Generate a restoration design (alternatively a veneer design) which closes the diastema between tooth #8-9 by evenly adding width onto the mesial of both teeth.”
12. “Generate a restoration design (alternatively a veneer design) which adds length onto teeth #6 and #11 so they are the same length as teeth #8 and #9. Add length onto the lateral incisors so they are ½ mm shorter than the centrals.”
13. “Generate a restoration design (alternatively a veneer design) which ensures that the incisal edges of #6-11 form an even semicircle with the incisal edges of the posterior teeth (when looking from the incisal view).”
14. “Generate a restoration design (alternatively a veneer design) for teeth #6-11, making sure that tooth #6 is symmetrical to #11, #7 is symmetrical to #10 and teeth #8-9 are symmetrical.”
15. “Generate a restoration design (alternatively a veneer design) which close the diastemas between lateral incisors and central incisors by making the central incisors' width 70% of the current length. Any space that is left, should be added onto the mesial of the lateral incisors.”
16. “Generate a porcelain crown on #12 that replicates the shape of a first premolar, has tight proximal contacts and has two points of occlusion with the lower tooth. The porcelain crown should have a facial contour that blends from canine to first premolar to second premolar.”
17. “Generate a restoration design (alternatively a veneer design) for the rotated tooth #7 that adds facial bulk so that the width and shape of the tooth are similar to #10.”
18. “Generate a restoration design (alternatively a veneer design) where the facial contour and position of teeth #6, 7, 8, 10, 11 matches the facial position of #9.”
19. “Generate a (zirconia) veneer design where the final color is per recommended shade from the dentist”.
20. “Generate a (zirconia) design where the margins of the veneer fit smoothly and evenly to the margins created on the tooth”.
21. “Generate a (zirconia) veneer design where the veneer shape and contour match the same tooth in the opposite side of the arch, making sure all proximal contacts are closed”.
22. “Generate a customized crown for an upper left central incisor, to be implanted. The crown shape should take into consideration the shape of adjacent teeth and should have no more than x mm (e.g.: 0.1 mm) space between the adjacent teeth.
23. “Generate a missing upper right canine and a missing upper left first molar such as they are placed on the arch and there is no more than x mm (e.g.: 0.05 mm) space/collision with the neighbors' teeth.”

Denoising diffusion probabilistic models are a class of deep generative neural networks which may be trained to generate transformations (e.g., may be used to modify the positions or orientations of 3D representations in 3D space-such as teeth), generate 2D images (e.g., heatmaps or color images), generate 3D representations (e.g., such as point clouds, 3D meshes or voxelized representations), or other 3D oral care representations described herein. Denoising diffusion models may be trained to generate transforms which place the teeth of an arch into poses which are suitable for orthodontic treatment (e.g., intermediate stages or final setups). Denoising diffusion models may be implemented using one or more encoders, one or more MLPs, one or more autoencoders, one or more U-Nets, one or more transformers (e.g., 3D SWIN transformer encoders or 3D SWIN transformer decoders), one or more pyramid encoder-decoders, among other machine learning models. In some implementations, a denoising diffusion model (e.g., such as for setups prediction) may take as input one or more oral care arguments, and also take as input one or more 3D representations of oral care data, such as teeth (e.g., a full arch of segmented teeth in maloccluded poses), appliance components, or fixture model components. The inputted teeth may be in maloccluded poses. In some instances, the maloccluded transforms for the teeth may be provided to the denoising diffusion model. Non-limiting oral care arguments may include a doctor's treatment plan (comprising at least: a set of zero or more procedure parameters, zero or more doctor preferences and zero or more text samples which describe the nature of the intended oral care treatment—such as a final setup or a restoration design generation). Oral care arguments may include real values, categorical values, natural language instructions, among others. In some implementations, a denoising diffusion model may be trained to generate a setup, such as a final setup, which fulfills the specification described by the procedure parameters and/or text. A denoising diffusion model for generating an orthodontic setup may be termed a Diffusion Setups neural network or Diffusion Setups model. A diffusion model which conditions on text may use a neural network to reformat and/or reduce the dimensionality of that text, for example, to generate a latent encoding or latent embedding of the text (e.g., using a transformer or an encoder to generate a text embedding).

A denoising diffusion model may comprise at least one of a forward pass and a reverse pass. The forward pass of the diffusion model may generate training data by iteratively adding noise (e.g., Gaussian noise) to a received 3D oral care representation (e.g., a tooth transform or a point cloud representation of a tooth restoration design). In deployment, the reverse pass of the diffusion model may further operate through an iterative denoising process (e.g., such as using a U-Net trained for the purpose, or other models described herein), which iteratively removes noise from a received 3D oral care representation (e.g., a tooth transform or a point cloud representation a tooth restoration design). Such tooth transforms may define the poses of the teeth in one or more arches. A Diffusion Setups model may generate a 3D oral care representation, such as an orthodontic setup (e.g., for final setups or intermediate staging). During training, a DDM (e.g., diffusion setups or others described herein) may build a training dataset by incrementally adding noise to a latent vector TA that was generated by latent encoding module 214. In deployment, a DDM (e.g., diffusion setups or others described herein) may iteratively denoise one or more latent vectors TB, such as may be generated by a latent encoding module 218. The denoising may be customized, at least in part, by oral care arguments 202, which may undergo optional encoding (212) to generate latent vector TC The latent vectors TA and TB contain reduced-dimensionality information about the one or more 3D oral care representations (e.g., tooth mesh information and/or tooth transform information). When the latent vectors TA or TB correspond to encoded tooth transforms, TA or TB may, in some implementations, be conditioned on (or combined with) latent vectors A which correspond to the one or more 3D representations of teeth. A diffusion model which receives one or more 3D representations of teeth may be trained for generating tooth restoration designs (e.g., such as with GGDM—which may also be trained to generate other types of 3D oral care representations, such as appliance components, fixture model components, transforms, or trimlines). These latent vectors TA or TB may also be conditioned on (or combined with) one or more procedure parameters K and/or one or more doctor preferences L. Such conditioning may be implemented, for example, by concatenating a latent vector TA or TB with K, L, or any of the other oral care arguments described in this disclosure, such as M, N, O, R, S, P, Q, U, V.

Internally to the diffusion model, a latent vector TA (e.g., which may have been generated using an encoder) may undergo many iterations of noise through the course of the forward pass. The series of increasingly noisy versions of TA may be used to train, at least in part, a denoising diffusion neural network (e.g., such as an autoencoder, a U-Net, or others described herein). The latent vector TB may start out as Gaussian noise at the beginning of the reverse pass. Through the several iterations of the diffusion model reverse pass process (e.g., using a trained U-Net, autoencoder, or other model described herein), TA may evolve into a form that may be reconstructed (e.g., using a decoder) into a 3D oral care representation which may be used in oral care appliance generation (e.g., one or more transforms which may place one or more teeth into a setup configuration-such as a final setup or an intermediate stage).

In some implementations, a denoising ML model 210 may use a U-Net architecture with ResNet blocks and self-attention layers as a part of the reverse pass. A ResNet block refers to a residual block, where the activation of one layer in the neural network is forwarded directly to a subsequent deeper layer, with the advantage of enabling deeper networks to be trained. The self-attention layers make use of an attention mechanism which relates different portions of a sequence (i.e., the sequence of intermediate stages, among others) in order to compute a representation of that same sequence. Self-attention is advantageous to intermediate staging prediction, in that each stage of the sequence may be updated or refined with information about other stages in the sequence, as the diffusion model iterates. The denoising ML model 210 (e.g., for setups prediction, or 3D representation generation) may be trained through gradient descent and/or backpropagation. In some implementations, the losses described elsewhere in this disclosure may be used to train, at least in part, the Setups Diffusion model. L1, L2, MSE or other losses described herein may be used to train, at least in part, the denoising ML model 210.

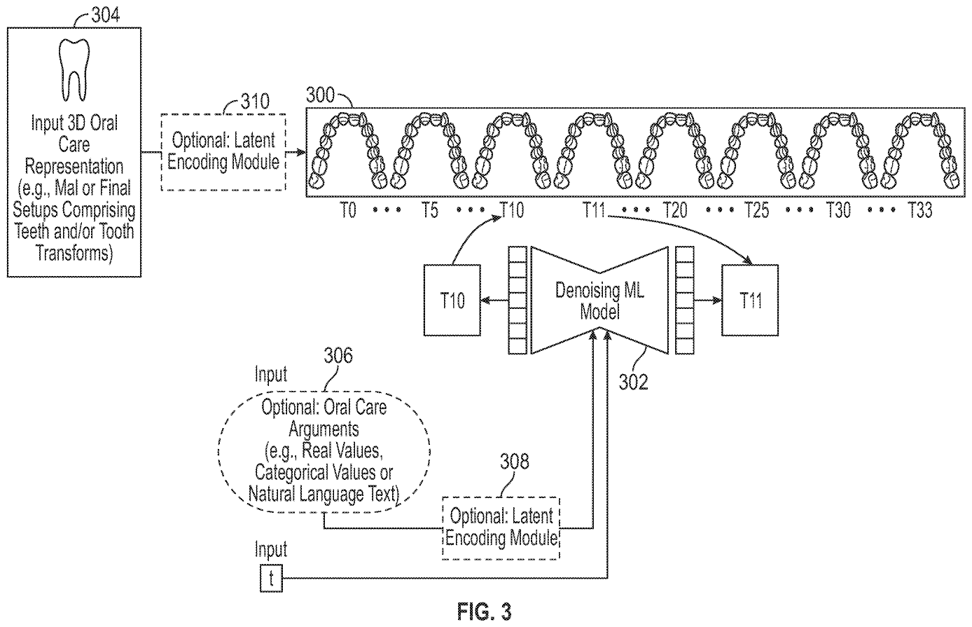

An example of a denoising diffusion model for setups prediction is shown in FIG. 3. FIG. 3 shows how the denoising ML model 302 may be trained on a sequence of increasingly noisy copies of patient case data (e.g., orthodontic setups). The noise may, in some instances, be Gaussian. FIG. 3 shows a Markov chain 300 comprising 34 time points (though other Markov chain sizes are possible). Orthodontic setups 304 (e.g., maloccluded setups, intermediate setups, or final setups) may be provided to Markov chain 300. In some implementations, the orthodontic setups 304 may be encoded (310) into latent form before being provided to the Markov chain 300. Although the Markov chain 300 has length 34, other lengths are possible. In some instances, noise may be added to the patient data starting at TO and proceed for multiple time steps, producing a set of training data with a gradually increasing amount of noise (e.g., setups transforms with more and more noise). Each time point from T0 to T33 may contain transforms (e.g., one transform for each tooth in the arch) and/or 3D representations of teeth. Data (e.g., tooth transforms) from the time points in the Markov chain may be used to train the denoising ML model 302 (e.g., an encoder-decoder network, such as a pyramid encoder-decoder, a 3D SWIN transformer, an autoencoder, or a U-Net) at the center of the reverse pass of the diffusion model. In some implementations, optional oral care arguments 306 (e.g., oral care parameters or oral care metrics) may be provided to the denoising ML model 302. The optional oral care arguments may, in some implementations, be encoded (308) into latent form. Training may proceed over many iterations. A transform may comprise a 4×4 affine transform, although other dimensions are also compatible with the diffusion model-based techniques of this disclosure. For example, a transform may include one or more translation vectors, one or more Euler angles and/or one or more quaternions. The “t” input is a time representation, which may control which time point is sampled during a given timestep of diffusion model training (e.g., the training of the denoising neural network). The input “t” may be used for positional encoding. In some implementations, a time point t may correspond to a stage in orthodontic treatment. The use of positional encoding, similar to that used in transformers, may enable the model to be aware of the relative positions of the stages in the treatment plan sequence. Through this approach, the model may learn how to progress an orthodontic treatment from stage to stage, leveraging what the Diffusion model has learned from prior orthodontic treatment examples in the training dataset.

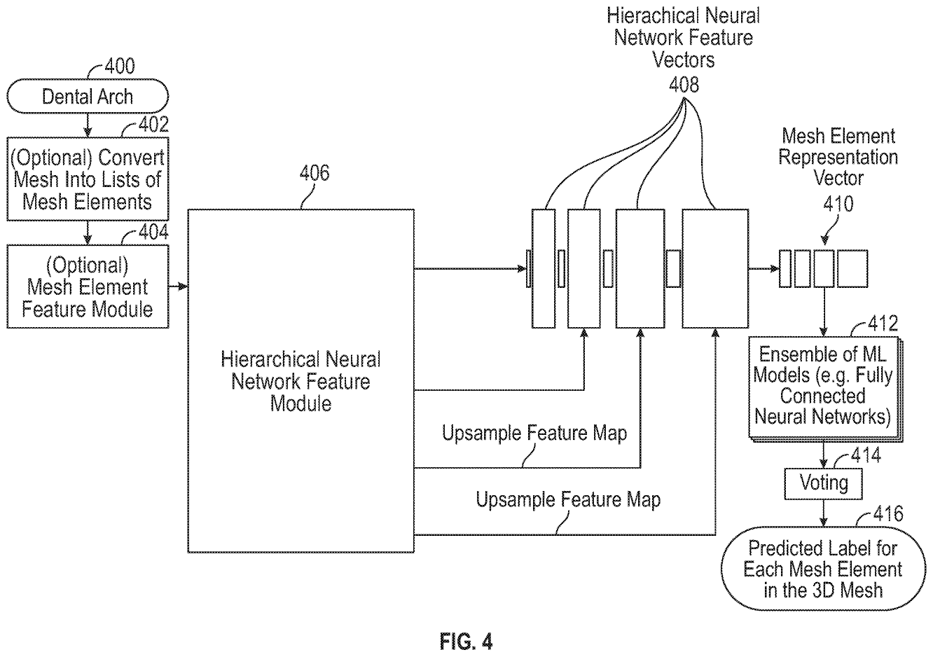

FIG. 4 illustrates an example of a mesh element labelling model which is based on denoising diffusion probabilistic model (DDPM) and which may be used to enable either of the 3D mesh segmentation (e.g., semantic segmentation) and 3D mesh cleanup pipelines. A DDPM for 3D representation segmentation may comprise at least one of a forward pass 206 (e.g., which may involve many steps of adding iteratively more noise to an input 3D representation-which may be used in training an ML model to carry out the reverse process) and a reverse pass 208 (e.g., which may use an ML model, such as a neural network, to iteratively denoise a noisy representation, resulting in a segmentation of the input 3D representation). A DDPM for 3D representation segmentation may, in some implementations, approximate aspects of a Markov process. The HNNFEM 406 described in FIG. 4 may be trained to perform the reverse pass 208. Dental arch data 400 may (optionally) be converted (402) to lists of mesh elements. The mesh elements may undergo optional mesh element feature vector calculation (404). Mesh element features described herein may be provided to the HNNFEM 406, such as mesh element features include: edge mid-point, edge curvature, signed dihedral angles, edge length, edge normal, or others described herein. A DDPM may begin operations on a noisy representation (e.g., a noisy representation of mesh element labels) and may iteratively denoise that representation to produce a denoised representation which may comprise one or more mesh element labels or object masks (alternatively the noisy representation may be directly denoised into one or more segmented 3D representations, such as 3D point clouds or 3D meshes). Formulas which formalize the forward and reverse passes are described herein.

In some implementations, a DDPM for 3D representation segmentation may comprise the following steps: Iteratively add noise (e.g., Gaussian noise) to an input 3D oral care representation, to produce a succession of increasingly noisy versions of that input 3D oral care representation which may then be used in training a denoising neural network of the reverse process. For each 3D representation in a succession of increasingly noisy 3D representations of oral care data: 1) Use the HNNFEM 406 to extract feature maps hierarchical neural network features 408. 2) Collect mesh element-level representations 410 by upsampling the feature maps from the HNNFEM and concatenating those upsampled feature maps into mesh element representation vectors 410. 4) The mesh element representation vectors 410 from step #3 may be used to train one or more ML models for mesh element labeling (e.g., to train an ensemble of neural networks 412 for mesh element labeling). The one or more ML models may vote (414), to determine a final output of mesh element labels 416. The one or more ML models may generate labels for one or more mesh element features. The set of mesh element labels which is generated may, in some implementations, comprise one or more object masks. An object mask may label or otherwise indicate which mesh elements belong to which object in the scene (e.g., which mesh elements belong to an upper left central incisor, to the gums or to a hardware element in the arch mesh from intraoral scanner). FIG. 4 further shows the input of the 3D oral care representation 400 that is to be segmented (e.g., a dental arch), which may (optionally) be arranged (402) into vectors of mesh elements. One or more mesh element features may be computed (404) for each mesh element.

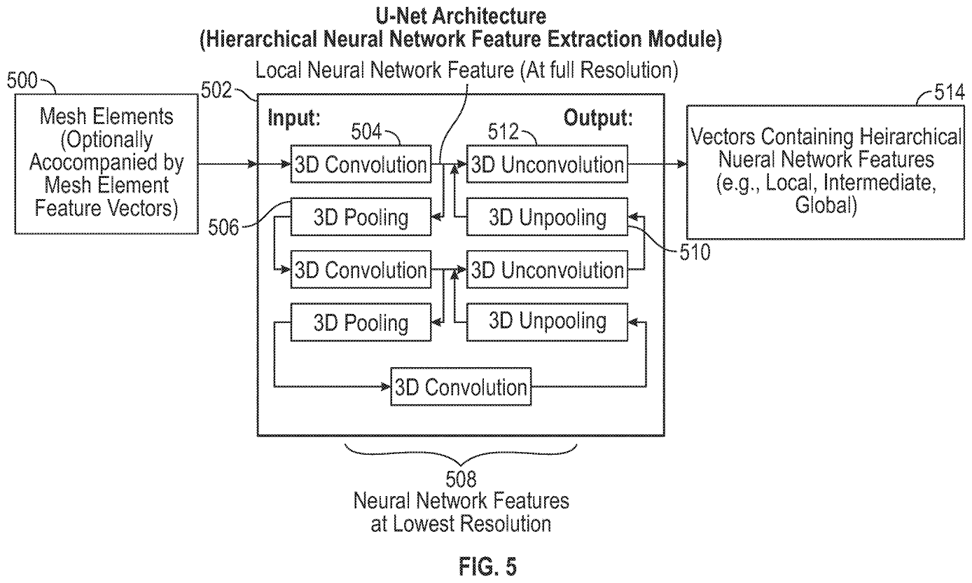

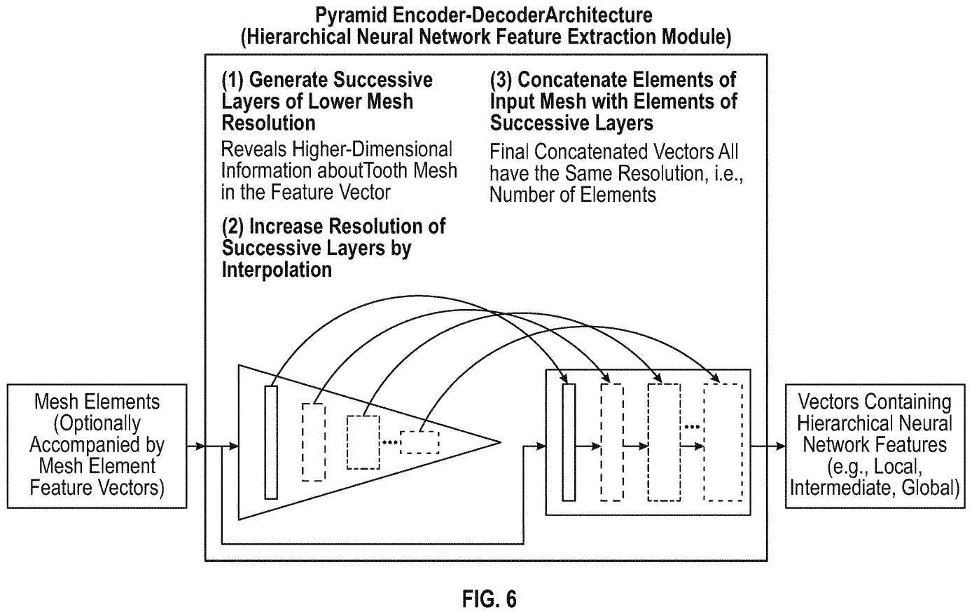

HNNFEM 406 may contain one or more U-Nets 502, one or more pyramid encoder-decoders from FIG. 6, one or more 3D SWIN transformer decoders, or other architectures described herein. In FIG. 5, the mesh elements of 3D representation 500 may be provided to the U-Net 502, which may use a U-shaped structure of convolution 504/unconvolution 512 and/or pooling 506/unpooling operations 510 to extract hierarchical features. The global-most features are extracted at the lowest resolution (508). Vectors 414 or 514 containing hierarchical neural network features may be assembled into mesh element representation vectors 416, and may be provided to an ensemble of ML models 418 (e.g., for resolving mesh element labels). Voting (420) may be performed to resolve ambiguities between the mesh element labels predicted by the ensemble of ML models. The final predicted mesh element labels 422 are sent to the output.

In some implementations (e.g., when a pre-segmentation dental arch is segmented into teeth and gums), training data may include at least a 3D mesh of an arch and the set of mesh element labels (or object masks) which indicates to which tooth each mesh element belongs. This set of mesh element labels (or masks) may undergo the iterative addition of noise, such as in the forward pass shown in FIG. 2. The resulting sets of increasingly noisy mesh element labels may be used as training data to train a denoising diffusion ML model (e.g., such as a U-Net). Such a denoising diffusion ML model may be trained to take as input a noisy (or random) set of mesh element labels (or object masks) for an instant 3D mesh (e.g., of a pre-segmentation dental arch), and gradually, over many iterations, denoise that set of mesh element labels (or object masks). The denoised set of labels (or object masks) may be outputted by the reverse pass in FIG. 2 and used to segment the instant mesh (or point cloud or other 3D representation). In some instances, the set of mesh element labels (or object masks) may undergo encoding using an encoder, which may provide a latent representation to the forward pass. After completion of the reverse pass, in deployment, the denoised representation may undergo reconstruction using a decoder. The mesh element labels may be used to segment the teeth of a pre-segmentation arch. The mesh element labels may, in some instances, be used to label mesh elements as a part of a mesh cleanup operation (e.g., labeling mesh elements for removal, smoothing, scaling or other modifications pursuant to the generation of an artifact for use in digital oral care).

Systems of disclosure may train a denoising diffusion model to segment a 3D representation of oral care data, such as a 3D mesh of the patient's dentition. FIG. 4 pertains to 3D mesh segmentation and 3D mesh cleanup. Both of these techniques share an important attribute, the labelling of 3D mesh elements. In the case of 3D mesh segmentation (e.g., as pertaining to the segmentation of oral care meshes, such as teeth), the various mesh elements of the mesh may be labelled according to which portion of the dental anatomy the mesh elements belong (e.g., gums, upper right central incisor, lower left 2^{nd} bicuspid). Tooth mesh segmentation may label elements according to membership in the various teeth, as designated by one or more of the dental notation systems mentioned herein (e.g., Palmer). In some implementations, for facial-lingual segmentation, each of the mesh elements may be labelled according to membership in either the facial side of the arch or the lingual side of the arch. Other dental anatomy segmentation implementations are possible. In some implementations, oral care arguments 202 may be provided to the denoising diffusion models for segmentation or mesh cleanup described herein, such as decimation factor (or decimation percentage). The decimation factor may control the extent to which pre-segmentation meshes are decimated before mesh element labeling occurs.

The pre-segmentation mesh (e.g., a dental arch produced by an intraoral scanner or CT scanner) may be received by the segmentation system. Mesh element feature vectors may be computed, one or more vectors for each mesh element. One or more mesh element features from elsewhere in this disclosure may be used to form a feature vector for a mesh element. Mesh elements may comprise edges, faces, vertices or voxels (or any combination thereof).

In some implementations, the lists of mesh element feature vectors may be provided to a neural network that refines the mesh element vectors, to extract local and global features from those feature vectors, such as the U-Net structure shown in FIG. 4. A U-Net inside the HNNFEM may comprise 3D mesh convolution and/or subsequent 3D mesh pooling operations, which may serve to reduce the resolution of the mesh and extract neural network features at increasingly global scales. After a succession of such operations, after the global-most neural network features have been extracted, there may be a succession of 3D mesh unpooling and 3D mesh unconvolution operations which return the mesh to the original scale. After the various levels of the U-Net structure have generated outputs, there may be scale-up and concatenation operations. The HNNFEM may generate one or more 3D mesh element-wise feature vectors which may have been scaled-up to the original mesh resolution. These feature vectors may be concatenated and provided as inputs to one or more ML models for mesh element classification or labelling. Any of the supervised ML models disclosed elsewhere in this disclosure may be used for this classification, such as for example SVM or Logistic Regression. In some examples, an ensemble of ML classifiers may take the mesh element representation vectors as inputs and generate mesh element classification labels. In some implementations, an ensemble of fully connected neural networks may be used for such classification. In some cases, a multi-layer perceptron (MLP) may be used for this classification, comprising linear layers and associated ReLU activation functions (e.g., which have the advantage in that not all neurons fire at the same time—which enables a more tailored response to the inputs than activation functions which all fire for every evaluation) and batch normalization operations. Other activation functions are possible, such as the activation functions mentioned elsewhere in this disclosure. Each of the ensemble of ML models may generate a predicted class label for each mesh element. A voting mechanism may then be employed to combine these results and output a final class label prediction for each 3D mesh element. Some implementations may use a transformer to assist in applying labels to mesh elements.

The mesh cleanup and mesh segmentation implementations may, in some instances, differ in the arrangement of the ground truth mesh element labels. In the case of tooth segmentation, ground truth data may be given for each arch mesh that is to be segmented (i.e., each of the teeth in the arch has its mesh elements labeled according to which tooth the mesh element belongs). In turn, such a loss can be computed for facial-lingual segmentation and/or for teeth-gums segmentation (as defined in US Provisional Application No. U.S. 63/366,490). Various of the loss functions of this disclosure may be used to compare the mesh element labels of a predicted segmentation to the mesh element labels of a corresponding ground truth segmentation. Cross-entropy loss is an example of the several candidate loss functions for this comparison. Other possible losses are disclosed elsewhere in this disclosure. In the case of mesh cleanup, ground truth data may be given for each arch mesh that is to be cleaned up. For example, in the case of extraneous material removal, each mesh element corresponding to extraneous material in the arch may be labelled as such, and each mesh element that does not comprise extraneous material (i.e., that is to be retained after mesh cleanup) is labelled as such. In the case of divot removal, each mesh element corresponding to a divot in the arch may be labelled as such, and each mesh element that does not comprise a divot (i.e., that is to be retained after mesh cleanup) is labelled as such.

After the segmentation labels are applied, a process is executed to copy the mesh element of a particular label into a new mesh (aka mesh cutting), and the mesh is saved (e.g., to an electronic storage medium) for further processing. After the mesh cleanup labeling is completed, a process is executed to remove each designated mesh element from the mesh (e.g., using classical mesh processing techniques), and then a process may optionally be executed to fill-in any holes which may have been created by that process (e.g., using the technique described elsewhere in this disclosure which has been trained for mesh in-filling).

Mesh element labeling for mesh segmentation may also be accomplished using an autoencoder which has been trained for the purpose, such as a variational autoencoder, as described elsewhere in this disclosure. More generally, an encoder-decoder network may be trained for mesh element labeling.

In some implementations, the denoising diffusion techniques of this disclosure may be trained to predict archforms. An archform may have a shape which describes aspects of the shape of an arch of teeth. An archform may comprise data structures such as one or more 3D meshes (e.g., such as a mesh which aligns with at least one coordinate axis of at least one tooth's local coordinate system), 3D point clouds, 3D polylines or sets of control points (e.g., control points which define a spline). Such data structures may be generated, for example, using the denoising diffusion method described in FIG. 2.

In some implementations, the denoising diffusion techniques of this disclosure may be trained to generate information pertaining to IPR for use in setups prediction. Information pertaining to IPR may include one or more of 1) an IPR cut surface for a particular tooth, 2) a measure of IPR magnitude to be applied to a side of a particular tooth, 3) a designation of whether a particular tooth is to undergo IPR—including an indication of whether IPR is to be applied to the mesial or distal side of the tooth, or 4) a designation of one or more stages in which a particular tooth is to undergo IPR. The training data for such a model may be generated, at least in part, by iteratively adding noise to 1) representations of one or more IPR cut surfaces from the cases in the training dataset, 2) representations of one or more teeth or other aspects of the patient's detention, or 3) one or more tooth transforms. An ML model (e.g., a U-Net) may be trained to iteratively denoise an initial representation of one or more such examples of training data (e.g., IPR cut surfaces may be initialized, at least in part, randomly or are initialized using default values). Upon completion of the reverse pass 208, one or more generated (or modified) data 220 pertaining to IPR may be outputted. For example, IPR cut surfaces may be generated by the denoising diffusion techniques described herein. In some implementations, a denoising diffusion model may be trained to render a determination of whether a target tooth is accessible for IPR. Information pertaining to IPR may be generated, for example, using the denoising diffusion method described in FIG. 2. For example, the method in FIG. 2 may predict an orthodontic setup which has optional associated IPR information. For each tooth, a setup transform may be generated, along with one or more associated items of IPR information (e.g., one or more IPR cut surfaces, a flag to indicate whether IPR is to be performed, a list of states in which to perform IPR, etc.).

In some implementations, techniques of this disclosure may be trained to predict trimlines. A trimline may define a cutting path to remove a thermoformed tray from a fixture model (e.g., a tray for orthodontic aligner treatment or an indirect bonding tray to deliver brackets to teeth for orthodontic treatment). One or more polylines (or meshes or point clouds) may be used to define a trimline. Such polylines (or meshes or point clouds) may be generated, for example, using the denoising diffusion method described in FIG. 2.

In some implementations, the denoising diffusion techniques of this disclosure may be trained to predict one or more local orthogonal coordinate axes for a tooth (e.g., such as to predict one or more of X, Y and Z orthogonal axes for a tooth). In other implementations, the denoising diffusion techniques of this disclosure may be trained to predict one or more archform coordinate axes. A position along an archform coordinate axis may comprise a tuple [1, d, e] relative to a reference archform spline S which approximates the shape of an arch of teeth. A rotation may comprise a tuple [a, b, g] which stands for alpha, beta and gamma rotations. Alpha describes a rotation around the 1-axis. Beta describes a rotation around the d-axis. Gamma describes a rotation around the e-axis. A full tuple to describe position and rotation may comprise [1, d, e, a, b, g]. p is a point along S with arch length 1. d is the distance between a tooth origin t and the reference archform spline S. The tooth origin t is obtained by translating up along the d axis by a distance ‘d’, and then translating along the e-axis by a distance ‘e’. The e-axis is perpendicular to the d-axis and the 1-axis and may be defined to come out of the page or into the page. e stands for eminence. 1 stands for the length across the archform spline. d stands for the distance away from the archform spline. Such an archform coordinate system is described by U.S. Published Patent Application US20210259808A1, by same applicant, the entirety of which is incorporated herein by reference. The coordinate systems described herein may facilitate later automation for oral care treatment of the patient, such as automated setups prediction. One or more transforms, vectors or tuples may be used to define a coordinate system (or to define a pose within a coordinate system). Such transforms (or vectors or tuples) may be generated, for example, using the denoising diffusion method described in FIG. 2.

Table 3 describes the input data and generated data for several non-limiting examples of the generative techniques described herein. Denoising diffusion models described herein may, in some implementations, be trained for the generation (or modification) of the input data in Table 3, yielding the generated data in Table 3.

Techniques of this disclosure may be trained to generate (or modify) point clouds (e.g., where a point may be described as a ID vector-such as (x, y, z)), polylines (points connected in order by edges), 3D meshes (points connected via edges to form faces), splines (which may be computed through a set of generated control points), sparse voxelized representations (which may be described as a set of points corresponding to the centroid of each voxel or to some other landmark of the voxel—such as the boundary of the voxel), one or more transforms (which may take the form of one or more ID vectors or one or more 2D matrices—such as a 4×4 matrix) or the like. In some implementations, a voxelized representation may be computed from a 3D point cloud or a 3D mesh. In some implementations, a 3D point cloud may be computed from a voxelized representation. In some implementations, a 3D mesh may be computed from a 3D point cloud.

TABLE 3

| Digital oral | | |
|---|---|---|
| care technique | Input data | Generated data |
| Segmentation | 3D representation of patient's pre- | One or more mesh element labels for one or |
| | segmentation dentition (e.g., a | more aspects of the patient's dentition. A label |
| | mesh including teeth and gums) | may indicate which mesh elements should be |
| | | included in which segmented portion of the |
| | | patient's dentition. |
| Coordinate | One or more (segmented) teeth | Coordinate system for one or more teeth (or |
| system | | an arch). May comprise one or more |
| | | transforms. |
| Mesh cleanup | 3D representation of patient's | One or more mesh element labels for one or |
| | dentition (e.g., a mesh including | more aspects of the patient's dentition. A label |
| | teeth and gums) | may flag a mesh element for removal or |
| | | modification. |
| Restoration design | 3D representation of patient's pre- | 3D representation of patient's intended post- |
| generation | restoration tooth | restoration tooth |
| CTA trimline | 3D representation of patient's | 3D representation of trimline (e.g., 3D mesh |
| | dentition (e.g., a mesh including | or 3D polyline) |
| | teeth and gums) | |
| CTA setups | Two or more tooth meshes and/or | Transforms for one or more teeth |
| (for final setups | tooth transforms. Tooth meshes | |
| or intermediate | may be in their maloccluded poses. | |
| staging) | Transforms may correspond to | |
| | maloccluded poses. | |
| Hardware (e.g., | One or more (segmented) teeth | Transform for placement of hardware relative |
| bracket/attachment) | | to the one or more teeth |
| placement | | |
| Archform | 3D representation of patient's | 3D polyline or a 3D mesh or surface, that |
| generation | dentition (e.g., a mesh including | describes the contours or layout of an arch of |
| | teeth and gums). May comprise | teeth |
| | one or more segmented teeth. | |
| Generated oral | 3D representation of patient's | One or more oral care appliance components |
| care appliance | dentition (e.g., a mesh including | with shape and/or structure that is customized |
| component | teeth and gums). May comprise | to aspects of the patient's dentition |
| (e.g., for dental | one or more segmented teeth. | |
| restoration) | | |
| Fixture model | 3D representation of the patient's | 3D representation of the patient's dentition |
| component | dentition | with one or more fixture model components |
| generation | | attached |
| Placed oral care | 3D representation of patient's | One or more transforms which place a library |
| appliance | dentition (e.g., a mesh including | component relative to aspects of the patient's |
| component | teeth and gums). May comprise | dentition |
| (e.g., for dental | one or more segmented teeth. | |
| restoration) | | |

In some implementations, an automated setups prediction model (e.g., a denoising diffusion probabilistic model) may be trained to generate a setup with a customized curve-of-spee (e.g., a curve-of-spee which conforms to the intended outcome of the treatment of the patient). Such a model may be trained on cohort patient case data. One or more oral care metrics may be computed on each case to quantify or measure aspects of that case's curve-of-spee. At training time, one or more of such metrics may be provided to the setups prediction model, for example, to influence the model regarding the geometry and/or structure of each case's curve-of-spee. Upon deployment of the setups prediction model, that same input pathway to the trained neural network may be configured with one or more values as instructions to the model about an intended curve-of-spee. Such values may automatically generate a setup with a curve-of-spee which meets the aesthetic and/or medical treatment needs of the particular patient case.

In some implementations, a curve-of-spee metric may measure the curvature of the occlusal or incisal surfaces of the teeth on either the left or right sides of the arch, with respect to the occlusal plane. The occlusal plane may, in some instances, be computed as a surface which averages the incisal or occlusal surfaces of the teeth (for one or both arches). In some implementations, a curvature metric may be computed along a normal vector, such as a vector which is normal to the occlusal plane. In other implementations, a curvature metric may be computed along the normal vector of another plane. In some implementations, an XY plane may be defined to correspond to the occlusal plane. An orthogonal plane may be defined as the plane that is orthogonal to the occlusal plane, which also passes through a curve-of-spee line segment, where the curve-of-spee line segment is defined by a first endpoint which is a landmarking point on a first tooth (e.g., canine) and a second endpoint which is a landmarking point on the most-posterior tooth of the same side of the arch. A landmarking point can in some implementations be located along the incisal edge of a tooth or on the cusp of a tooth. In some instances, the landmarking points for the intermediate teeth (e.g., teeth which are located between the first tooth and the most posterior tooth) on either the left or right sides of the arch may form a curved path, such as may be described by a polyline. The following is a non-limiting list of curve-of-spee oral care metrics.
1) Measure the vertical height between a line segment and a point. Stated another way, measure a distance between a line segment and a point along the z-axis. The line segment is defined by joining the highest cusp of the most-posterior tooth (in the lower arch) and the cusp of the first tooth on that side (in the lower arch). Given the subset of teeth between the first tooth and the most-posterior tooth, the point is defined by the highest cusp of the lowest tooth of this subset. Stated another way, a curve-of-spee metric may be computed using the following 4 steps. i) Line: Form a line between the highest cusp on the most posterior tooth and the cusp of the first tooth. ii) Curve_Point_A: Given the set of teeth between the most posterior tooth and the first tooth, find the highest point of the lowest tooth. iii) Curve_Point_B: Project Curve_Point_A onto the Line to find a point (Curve_Point_B) along the line that is closest to Curve_Point_A. iv) Curve-Of-Spee: Find the height difference between Curve_Point B and Curve_Point_A.
2) Project one or more intermediate landmark points (e.g., points on the teeth which lie between the first tooth and the most-posterior tooth on that side of the arch) and the curve-of-spee line segment onto the orthogonal plane. Compute the curve-of-spee metric by measuring the distance between the farthest of the projected intermediate points to the projected curve-of-spee line segment. This yields a measure for the curvature of the arch relative to the orthogonal plane.
3) Project one or more intermediate landmark points and the curve-of-spee line segment onto the occlusal plane. Compute Curve of Spee in this plane by measuring the distance between the farthest of the projected intermediate points to the projected curve-of-spee line segment. This yields a measure for the curvature of the arch relative to the occlusal plane.
4) Skip the projection and compute the distances and curvatures in the 3D space. Compute Curve of Spee by measuring the distance between the farthest of the intermediate points to the curve-of-spee line segment. This yields a measure for the curvature of the arch in 3D space.
5) Compute the slope of the projected curve-of-spee line segment on the occlusal plane.
6) Compute the slope of the projected curve-of-spee line segment in the orthogonal plane.

Curve-of-spee metrics 5 and 6 may help the network to reduce some more degrees of freedom in defining how the patient's arch is curved in the posterior of the mouth.

Techniques described herein (e.g., denoising diffusion probabilistic models) may be trained to generate transforms which may place the patient's teeth into poses suitable for use in orthodontic setups (e.g., intermediate stages or final setups), according to the specification of the oral care arguments which may, in some implementations, be provided to the generative model. Optional oral care arguments 202 may be provided to the denoising ML model 210 (e.g., a U-Net) which is trained to perform the reverse pass 220. Oral care arguments may include oral care parameters as disclosed herein, or other real-valued, text-based or categorical inputs which specify intended aspects of the one or more 3D oral care representations which are to be generated. In some instances, oral care arguments may include oral care metrics, which may describe intended aspects of the one or more 3D oral care representations which are to be generated. Oral care arguments are specifically adapted to the implementations described herein. For example, the oral care arguments may specify the intended the designs (e.g., including shape and/or structure) of 3D oral care representations which may be generated (or modified) according to techniques described herein. In short, implementations using the specific oral care arguments disclosed herein generate more accurate 3D oral care representations than implementations that do not use the specific oral care arguments. In some instances, a text encoder may encode a set of natural language instructions from the clinician (e.g., generate a text embedding). A text string may comprise tokens. An encoder for generating text embeddings may, in some implementations, apply either mean-pooling or max-pooling between the token vectors. In some instances, a transformer (e.g., BERT or Siamese BERT) may be trained to extract embeddings of text for use in digital oral care (e.g., by training the transformer on examples of clinical text, such as those given below). In some instances, such a model for generating text embeddings may be trained using transfer learning (e.g., initially trained on another corpus of text, and then receive further training on text related to digital oral care). Some text embeddings may encode text at the word level. Some text embeddings may encode text at the token level. A transformer for generating a text embedding may, in some implementations, be trained, at least in part, with a loss calculation which compares predicted outputs to ground truth outputs (e.g., softmax loss, multiple negatives ranking loss, MSE margin loss, cross-entropy loss or the like). In some instances, the non-text arguments, such as real values or categorical values, may be converted to text, and subsequently embedded using the techniques described herein. The following are examples of natural language instructions that may be issued by a clinician to the generative models described herein: “Generate a setup to set to Class I molar and canine, 2 mm overbite and add 2 mm of expansion 5-5/5-5”, “Generate a setup to align with proclination and expansion and finish with 0.5 mm spaces U2-2 for future restorative”, or “Adjust the setup with no second molar movement, rotate upper first molars mesial out for Class I, level lower to a reverse curve of Spee 2 mm, advance the mandible with elastics to Class I canine.”

Techniques of this disclosure may, in some implementations, use PointNet, PointNet++, or derivative neural networks (e.g., networks trained via transfer learning using either PointNet or PointNet++as a basis for training) to extract local or global neural network features from a 3D point cloud or other 3D representation (e.g., a 3D point cloud describing aspects of the patient's dentition-such as teeth or gums). Techniques of this disclosure may, in some implementations, use U-Nets to extract local or global neural network features from a 3D point cloud or other 3D representation.

3D oral care representations are described herein as such because 3-dimensional representations are currently state of the art. Nevertheless, 3D oral care representations are intended to be used in a non-limiting fashion to encompass any representations of 3-dimensions or higher orders of dimensionality (e.g., 4D, 5D, etc.), and it should be appreciated that machine learning models can be trained using the techniques disclosed herein to operate on representations of higher orders of dimensionality.

In some instances, input data may comprise 3D mesh data, 3D point cloud data, 3D surface data, 3D polyline data, 3D voxel data, or data pertaining to a spline (e.g., control points). An encoder-decoder structure may comprise one or more encoders, or one or more decoders. In some implementations, the encoder may take as input mesh element feature vectors for one or more of the inputted mesh elements. By processing mesh element feature vectors, the encoder is trained in a manner to generate more accurate representations of the input data. For example, the mesh element feature vectors may provide the encoder with more information about the shape and/or structure of the mesh, and therefore the additional information provided allows the encoder to make better-informed decisions and/or generate more-accurate latent representations of the mesh. Examples of encoder-decoder structures include U-Nets, autoencoders or transformers (among others). A representation generation module may comprise one or more encoder-decoder structures (or portions of encoders-decoder structures-such as individual encoders or individual decoders). A representation generation module may generate an information-rich (optionally reduced-dimensionality) representation of the input data, which may be more easily consumed by other generative or discriminative machine learning models.

A U-Net may comprise an encoder, followed by a decoder. The architecture of a U-Net may resemble a U shape. The encoder may extract one or more global neural network features from the input 3D representation, zero or more intermediate-level neural network features, or one or more local neural network features (at the most local level as contrasted with the most global level). The output from each level of the encoder may be passed along to the input of corresponding levels of a decoder (e.g., by way of skip connections). Like the encoder, the decoder may operate on multiple levels of global-to-local neural network features. For instance, the decoder may output a representation of the input data which may contain global, intermediate or local information about the input data. The U-Net may, in some implementations, generate an information-rich (optionally reduced-dimensionality) representation of the input data, which may be more easily consumed by other generative or discriminative machine learning models.

An autoencoder may be configured to encode the input data into a latent form. An autoencoder may train an encoder to reformat the input data into a reduced-dimensionality latent form in between the encoder and the decoder, and then train a decoder to reconstruct the input data from that latent form of the data. A reconstruction error may be computed to quantify the extent to which the reconstructed form of the data differs from the input data. The latent form may, in some implementations, be used as an information-rich reduced-dimensionality representation of the input data which may be more easily consumed by other generative or discriminative machine learning models. In most scenarios, an autoencoder may be trained to input a 3D representation, encode that 3D representation into a latent form (e.g., a latent embedding), and then reconstruct a close facsimile of that input 3D representation as the output.

A transformer may be trained to use self-attention to generate, at least in part, representations of its input. A transformer may encode long-range dependencies (e.g., encode relationships between a large number of inputs). A transformer may comprise an encoder or a decoder. Such an encoder may, in some implementations, operate in a bi-directional fashion or may operate a self-attention mechanism. Such a decoder may, in some implementations, may operate a masked self-attention mechanism, may operate a cross-attention mechanism, or may operate in an auto-regressive manner. The self-attention operations of the transformers described herein may, in some implementations, relate different positions or aspects of an individual 3D oral care representation in order to compute a reduced-dimensionality representation of that 3D oral care representation. The cross-attention operations of the transformers described herein may, in some implementations, mix or combine aspects of two (or more) different 3D oral care representations. The auto-regressive operations of the transformers described herein may, in some implementations, consume previously generated aspects of 3D oral care representations (e.g., previously generated points, point clouds, transforms, etc.) as additional input when generating a new or modified 3D oral care representation. The transformer may, in some implementations, generate a latent form of the input data, which may be used as an information-rich reduced-dimensionality representation of the input data, which may be more easily consumed by other generative or discriminative machine learning models.

In some implementations, an encoder-decoder structure may first be trained as an autoencoder. In deployment, one or more modifications may be made to the latent form of the input data. This modified latent form may then proceed to be reconstructed by the decoder, yielding a reconstructed form of the input data which differs from the input data in one or more intended aspects. Oral care arguments, such as oral care parameters or oral care metrics may be supplied to the encoder, the decoder, or may be used in the modification of the latent form, to influence the encoder-decoder structure in generating a reconstructed form that has desired characteristics (e.g., characteristics which may differ from that of the input data).

Techniques of this disclosure may, in some instances, be trained using federated learning. Federated learning may enable multiple remote clinicians to iteratively improve a machine learning model (e.g., validation of 3D oral care representations, mesh segmentation, mesh cleanup, other techniques which involve labeling mesh elements, coordinate system prediction, non-organic object placement on teeth, appliance component generation, tooth restoration design generation, techniques for placing 3D oral care representations, setups prediction, generation or modification of 3D oral care representations using autoencoders, generation or modification of 3D oral care representations using transformers, generation or modification of 3D oral care representations using diffusion models, 3D oral care representation classification, imputation of missing values), while protecting data privacy (e.g., the clinical data may not need to be sent “over the wire” to a third party). Data privacy is particularly important to clinical data, which is protected by applicable laws. A clinician may receive a copy of a machine learning model, use a local machine learning program to further train that ML model using locally available data from the local clinic, and then send the updated ML model back to the central hub or third party. The central hub or third party may integrate the updated ML models from multiple clinicians into a single updated ML model which benefits from the learnings of recently collected patient data at the various clinical sites. In this way, a new ML model may be trained which benefits from additional and updated patient data (possibly from multiple clinical sites), while those patient data are never actually sent to the 3rd party. Training on a local in-clinic device may, in some instances, be performed when the device is idle or otherwise be performed during off-hours (e.g., when patients are not being treated in the clinic). Devices in the clinical environment for the collection of data and/or the training of ML models for techniques described herein may include intra-oral scanners, CT scanners, X-ray machines, laptop computers, servers, desktop computers or handheld devices (such as smart phones with image collection capability). In addition to federated learning techniques, in some implementations, contrastive learning may be used to train, at least in part, the ML models described herein. Contrastive learning may, in some instances, augment samples in a training dataset to accentuate the differences in samples from different classes and/or increase the similarity of samples of the same class.

In some instances, a local coordinate system for a 3D oral care representation, such as a tooth, may be described by one or more transforms (e.g., an affine transformation matrix, translation vector or quaternion). Systems of this disclosure may be trained for coordinate system prediction using past cohort patient case data. The past patient data may include at least: one or more tooth meshes or one or more ground truth tooth coordinate systems. Machine learning models such as: U-Nets, encoders, autoencoders, pyramid encoder-decoders, transformers, or convolution and/or pooling layers, may be trained for coordinate system prediction. Representation learning may determine a representation of a tooth (e.g., encoding a mesh or point cloud into a latent representation, for example, using a U-Net, encoder, transformer, convolution and/or pooling layers or the like), and then predict a transform for that representation (e.g., using a trained multilayer perceptron, transformer, encoder, transformer, or the like) that defines a local coordinate system for that representation (e.g., comprising one or more coordinate axes). In the instance where the coordinate system is predicted for a tooth mesh, the mesh convolutional techniques described herein can leverage invariance to rotations, translations, and/or scaling of that tooth mesh to generate predications that techniques that are not invariant to the rotations, translations, and/or scaling of that tooth mesh cannot generate. Pose transfer techniques may be trained for coordinate system prediction, in the form of predicting a transform for a tooth. Reinforcement learning techniques may be trained for coordinate system prediction, in the form of predicting a transform for a tooth.

Machine learning models such as: U-Nets, encoders, autoencoders, pyramid encoder-decoders, transformers, or convolution and/or pooling layers, may be trained as a part of a method for hardware (or appliance component) placement. Representation learning may train a first module to determine an embedded representation of a 3D oral care representation (e.g., encoding a mesh or point cloud into a latent form using an autoencoder, or using a U-Net, encoder, transformer, block of convolution and/or pooling layers or the like). That representation may comprise a reduced dimensionality form and/or information-rich version of the inputted 3D oral care representation. In some implementations, the generation of a representation may be aided by the calculation of a mesh element feature vector for one or more mesh elements (e.g., each mesh element). In some implementations, a representation may be computed for a hardware element (or appliance component). Such representations are suitable to be provided to a second module, which may perform a generative task, such as transform prediction (e.g., a transform to place a 3D oral care representation relative to another 3D oral care representation, such as to place a hardware element or appliance component relative to one or more teeth) or 3D point cloud generation. Such a transform may comprise an affine transformation matrix, translation vector or quaternion or the like. Machine learning models which may be trained to predict a transform to place a hardware element (or appliance component) relative to elements of patient dentition include: MLP, transformer, encoder, or the like. Systems of this disclosure may be trained for 3D oral care appliance placement using past cohort patient case data. The past patient data may include at least: one or more ground truth transforms and one or more 3D oral care representations (such as tooth meshes, or other elements of patient dentition). In the instance where a U-Net (among other neural networks) is trained to generate the representations of tooth meshes, the mesh convolution and/or mesh pooling techniques described herein leverage invariance to rotations, translations, and/or scaling of that tooth mesh to generate predications that techniques that are not invariant to the rotations, translations, and/or scaling of that tooth mesh cannot generate. Pose transfer techniques may be trained for hardware or appliance component placement. Reinforcement learning techniques may be trained for hardware or appliance component placement.

The denoising diffusion techniques (e.g., the method in FIG. 2) described herein may, in some implementations, trained to fill-in missing aspects of a 3D representation of a patient's dentition (e.g., of a segmented tooth or of a digital fixture model or digital fixture model component). In some examples, a 3D scan of a tooth crown (or fixture model or other 3D oral care representations described herein) may be missing some portions of one or more surfaces (e.g., due to occlusion by surrounding teeth during intraoral scanning). A denoising diffusion model may generate training data by iteratively degrading or adding noise to a 3D representation of a tooth (e.g., by removing patches or portions of the surface of the tooth crown or tooth root). A sequence of progressively nosier versions of the 3D representant of a tooth from the training dataset may be generated. Noise may be added to a point cloud/mesh/voxelized representation of the tooth by 1) adding random translation to one or more mesh elements, 2) applying small random scaling or distortion to aspects of the tooth or 3) by removing mesh elements (e.g., creating holes), among other methods. This sequence of progressively noisier versions of the tooth may then be used to train a denoising diffusion neural network (e.g., a U-Net or others encoder-decoder structures-such as pyramid encoders-decoders, transformers or autoencoders) to reconstruct (e.g., in small steps) a noisy tooth. A noisy set of mesh elements (e.g., initialized from a tooth which has missing data) may be provided to the denoising diffusion neural network, and the denoising diffusion neural network may be trained to iteratively remove noise and/or repair the surface of the tooth. After multiple iterations of incremental repairs, the restored tooth (or other 3D oral care representation) may be outputted by the denoising diffusion model and used in later digital oral care processing to generation an oral care appliance.

Fixture model components may be generated or modified using the denoising diffusion techniques of this disclosure (e.g., the method in FIG. 2). A digital fixture model may comprise 3D representations of the patient's dentition, with optional fixture model components attached to that dentition. 3D representation generation techniques of this disclosure (e.g., denoising diffusion techniques to generate 3D point clouds, 3D voxelized representations, or other representations) may be trained to generate (or modify) aspects of a digital fixture model, to include treatment-enhancing fixture model components. Fixture model components may include 3D representations (e.g., 3D point clouds, 3D meshes, or voxelized representations) of one or more of the following non-limiting items: 1) interproximal webbing-which may fill-in space or smooth-out the gaps between teeth to ensure aligner removability. 2) blockout-which may be added to the fixture model to remove overhangs that might interfere with plastic tray thermoforming or to ensure aligner removability. 3) bite blocks—occlusal features on the molars or premolars intended to prop the bite open. 4) bite ramps—lingual features on incisors and cuspids intended to prop the bite open. 5) interproximal reinforcement—a structure on the exterior of an oral care appliance (e.g., an aligner tray), which may extend from a first gingival edge of the appliance body on a labial side of the appliance body along an interproximal region between the first tooth and the second tooth to a second gingival edge of the appliance body on a lingual side of the appliance body. The effect of the interproximal reinforcement on the appliance body at the interproximal region may be stiffer than a labial face and a lingual face of the first shell. This may allow the aligner to grasp the teeth on either side of the reinforcement more firmly. 6) gingival ridge—a structure which may extend along the gingival edge of a tooth in the mesial-distal direction for the purpose of enhancing engagement between the aligner and a given tooth. 7) torque points—structures which may enhance force delivered to a given tooth at specified locations. 8) power ridges—structures which may enhance force delivered to a given tooth at a specified location. 9) dimples—structures which may enhance force delivered to a given tooth at specified locations. 10) digital pontic tooth—structure which may hold space open or reserve space in an arch for a tooth which is partially erupted, or the like. In aligners, a physical pontic is a tooth pocket that does not cover a tooth when the aligner is installed on the teeth. The tooth pocket may be filled with tooth-colored wax, silicone, or composite to provide a more aesthetic appearance. 11) power bars—blockout added in an edentulous space to provide strength and support to the tray. A power bar may fill-in voids. Abutments or healing caps may be blocked-out with a power bar. 12) trim line—digital path along the digital fixture model, which may approximately follow the contours of the gingiva (e.g., may be biased 1 or 2 mm in the gingival direction). The trimline may define the path along which a clear aligner may be cut or separated from a physical fixture model, after 3D printing. 13) undercut fill—material which is added to the fixture model to avoid the formation of cavities between the fixture model's height of contour and another boundary (e.g., the gingiva or the plane that the plane that undergirds the physical fixture model after 3D printing).

Techniques of this disclosure may also generate (or modify) other geometries that intrude on the tooth pocket or reinforce an oral care appliance (e.g., an orthodontic aligner tray). Techniques of this disclosure may, in some implementations, determine the location, size, and/or shape of a fixture model component to produce a desired treatment outcome.

Interproximal webbing may be generated using techniques of this disclosure, for example during the fixture model quality control phase of orthodontic aligner fabrication. Interproximal webbing is material (e.g., which may comprise mesh elements, such as vertices/edges/faces, among others) that smooths-out or creates a positive fill in the interproximal areas of the fixture model. Interproximal webbing is extra material added to the interproximal areas of the teeth in a digital fixture model (e.g., which may be 3D printed and rendering in physical form) to reduce the tendency of the aligner, retainer, attachment template, or bonding tray to lock onto the physical fixture model during the orthodontic aligner thermoforming process. Interproximal webbing may improve the functioning of the aligner tray by improving the ability of the tray to slide off of the fixture model after thermoforming, or by improving the fit of the tray onto the patient's teeth (e.g., making the tray easier to insert onto the teeth or to remove from the teeth).

Blockout (e.g., which may comprise mesh elements, such as vertices/edges/faces, among others) may be generated using techniques of this disclosure. Blockout is material which may be added to undercut regions of a digital fixture model, so that the aligner, retainer, attachment template, or bonding tray, 3D printed mold, or other oral care appliance, from locking onto the physical fixture model during the orthodontic aligner thermoforming process. Block out may be generated to fill-in a portion of a digital fixture model with an undercut, so that a thermoformed aligner tray does not grab onto that undercut. Blockout may improve the functioning of the aligner tray by improving the ability of the tray to slide off of the fixture model after thermoforming. Blockout may facilitate later thermoforming (e.g., help avoid the aligner tray getting stuck on the physical fixture model).

Pontic tooth designs may be generated using techniques of this disclosure. A pontic tooth is a digital 3D representation of a tooth which may act as a placeholder in an arch. In an aligner, a pontic may function as a tooth pocket that may be filled with a tooth-colored material (e.g., wax) to improve aesthetics. A pontic tooth may hold a space in the arch open during orthodontic setups generation. When automated setups prediction is performed, transforms for intermediate stages may be generated. One or more pontic teeth may be defined to hold open space for missing teeth or act as a placeholder as space closes or opens in the arch during setups predicted staging or an unerupted tooth may erupt into a space where a pontic is present. Creating a pocket for the tooth to erupt into.

Digital pontic teeth may be placed in (or generated within) an arch (e.g., during setups generation or fixture model generation) to reserve space in an arch for missing or extracted teeth (e.g., so that adjacent teeth do not encroach upon that space over the course of successive intermediate stages of orthodontic treatment). In some implementations, digital pontic tooth may be used when the space (e.g., the space that is to be held open) is at least a threshold dimension (e.g., a width of 4 mm, among others). In some instances, digital pontic teeth for UL4-UR4 or LL4-LR4 may be placed (or generated or modified) when space is available or when there is a partially erupted tooth within the space. When there is a partially erupted tooth in the space, a digital pontic tooth may be placed over the erupting tooth to maintain a space for the erupting tooth to erupt into. Pontic teeth may be placed (or generated or modified) to be inside the gingiva, to minimize (or avoid) heavy occlusal contacts (e.g., contacts between the chewing surfaces of the upper or lower arches), or to cover an erupting tooth (when present), among other conditions.

Aspects of the present disclosure can provide a technical solution to the technical problem of generating, using denoising diffusion models, 3D oral care representations. 3D oral care representations which may be generated include: mesh element labels for segmentation or mesh cleanup, transforms for use in setups generation (or fixture model or appliance generation), coordinate systems (e.g., for use as local coordinate systems of teeth), 3D point clouds (or other 3D representations described herein) that describe teeth (or appliance components or fixture model components), or others described herein. In particular, by practicing techniques disclosed herein computing systems specifically adapted to generate 3D oral care representations for use in oral care appliance generation are improved. For example, aspects of the present disclosure improve the performance of a computing system having a 3D representation of the patient's dentition by reducing the consumption of computing resources. In particular, aspects of the present disclosure reduce computing resource consumption by encoding inputs (e.g., 3D representations of teeth, mesh element labels, tooth transforms, coordinate systems, 3D representations of appliance components, 3D representations of fixture model components, or other 3D oral care representations described herein) into reduced dimensionality latent representations (e.g., using the encoder portion of a reconstruction autoencoder). The methods may potentially reduce thousands of mesh elements (each of which may be described by several real numbers) into a latent vector of potentially hundreds of real numbers, so that computing resources are not unnecessarily wasted by processing excess quantities of data. Additionally, encoding the input data into latent representations does not reduce the overall predictive accuracy of the computing system (and indeed may actually improve predictions because the input provided to the ML model after the encoding is a more accurate (or better) representation of the patient's dentition). For example, the latent representations may remove noise or other artifacts which are unimportant (and which may reduce the accuracy of the predictive models) and provide a distilled set of values that accurately describe the 3D oral care representation that is encoded. That is, aspects of the present disclosure provide for more efficient allocation of computing resources, and in a way that improves the accuracy of the underlying system.

Furthermore, aspects of the present disclosure may need to be executed in a time-constrained manner, such as when an oral care appliance must be generated for a patient immediately after intraoral scanning (e.g., while the patient waits in the clinician's office). As such, aspects of the present disclosure are necessarily rooted in the underlying computer technology of 3D oral care representation generation using denoising diffusion models and cannot be performed by a human, even with the aid of pen and paper. For instance, implementations of the present disclosure must be capable of: 1) storing thousands or millions of mesh elements of the patient's dentition (or appliance components or fixture model components) in a manner that can be processed by a computer processor; 2) performing calculation on thousands or millions of mesh elements, e.g., to quantify aspects of the shape and or/structure of a 3D representation of oral care data; 3) for each example in a training dataset of thousands of examples, generating hundreds of iteratively noisier versions of each sample; 4) using those hundreds of thousands of noisy examples to train a denoising ML model; and 5) generating, using the denoising ML model, 3D oral care representations for use in oral care appliance generation (each of which may comprise thousands or millions of mesh elements), and doing so during the course of a short office visit.

In some implementations, a denoising ML model 210 can be trained by providing noisy data from the forward pass 206 to the denoising ML model 210, using the denoising ML model 210 to generate a predicted 3D oral care representation (e.g., a denoised 3D oral care representation), and comparing the predicted 3D oral care representation to a ground truth (or reference) 3D oral care representation. A loss may be computed based on the comparing (e.g., MSE loss, or others described herein), and be used to update the weights of the denoising ML model 210 (e.g., via backpropagation).

1. A method of a generating a data structure for oral care treatment comprising:
receiving, by one or more computer processors, one or more attributes describing an intended output from a trained machine learning model;
generating, by the one or more computer processors, one or more noisy representations of the intended output;
denoising, by the one or more computer processors and using a trained machine learning model, the one or more noisy representations of the intended output;
generating, by the one or more computer processors, one or more denoised representations of the intended output;
automatically defining, by the one or more computer processors and using the one or more generated denoised representations, one or more aspects of one or more digital oral care treatments.

2. The method of claim 1, further comprising: accessing a training dataset;
generating, by the one or more computer processors, a refined training data set by successively modifying one or more representations of the training dataset;
training, by the one or more computer processors, an untrained machine learning model using the refined training dataset; and
and outputting the trained machine learning model based on the training.

3. The method of claim 2, wherein the modifying comprises adding noise to aspects of the one or more representations of the training dataset.

4. The method of claim 2, further comprising encoding, by the one or more computer processors, one or more aspects of one or more representations of the training dataset into a latent form before generating the one or more noisy representations.

5. The method of claim 1, wherein the one or more attributes include at least one of a real value, a categorical value or a natural language text value.

6. The method of claim 4, wherein the one or more attributes include at least one of an oral care metric or an oral care parameter.

7. The method of claim 1, wherein the trained machine learning model includes at least one neural network.

8. The method of claim 7, wherein the at least one neural network includes at least one of a U-Net, a transformer or an autoencoder.

9. The method of claim 1, wherein the method receives one or more 3D representations of the patient's dentition, which includes at least one tooth.

10. The method of claim 9, wherein the one or more denoised representations includes at least one 3D oral care representation.

11. The method of claim 10, wherein the one or more 3D representations of the patient's dentition are encoded into a latent form.

12. The method of claim 10, wherein the one or more denoised representations includes at least one label for a mesh element.

13. The method of claim 12, further comprising segmenting, using the at least one label, one or more 3D representations of oral care data.

14. The method of claim 13, wherein the one or more 3D oral care representations includes at least one representation of a patient's dentition.

15. The method of claim 12, further comprising modifying, using the at least one label, one or more 3D representations of oral care data.

16. The method of claim 15, wherein the one or more 3D oral care representations includes at least one representation of a patient's dentition.

17. The method of claim 10, wherein the one or more denoised representations includes at least one transform to place at least one tooth of a patient's dentition into a setup pose.

18. The method of claim 17, wherein the setup corresponds to a final setup.

19. The method of claim 10, wherein the 3D oral care representation comprises one or more of: a fixture model component, a tooth restoration design, an appliance component, a fixture model component and an archform.

20. The method of claim 10, wherein the one or more denoised representations include one or more coordinate systems.
